(19) United States
(12) Patent Application Publication
(10) Pub. No.: US 2023/0416202 A1
(43) Pub. Date: Dec. 28, 2023

(54) Small Molecule Compounds and Compositions

(71) Applicant: Telo Therapeutics, Inc., San Diego, CA (US)

(72) Inventors: Katherine Widdowson, San Diego, CA (US); Robert Joseph Allen Bell, San Francisco, CA (US); Santosh Kulkarni, Bengaluru (IN); Adrian Hall, Braine l'Alleud (BE); Mark Patrick Healy, Cambridge, MA (US); David George Hubert Livermore, Herts (GB); James William Myatt, Cheshire (US); Mairi Simie, Cumbernauld (GB)

(21) Appl. No.: 18/037,935

(22) PCT Filed: Nov. 19, 2021
(86) PCT No.: PCT/US2021/060000

§ 371 Date: May 19, 2023

Related U.S. Application Data
(60) Provisional application No. 63/278,041, filed on Nov. 10, 2021.
(60) Provisional application No. 63/162,049, filed on Mar. 17, 2021.
(60) Provisional application No. 63/115,650, filed on Nov. 19, 2020.

(51) Int. Cl. C07D 211/34; A61P 35/00; A61K 47/20

(52) U.S. Cl. CPC C07D 211/34; A61P 35/00; A61K 47/20

(57) ABSTRACT

Provided herein are compounds and compositions that inhibit telomerase and/or the TERT gene with a mutant promoter. Further provided are small molecule compounds or compositions having beneficial effects in cancer treatment. Methods of making the compounds and compositions and methods of using the compounds and compositions, such as using the compounds and compositions to treat cancer, are also provided.

### CROSS-REFERENCE TO RELATED APPLICATIONS

This application claims priority to U.S. Provisional Application No. 63/278,041, filed Nov. 10, 2021, entitled SMALL MOLECULE COMPOUNDS AND COMPOSITIONS, U.S. Provisional Application No. 63/162,049, filed Mar. 17, 2021, entitled COMPOUNDS AND COMPOSITIONS FOR INHIBITING TERT EXPRESSION, and U.S. Provisional Application No. 63/115,650, filed Nov. 19, 2020, entitled COMPOUNDS AND COMPOSITIONS FOR INHIBITING TERT EXPRESSION, the contents of each of which are incorporated herein by reference in its entirety.

### FIELD OF DISCLOSURE

The disclosure relates to small molecule compounds and compositions and methods for treating cancer.

### BACKGROUND

Telomerase expression is a hallmark of tumorigenesis. Due to its fundamental nature in driving tumorigenesis, many attempts have been made to inhibit telomerase as a cancer therapeutic strategy, but thus far none have become a standard of care. One promising approach was the oligonucleotide therapy GRN163L from Geron, Inc. By hybridizing and inhibiting the RNA template of telomerase, GRN163L reduced tumor growth in preclinical models of breast cancer, glioblastoma (GBM), pancreatic, and liver cancer. However, this preclinical success has not translated to the clinic, as trials in breast, lung, and pediatric CNS cancers were discontinued. In each case, a high frequency of grade III/IV hematopoietic toxicities were observed. This was thought to result from inhibiting telomerase activity in healthy hematopoietic stem cells. Therefore, there is currently a large unmet need to effectively inhibit telomerase activity selectively in cancer cells.

### SUMMARY OF DISCLOSURE

The present disclosure provides compositions and methods for treating cancer. The present disclosure also provides compounds that inhibit the expression of the TERT gene with a mutant promoter and/or reduce the amount TERT mRNA or TERT proteins in cell with a mutant TERT promoter. Methods of making these compounds and/or compositions are also provided.

In some embodiments, the compound has a structure of Formula (I):

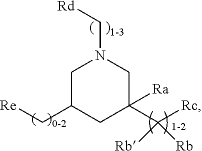

or a pharmaceutically acceptable salt thereof, wherein Ra, each individual Rb and each individual Rb′ can be independently H, halogen (such as F, Cl, Br) or optionally substituted C1-C4 alkyl (e.g., methyl); alternatively, Ra and Rb can be joined to form a 5 or 6 membered ring;
Rc is carboxylic acid or its isostere;
Rd is an optionally substituted aryl or heteroaryl group; and
Re is an optionally substituted aryl or heteroaryl group.

In some embodiments, the compounds of the present disclosure have a general structure of Formula (II):

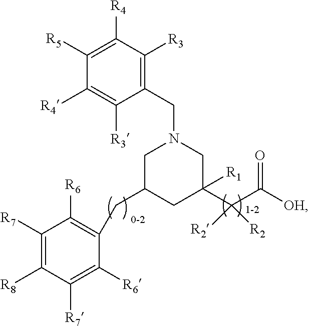

or a pharmaceutically acceptable salt thereof, wherein R1, each individual R2 and each individual R2′ can be independently H, halogen (such as F, Cl and Br) or optionally substituted C1-C4 alkyl (e.g., methyl); alternatively, R1 and R2 can be joined to form a 5 or 6 membered ring; R3, R3′, R4, R4′ or R5 is independently hydrogen, halogen, optionally substituted alkyl, optionally substituted alkenyl, optionally substituted alkynyl, optionally substituted alkoxyl, optionally substituted amine, optionally substituted amide, optionally substituted sulfone, optionally substituted heteroaryl, azide (N3), nitrile (CN), or CF3; or R4 and R5 together with the carbon atoms they are attached to form an optionally substituted aromatic ring, wherein at least 3 of R3, R3′, R4, R4′ and R5 are H; and
R6, R6′, R7, R7′ or R8 is independently hydrogen, halogen, optionally substituted alkyl, optionally substituted alkenyl, optionally substituted alkynyl, optionally substituted alkoxyl, optionally substituted amine, optionally substituted amide, optionally substituted sulfone, optionally substituted heteroaryl, azide (N3), nitrile (CN), or CF3, wherein at least 3 of R6, R6′, R7, R7′ and R8 are H.

In some embodiments, the compounds of the present disclosure have a general structure of Formula (III):

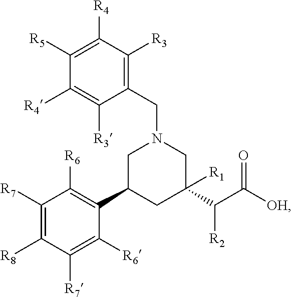

or a pharmaceutically acceptable salt thereof, wherein R1 and R2 can be independently H or optionally substituted C1-C4 alkyl (e.g., methyl); alternatively, R1 and R2 can be joined to form a 5 or 6 membered ring; R3, R3′, R4, R4′ or R5 is independently hydrogen, halogen, optionally substituted alkyl, optionally substituted alkenyl, optionally substituted alkynyl, optionally substituted alkoxyl, optionally substituted amine, optionally substituted amide, optionally substituted sulfone, optionally substituted heteroaryl, azide (N3), nitrile (CN), or CF3, wherein at least 3 of R3, R3′, R4, R4′ and R5 are H; and
R6, R6′, R7, R7′ or R8 is independently hydrogen, halogen, optionally substituted alkyl, optionally substituted alkenyl, optionally substituted alkynyl, optionally substituted alkoxyl, optionally substituted amine, optionally substituted amide, optionally substituted sulfone, optionally substituted heteroaryl, azide (N3), nitrile (CN), or CF3, wherein at least 3 of R6, R6′, R7, R7′ and R8 are H.

In some embodiments, the compound is Compound 100, 101, 102, 103, 104, 105, 106,107, 108, 109, 110, 111, 112, 113, 114,115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, or 149, or a pharmaceutically acceptable salt thereof.

In some embodiments, the present disclosure provides a pharmaceutical composition, comprising any of the compounds and a pharmaceutically acceptable carrier. The present disclosure provides a medicament, comprising any of the compounds and a pharmaceutically acceptable carrier.

In some embodiments, the present disclosure provides a method of inhibiting the expression of the telomerase reverse transcriptase (TERT) gene with a mutant promoter, reducing the amount of TERT mRNAs or TERT proteins in a cell with a mutant TERT promoter with one or more mutations, comprising administering the compounds or pharmaceutical compositions described herein. In some embodiments, the mutation of the TERT promoter is a somatic mutation. In some embodiments, the cell is a cancer cell. In some embodiments, the compounds or pharmaceutical compositions do not inhibit the expression of wild-type TERT genes or reduce the amount of TERT mRNAs or TERT proteins in cells having wild-type TERT genes, wherein the wild-type TERT genes do not have any mutations in the promoters.

In some embodiments, the present disclosure provides a method of treating cancer, reducing tumor volume, reducing tumor growth, and/or increasing survival of a subject comprising administering the compounds or pharmaceutical compositions described herein to the subject.

In some embodiments, the present disclosure provides a use of any of the compounds described herein for the manufacture of a pharmaceutical composition for treating cancer.

In some embodiments, the present disclosure provides the compounds or pharmaceutical compositions described herein for use in treating cancer.

### BRIEF DESCRIPTION OF THE DRAWINGS

The foregoing and other objects, features and advantages will be apparent from the following description of particular embodiments of the disclosure, as illustrated in the accompanying drawings in which like reference characters refer to the same parts throughout the different views. The drawings are not necessarily to scale, emphasis instead being placed upon illustrating the principles of various embodiments of the disclosure.

FIG. 1 shows study design of the in vivo study in Example 3.

FIG. 2 shows % changes in tumor BLI after treatment with Compound 101 BID (50 mg/kg, 100 mg/kg or 200 mg/kg).

FIG. 3 shows % changes in TERT expression after treatment with Compound 101 BID (50 mg/kg, 100 mg/kg or 200 mg/kg).

FIG. 4 shows % changes in tumor BLI after treatment with Compound 101 (50 mg/kg BID, 100 mg/kg BID, or 100 mg/kg QD).

### DETAILED DESCRIPTION

The present disclosure provides compounds and compositions and methods of using the compounds and compositions for inhibiting the expression of the telomerase reverse transcriptase (TERT) gene with a mutant promoter. The TERT gene encodes the catalytic subunit of telomerase and its transcriptional regulation is the rate-limiting step in telomerase activity. Telomerase expression is a hallmark of tumorigenesis and over 90% of human cancers aberrantly express the enzyme. Telomerase functions by elongating telomeres, the ‘TTAGGG’ DNA repeats at the end of chromosomes. The majority of normal tissues have no telomerase activity so that telomeres shorten with each successive round of cell division. Eventually, a critical telomere length is reached, and cells enter replicative senescence or undergo apoptosis. Telomerase reverse transcriptase (TERT) is a catalytic subunit of telomerase which catalyzes the addition of nucleotides in a specific DNA sequence to the ends of a chromosome's telomeres. This addition of repetitive DNA sequences prevents degradation of the chromosomal ends after multiple rounds of replication. Reactivation of TERT expression occurs in many human cancers and TERT reactivation is necessary to overcome replicative senescence (aging) and prevent apoptosis (cell death), both fundamental steps in the initiation of cancer.

The present disclosure also provides compounds and compositions and methods of using the compounds and compositions to treat individual subjects having a disease, disorder and/or condition such as, but not limited to for example, cancer or for reducing tumor volume, reducing tumor growth, and/or increasing survival.

### I. Compounds of the Disclosure

Applicant has used multiple assays to drive therapeutic drug discovery. In general, the compounds of the present disclosure are small molecule compounds having a piperidine core described below. In some embodiments, the molecular weight (MW) of the compound may not be more than 500 g/mol. In some embodiments, the molecular weight (MW) of the compound may not be less than 400 g/mol.

### Generic Structures:

In some embodiments, the compounds of the present disclosure have a general structure of Formula (I):

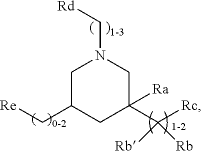

or a pharmaceutically acceptable salt thereof, wherein Ra, each individual Rb and each individual Rb′ can be independently H, halogen (such as F, Cl, Br) or optionally substituted C1-C4 alkyl (e.g., methyl); alternatively, Ra and Rb can be joined to form a 5 or 6 membered ring:
Rc is carboxylic acid or its isostere;
Rd is an optionally substituted aryl or heteroaryl group; and
Re is an optionally substituted aryl or heteroaryl group.

In some embodiments, Rd and/or Re is a phenyl group. In some embodiments, Rd and/or Re is a phenyl group with at least one substituent in the ortho, para or meta position(s). In some embodiments, Rd and/or Re is a phenyl group with 2 substituents, wherein the 2 substituents are in the (3, 5) or (3, 4) positions.

In some embodiments, the substituent(s) for Rd or Re is halogen, optionally substituted alkyl, optionally substituted alkenyl, optionally substituted alkynyl, optionally substituted alkoxyl, optionally substituted amine, optionally substituted amide, optionally substituted sulfone, optionally substituted heteroaryl, azide (N3), nitrile (CN), Cl, F, or CF3.

In some embodiments, the optional substituent includes hydroxyl, methoxy, ethoxy, dimethyl amino, diethyl amino, fluoro, chloro, bromo, CN, CONH2, CON(CH3)2, SO2NH2, SO2NHCH3, or SO2CH3.

In some embodiments, Rb is H, CH3 (Me) or F.

In some embodiments, Rb′ is H, Me or F.

In some embodiments, Rc is —COOH. In some embodiments, Rc is a carboxylic acid isostere such as but not limited to hydroxamic acid, acylcyanamide, sulfonimide, phosphonate, sulfonate, sulfonamide, tetrazole, hydroxyisoxazole, or oxadiazolone. Non-limiting examples of compounds encompassed by Formula (I) include Compounds 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148 and 149.

In some embodiments, the compounds of the present disclosure have a general structure of Formula (II):

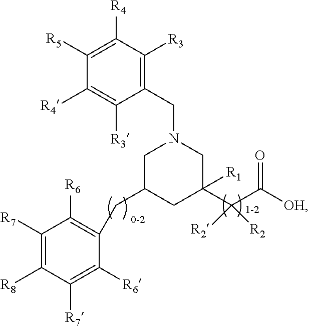

or a pharmaceutically acceptable salt thereof, wherein R1, each individual R2 and each individual R2′ can be independently H, halogen (such as F, Cl and Br) or optionally substituted C1-C4 alkyl (e.g., methyl); alternatively, R1 and R2 can be joined to form a 5 or 6 membered ring; R3, R3′, R4, R4′ or R5 is independently hydrogen, halogen, optionally substituted alkyl, optionally substituted alkenyl, optionally substituted alkynyl, optionally substituted alkoxyl, optionally substituted amine, optionally substituted amide, optionally substituted sulfone, optionally substituted heteroaryl, azide (N3), nitrile (CN), or CF3; or R4 and R5 together with the carbon atoms they are attached to form an optionally substituted aromatic ring, wherein at least 3 of R3, R3′, R4, R4′ and R5 are H; and
R6, R6′, R7, R7′ or R8 is independently hydrogen, halogen, optionally substituted alkyl, optionally substituted alkenyl, optionally substituted alkynyl, optionally substituted alkoxyl, optionally substituted amine, optionally substituted amide, optionally substituted sulfone, optionally substituted heteroaryl, azide (N3), nitrile (CN), or CF3, wherein at least 3 of R6, R6′, R7, R7′ and R8 are H.

In some embodiments, R1 is H. In some embodiments, R1 is —CH3. In some embodiments, R1 is —CH2OH.

In some embodiments, R2 is H. In some embodiments, R2 is —CH3. In some embodiments, R2 is —CH2CH3.

In some embodiments, both R1 and R2 are H.

In some embodiments, R2 is H, Me or F.

In some embodiments, R2′ is H, Me or F.

In some embodiments, 3 of R3, R3′, R4, R4′ or R5 are H; the other two are independently —CF3, —OMe, Cl, —CN, F, SO2Me, N3, CH2N3,

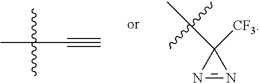

In some embodiments, 4 of R3, R3′, R4, R4′ or R5 are H; the other one is —CF3, —OMe, Cl, —CN, F, SO2Me, N3, CH2N3,

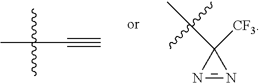

In some embodiments, R3 and R3′ are H; R4, R4′ or R5 is independently H, —CF3, —OMe, —CN, F, SO2Me, N3, CH2N3,

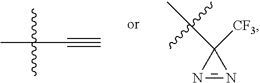

wherein at least one of R4, R4′ or R5 is H.

In some embodiments, 3 of R6, R6′, R7, R7′ or R8 are H; the other two are independently —CF3, —OMe, —CN, F, Cl, N3 or

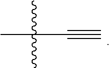 [SMILES: *C#C]

In some embodiments, 4 of R6, R6′, R7, R7′ or R8 are H; the other one is —CF3, —OMe, —CN, F, Cl, N3 or

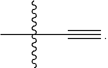 [SMILES: *C#C]

In some embodiments, R6 and R6′ are H; R7, R7′ or R8 is independently H, —CF3, —OMe, —CN, F, Cl, N3 or

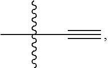 [SMILES: *C#C]

wherein at least one of R7, R7′ or R8 is H.

Non-limiting examples of compounds encompassed by Formula (II) include Compounds 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148 and 149.

In some embodiments, the compounds of the present disclosure have a general structure of Formula (III):

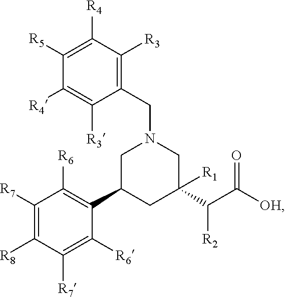

or a pharmaceutically acceptable salt thereof, wherein R1 and R2 can be independently H or optionally substituted C1-C4 alkyl (e.g., methyl); alternatively, R1 and R2 can be joined to form a 5 or 6 membered ring; R3, R3′, R4, R4′ or R5 is independently hydrogen, halogen, optionally substituted alkyl, optionally substituted alkenyl, optionally substituted alkynyl, optionally substituted alkoxyl, optionally substituted amine, optionally substituted amide, optionally substituted sulfone, optionally substituted heteroaryl, azide (N3), nitrile (CN), or CF3, wherein at least 3 of R3, R3′, R4, R4′ and R5 are H; and
R6, R6′, R7, R7′ or R8 is independently hydrogen, halogen, optionally substituted alkyl, optionally substituted alkenyl, optionally substituted alkynyl, optionally substituted alkoxyl, optionally substituted amine, optionally substituted amide, optionally substituted sulfone, optionally substituted heteroaryl, azide (N3), nitrile (CN), or CF3, wherein at least 3 of R6, R6′, R7, R7′ and R8 are H.

In some embodiments, R1 is H. In some embodiments, R1 is —CH3. In some embodiments, R1 is —CH2OH.

In some embodiments, R2 is H. In some embodiments, R2 is —CH3. In some embodiments, R2 is —CH2CH3.

In some embodiments, both R1 and R2 are H.

In some embodiments, 3 of R3, R3′, R4, R4′ or R5 are H; the other two are independently —CF3, —OMe, —CN, —Cl, —F, —SO2Me, —N3, —CH2N3,

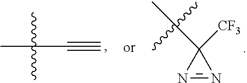

In some embodiments, 4 of R3, R3′, R4, R4′ or R5 are H; the other one is —CF3, —OMe, —CN, —Cl, —F, —SO2Me, —N3, —CH2N3,

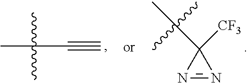

In some embodiments, R3 and R3′ are H; R4, R4′ or R5 is independently H, —CF3, —OMe, —CN, —Cl, —F, —SO2Me, —N3, —CH2N3,

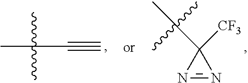

wherein at least one of R4, R4′ or R5 is H.

In some embodiments, 3 of R6, R6′, R7, R7′ or R8 are H; the other two are independently —CF3, —OMe, —CN, —Cl, —F, —SO2Me, —N3, —CH2N3,

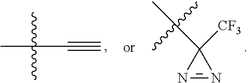

In some embodiments, 4 of R6, R6′, R7, R7′ or R8 are H; the other one is —CF3, —OMe, —CN, —Cl, —F, —SO2Me, —N3, —CH2N3,

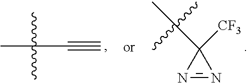

In some embodiments, R6 and R6′ are H; R7, R7′ or R8 is independently H, —CF3, —OMe, —CN, —Cl, —F, —SO2Me, —N3, —CH2N3,

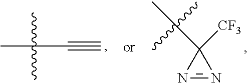

wherein at least one of R7, R7′ or R8 is H.

Non-limiting examples of compounds encompassed by Formula (III) include Compounds 100, 101, 102, 103, 104, 105, 106, 107, 108, 109, 110, 111, 112, 113, 114, 115, 116, 117, 118, 119, 120, 121, 122, 123, 124, 125, 127, 128, 129, 130, 131, 132, 134, 135, 136, 137, 138, 139, 140, 142, 143, 144, 145, 146 and 148.

In some embodiments, compounds of the present disclosure are 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, or 149, or a pharmaceutically acceptable salt thereof.

TABLE 1
Non-Limiting Examples of Compounds of the Disclosure

| Compound No. | Structures |
|---|---|
| 100 (racemic) | 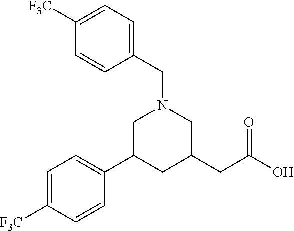 [SMILES: O=C(O)CC1CC(c2ccc(C(F)(F)F)cc2)CN(Cc2ccc(C(F)(F)F)cc2)C1] |
| 101 | 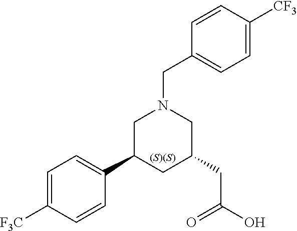 |
| 102 | 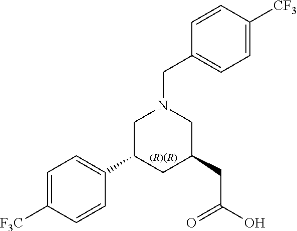 |
| 103 (racemic) | 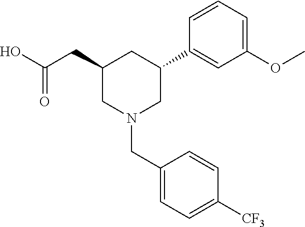 [SMILES: COc1cccc([C@H]2C[C@H](CC(=O)O)CN(Cc3ccc(C(F)(F)F)cc3)C2)c1] |
| 104 (racemic) | 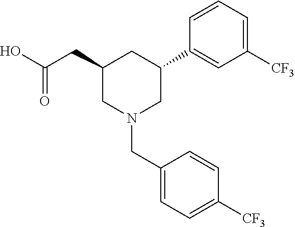 [SMILES: O=C(O)C[C@H]1C[C@H](c2cccc(C(F)(F)F)c2)CN(Cc2ccc(C(F)(F)F)cc2)C1] |
| 105 (racemic) | 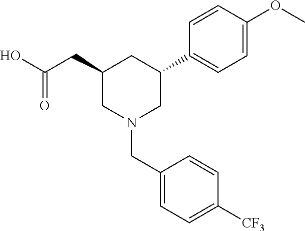 [SMILES: COc1ccc([C@H]2C[C@H](CC(=O)O)CN(Cc3ccc(C(F)(F)F)cc3)C2)cc1] |
| 106 (racemic) | 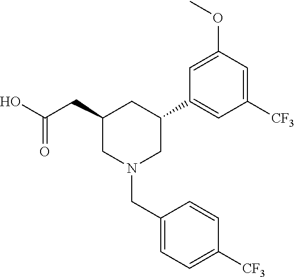 [SMILES: COc1cc([C@H]2C[C@H](CC(=O)O)CN(Cc3ccc(C(F)(F)F)cc3)C2)cc(C(F)(F)F)c1] |
| 107 (racemic) | 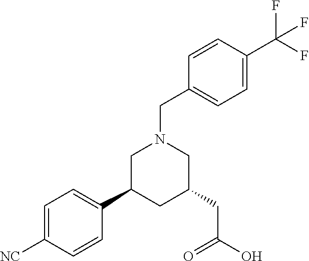 [SMILES: N#Cc1ccc([C@@H]2C[C@@H](CC(=O)O)CN(Cc3ccc(C(F)(F)F)cc3)C2)cc1] |
| 108 | 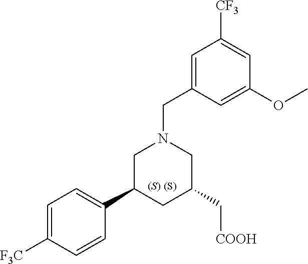 [SMILES: COc1cc(CN2C[C@H](CC(=O)O)C[C@@H](c3ccc(C(F)(F)F)cc3)C2)cc(C(F)(F)F)c1] |
| 109 | 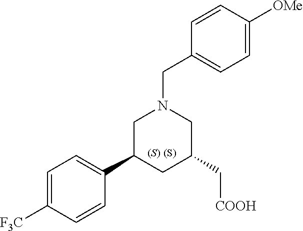 [SMILES: COc1ccc(CN2C[C@H](CC(=O)O)C[C@@H](c3ccc(C(F)(F)F)cc3)C2)cc1] |
| 110 | 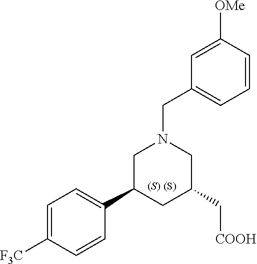 [SMILES: COc1cccc(CN2C[C@H](CC(=O)O)C[C@@H](c3ccc(C(F)(F)F)cc3)C2)c1] |
| 111 | 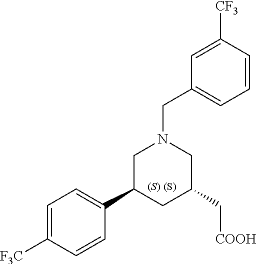 [SMILES: O=C(O)C[C@@H]1C[C@@H](c2ccc(C(F)(F)F)cc2)CN(Cc2cccc(C(F)(F)F)c2)C1] |
| 112 | 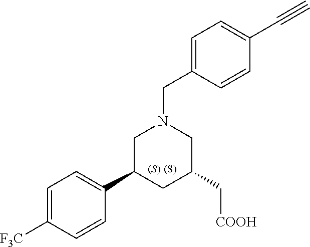 [SMILES: C#Cc1ccc(CN2C[C@H](CC(=O)O)C[C@@H](c3ccc(C(F)(F)F)cc3)C2)cc1] |
| 113 | 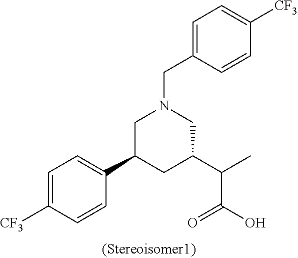 [SMILES: CC(C(=O)O)[C@@H]1C[C@@H](c2ccc(C(F)(F)F)cc2)CN(Cc2ccc(C(F)(F)F)cc2)C1] |
| 114 | 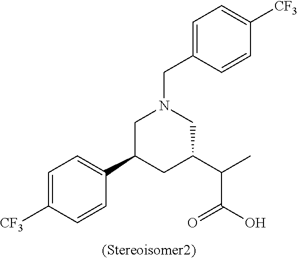 [SMILES: CC(C(=O)O)[C@@H]1C[C@@H](c2ccc(C(F)(F)F)cc2)CN(Cc2ccc(C(F)(F)F)cc2)C1] |
| 115 | 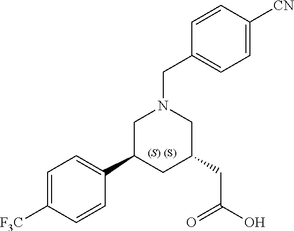 [SMILES: N#Cc1ccc(CN2C[C@H](CC(=O)O)C[C@@H](c3ccc(C(F)(F)F)cc3)C2)cc1] |
| 116 | 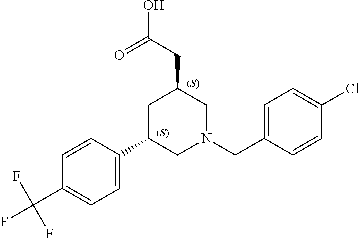 |
| 117 | 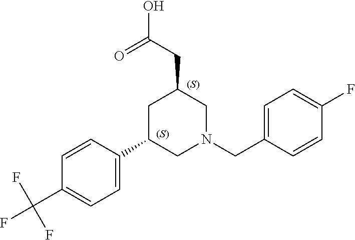 |
| 118 | 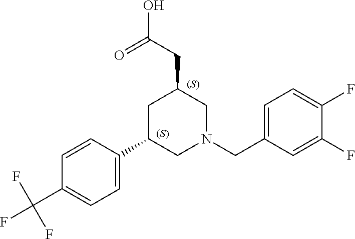 |
| 119 | 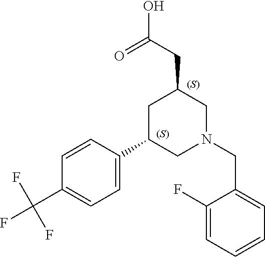 |
| 120 | 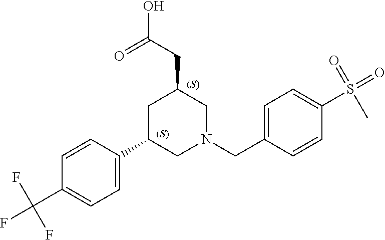 |
| 121 (racemic) | 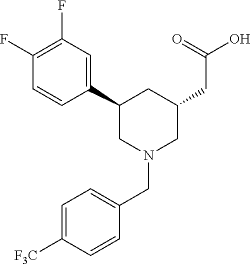 [SMILES: O=C(O)C[C@H]1C[C@H](c2ccc(F)c(F)c2)CN(Cc2ccc(C(F)(F)F)cc2)C1] |
| 122 (racemic) | 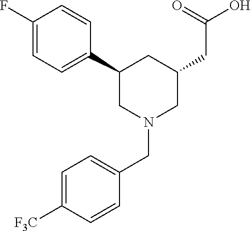 [SMILES: O=C(O)C[C@H]1C[C@H](c2ccc(F)cc2)CN(Cc2ccc(C(F)(F)F)cc2)C1] |
| 123 (racemic) | 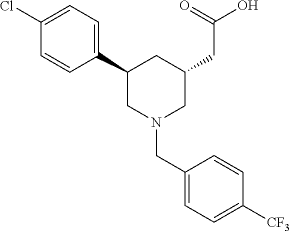 [SMILES: O=C(O)C[C@H]1C[C@H](c2ccc(Cl)cc2)CN(Cc2ccc(C(F)(F)F)cc2)C1] |
| 124 (racemic) | 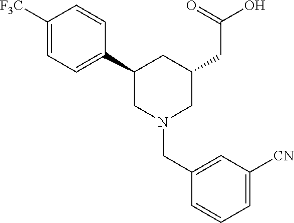 [SMILES: N#Cc1cccc(CN2C[C@@H](CC(=O)O)C[C@H](c3ccc(C(F)(F)F)cc3)C2)c1] |
| 125 (racemic) | 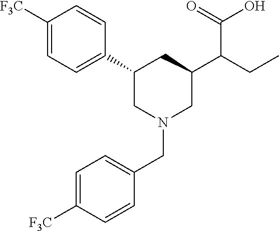 [SMILES: CCC(C(=O)O)[C@@H]1C[C@@H](c2ccc(C(F)(F)F)cc2)CN(Cc2ccc(C(F)(F)F)cc2)C1] |
| 126 (racemic) | 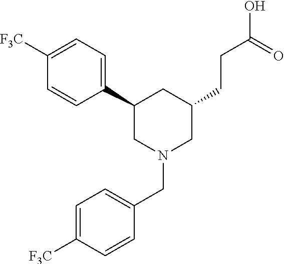 [SMILES: O=C(O)CC[C@H]1C[C@H](c2ccc(C(F)(F)F)cc2)CN(Cc2ccc(C(F)(F)F)cc2)C1] |
| 127 (racemic) | 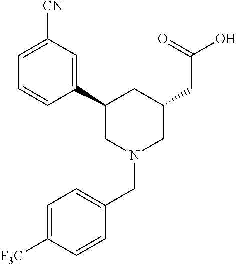 [SMILES: N#Cc1cccc([C@H]2C[C@H](CC(=O)O)CN(Cc3ccc(C(F)(F)F)cc3)C2)c1] |
| 128 (racemic) | 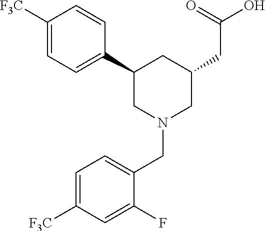 [SMILES: O=C(O)C[C@H]1C[C@H](c2ccc(C(F)(F)F)cc2)CN(Cc2ccc(C(F)(F)F)cc2F)C1] |
| 129 (racemic) | 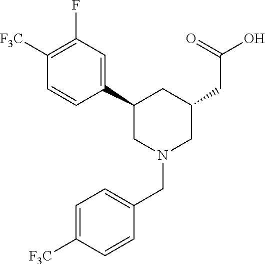 [SMILES: O=C(O)C[C@H]1C[C@H](c2ccc(C(F)(F)F)c(F)c2)CN(Cc2ccc(C(F)(F)F)cc2)C1] |
| 130 (racemic) | 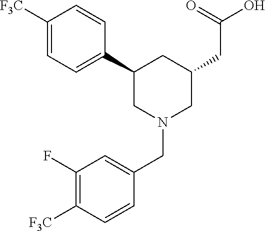 [SMILES: O=C(O)C[C@H]1C[C@H](c2ccc(C(F)(F)F)cc2)CN(Cc2ccc(C(F)(F)F)c(F)c2)C1] |
| 131 (racemic) | 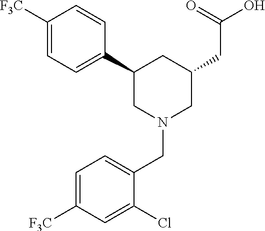 [SMILES: O=C(O)C[C@H]1C[C@H](c2ccc(C(F)(F)F)cc2)CN(Cc2ccc(C(F)(F)F)cc2Cl)C1] |
| 132 (racemic) | 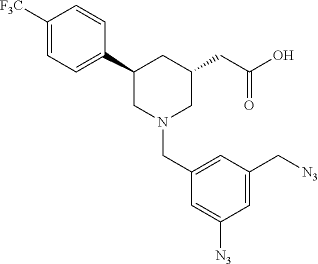 [SMILES: [N-]=[N+]=NCc1cc(CN2C[C@@H](CC(=O)O)C[C@H](c3ccc(C(F)(F)F)cc3)C2)cc(N=[N+]=[N-])c1] |
| 133 (racemic) | 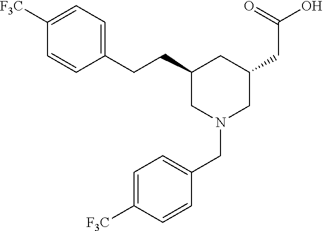 [SMILES: O=C(O)C[C@H]1C[C@H](CCc2ccc(C(F)(F)F)cc2)CN(Cc2ccc(C(F)(F)F)cc2)C1] |
| 134 (racemic) | 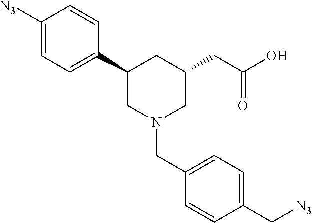 [SMILES: [N-]=[N+]=NCc1ccc(CN2C[C@@H](CC(=O)O)C[C@H](c3ccc(N=[N+]=[N-])cc3)C2)cc1] |
| 135 (racemic) | 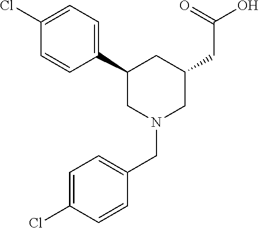 [SMILES: O=C(O)C[C@H]1C[C@H](c2ccc(Cl)cc2)CN(Cc2ccc(Cl)cc2)C1] |
| 136 (racemic) | 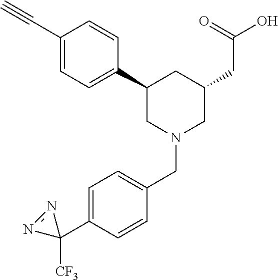 [SMILES: C#Cc1ccc([C@H]2C[C@H](CC(=O)O)CN(Cc3ccc(C4(C(F)(F)F)N=N4)cc3)C2)cc1] |
| 137 (racemic) | 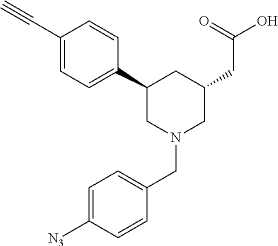 [SMILES: C#Cc1ccc([C@H]2C[C@H](CC(=O)O)CN(Cc3ccc(N=[N+]=[N-])cc3)C2)cc1] |
| 138 (racemic) | 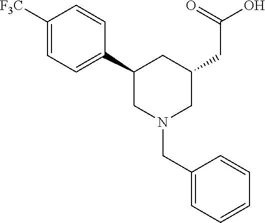 [SMILES: O=C(O)C[C@H]1C[C@H](c2ccc(C(F)(F)F)cc2)CN(Cc2ccccc2)C1] |
| 139 | 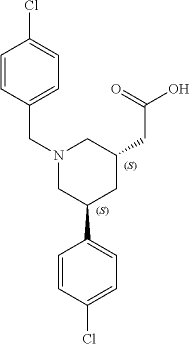 |
| 140 (racemic) | 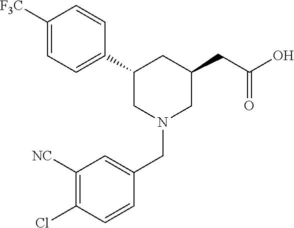 [SMILES: N#Cc1cc(CN2C[C@H](CC(=O)O)C[C@@H](c3ccc(C(F)(F)F)cc3)C2)ccc1Cl] |
| 141 (racemic) | 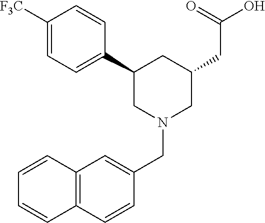 [SMILES: O=C(O)C[C@H]1C[C@H](c2ccc(C(F)(F)F)cc2)CN(Cc2ccc3ccccc3c2)C1] |
| 142 | 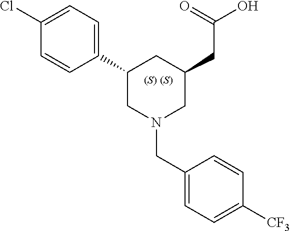 [SMILES: O=C(O)C[C@@H]1C[C@@H](c2ccc(Cl)cc2)CN(Cc2ccc(C(F)(F)F)cc2)C1] |
| 143 (racemic) | 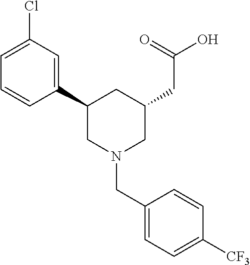 [SMILES: O=C(O)C[C@H]1C[C@H](c2cccc(Cl)c2)CN(Cc2ccc(C(F)(F)F)cc2)C1] |
| 144 | 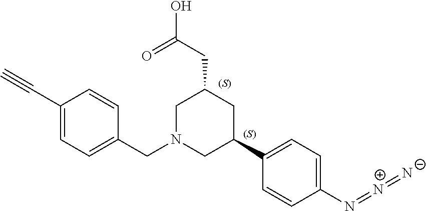 |
| 145 | 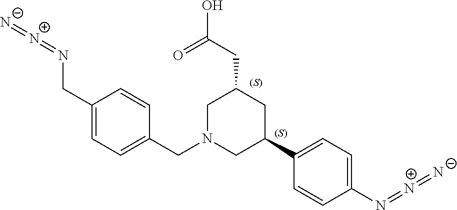 |
| 146 | 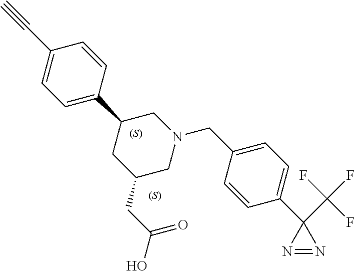 |
| 147 (racemic) | 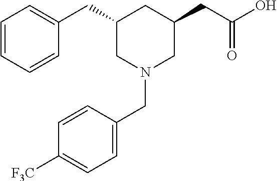 [SMILES: O=C(O)C[C@@H]1C[C@@H](Cc2ccccc2)CN(Cc2ccc(C(F)(F)F)cc2)C1] |
| 148 (racemic) | 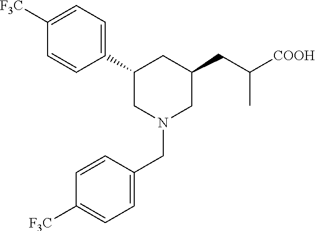 [SMILES: CC(C[C@@H]1C[C@@H](c2ccc(C(F)(F)F)cc2)CN(Cc2ccc(C(F)(F)F)cc2)C1)C(=O)O] |
| 149 (racemic) | 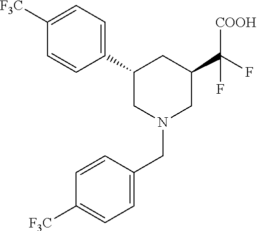 [SMILES: O=C(O)C(F)(F)[C@@H]1C[C@@H](c2ccc(C(F)(F)F)cc2)CN(Cc2ccc(C(F)(F)F)cc2)C1] |

### Formulation

In some embodiments, compositions are administered to humans, human patients or subjects. For the purposes of the present disclosure, the phrase “active ingredient” generally refers to the compounds as described herein. One aspect of the present disclosure provides pharmaceutical compositions comprising at least one pharmaceutically acceptable carrier and a compound of the present disclosure.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

A pharmaceutical composition in accordance with the disclosure may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

The compounds of the present disclosure can be formulated using one or more excipients to: (1) increase stability; (2) permit the sustained or delayed release; (3) alter the biodistribution; (4) alter the release profile of the compounds in vivo. Non-limiting examples of the excipients include any and all solvents, dispersion media, diluents, or other liquid vehicles, dispersion or suspension aids, surface active agents, isotonic agents, thickening or emulsifying agents, and preservatives. Excipients of the present disclosure may also include, without limitation, lipidoids, liposomes, lipid nanoparticles, polymers, lipoplexes, core-shell nanoparticles, peptides, proteins, hyaluronidase, nanoparticle mimics and combinations thereof. Accordingly, the formulations of the disclosure may include one or more excipients, each in an amount that together increases the stability of the compounds.

In some embodiments, pharmaceutical compositions comprising compounds of the present disclosure are provided. The pharmaceutical compositions may be aqueous solutions. In some cases, the aqueous solutions may comprise solutol. The volume percent of solutol may be about 5% to about 15%, such as about 5%, 6%, 7%, 8%, 9%, 10%, 11%, 12%, 13%, 14% or 15%. In some cases, the aqueous solutions may comprise dimethyl sulfoxide (DMSO). The volume percent of DMSO may be between about 1% to about 10%, such as about 1%, 2%, 3%, 4%, 5%, 6%, 7%, 8%, 9% or 10%. In some cases, the aqueous solutions comprise solutol and DMSO. In some cases, the volume ratio of Solutol:DMSO:Water is 10:5:85.

### Excipients

Pharmaceutical formulations may comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, M D, 2006; incorporated herein by reference in its entirety) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this disclosure.

In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical compositions.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and/or combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, and/or combinations thereof.

Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and VEEGUM® [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [TWEEN® 20], polyoxyethylene sorbitan [TWEEN® 60], polyoxyethylene sorbitan monooleate [TWEEN® 80], sorbitan monopalmitate [SPAN040], sorbitan monostearate [SPAN® 60], sorbitan tristearate [SPAN® 65], glyceryl monooleate, sorbitan monooleate [SPAN® 80]), polyoxyethylene esters (e.g. polyoxyethylene monostearate [MYRJ® 45], polyoxyethylene hydrogenated castor oil, polyethoxylated castor oil, polyoxymethylene stearate, and Kolliphor® (SOLUTOL®)), sucrose fatty acid esters, polyethylene glycol fatty acid esters (e.g. CREMOPHOR®), polyoxyethylene ethers, (e.g. polyoxyethylene lauryl ether [BRIJ® 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLUORINC® F 68, POLOXAMER® 188, cetrimonium bromide, cetylpyridinium chloride, benzalkonium chloride, docusate sodium, and/or combinations thereof.

Exemplary binding agents include, but are not limited to, starch (e.g. cornstarch and starch paste); gelatin; sugars (e.g. sucrose, glucose, dextrose, dextrin, molasses, lactose, lactitol, mannitol); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan); alginates; polyethylene oxide; polyethylene glycol; inorganic calcium salts; silicic acid; polymethacrylates; waxes; water; alcohol; and combinations thereof.

Exemplary preservatives may include, but are not limited to, antioxidants, chelating agents, antimicrobial preservatives, antifungal preservatives, alcohol preservatives, acidic preservatives, and/or other preservatives. Exemplary antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Exemplary alcohol preservatives include, but are not limited to, ethanol, polyethylene glycol, phenol, phenolic compounds, bisphenol, chlorobutanol, hydroxybenzoate, and/or phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and/or phytic acid. Other preservatives include, but are not limited to, tocopherol, tocopherol acetate, deteroxime mesylate, cetrimide, butylated hydroxyanisol (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL® 115, GERMABEN® II, NEOLONE™, KATHON™, and/or EUXYL®.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and/or combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and combinations thereof.

Exemplary oils include, but are not limited to, almond, apricot kernel, avocado, babassu, bergamot, black current seed, borage, cade, chamomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and/or combinations thereof.

Excipients such as cocoa butter and suppository waxes, coloring agents, coating agents, sweetening, flavoring, and/or perfuming agents can be present in the composition, according to the judgment of the formulator.

### Delivery and Administration

The present disclosure encompasses the delivery of the compounds and compositions for any therapeutic, prophylactic, pharmaceutical, diagnostic or imaging use by any appropriate route taking into consideration likely advances in the sciences of drug delivery.

The compounds and compositions of the present disclosure may be administered by any route which results in a therapeutically effective outcome. These include, but are not limited to enteral, gastroenteral, epidural, oral, transdermal, epidural (peridural), intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intraarterial (into an artery), intramuscular (into a muscle), intracardiac (into the heart), intraosseous infusion (into the bone marrow), intrathecal (into the spinal canal or into the subarachnoid space to reach the CSF), intraperitoneal, (infusion or injection into the peritoneum), intravesical infusion (e.g., into the bladder using a catheter), intravitreal, (through the eye), intracavernous injection, (into the base of the penis), intravaginal administration, intrauterine, intraparenchymal (into the brain parenchyma), intracerebroventricular (into the cerebrospinal fluid), extra-amniotic administration, transdermal (diffusion through the intact skin for systemic distribution), transmucosal (diffusion through a mucous membrane), insufflation (snorting), sublingual, sublabial, enema, eye drops (onto the conjunctiva), in ear drops, nasal aerosol or inhalation. In specific embodiments, compositions may be administered in a way which allows them to cross the blood-brain barrier, vascular barrier, or other epithelial barrier.

Delivery of the compounds and compositions described herein to a subject over prolonged periods of time, for example, for periods of one week to one year, may be accomplished by a single administration of a controlled release system containing sufficient active ingredient for the desired release period. Various controlled release systems, such as monolithic or reservoir-type microcapsules, depot implants, polymeric hydrogels, osmotic pumps, vesicles, micelles, liposomes, transdermal patches, iontophoretic devices and alternative injectable dosage forms may be utilized for this purpose. Localization at the site to which delivery of the active ingredient is desired is an additional feature of some controlled release devices, which may prove beneficial in the treatment of certain disorders.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant or a similar alcohol.

In some embodiments, the pharmaceutical compositions of the present disclosure may be orally administered in any orally acceptable dosage form including, but not limited to, capsules, tablets, and aqueous suspensions and solutions. In the case of tablets for oral use, carriers that are commonly used include lactose and corn starch. Lubricating agents, such as magnesium stearate, are also typically added. For oral administration in a capsule form, useful diluents include lactose and dried corn starch. When aqueous suspensions are administered orally, the active ingredient is combined with emulsifying and suspending agents. If desired, certain sweetening and/or flavoring and/or coloring agents may be added.

In some embodiments, the pharmaceutical compositions of the present disclosure may be administered by local delivery to the bladder such as, but not limited to, intravesical therapy. Such compositions are prepared according to techniques well-known in the art of pharmaceutical formulation and may be prepared as solutions that can be delivered using, for example, a catheter that is put into the bladder through the urethra.

In some embodiments, the pharmaceutical compositions of the present disclosure may be formulated to be administered to the CNS by routes known in the art such as, but not limited to, direct intraparenchymal administration, intrathecal delivery and intracerebroventricular infusion. In some embodiments, the pharmaceutical compositions are formulated to have the biodistribution of the pharmaceutical composition located in the tumor cells.

In some embodiments, pharmaceutical compositions of the present disclosure may be formulated to improve delivery to tumors.

In some embodiments, pharmaceutical compositions of the present disclosure may be administered via IP (intraperitoneal), SC (subcutaneous) or PO (oral) routes.

### Dosage Forms

A pharmaceutical composition described herein can be formulated into a dosage form described herein, such as a capsule, tablet, aqueous suspension or solution, topical, intranasal, intratracheal, or injectable (e.g., intravenous, intraocular, intravitreal, intramuscular, intracardiac, intraperitoneal, subcutaneous). It will be understood that the total daily usage of the compositions of the present disclosure may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

In some embodiments, compositions in accordance with the present disclosure may be administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, from about 25 mg/kg to about 50 mg/kg, from about 50 mg/kg to about 100 mg/kg, from about 100 mg/kg to about 125 mg/kg, from about 125 mg/kg to about 150 mg/kg, from about 150 mg/to about 175 mg/kg, from about 175 mg/kg to about 200 mg/kg, from about 200 mg/kg to about 250 mg/kg of subject body weight per day, one or more times a day, to obtain the desired therapeutic, diagnostic, prophylactic, or imaging effect. The desired dosage may be delivered three times a day, two times a day, once a day, every other day, every third day, every week, every two weeks, every three weeks, or every four weeks. In some embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used. In some embodiments, the compounds or compositions of the present disclosure are administered by continuous infusion.

As used herein, a “split dose” is the division of single unit dose or total daily dose into two or more doses, e.g, two or more administrations of the single unit dose. As used herein, a “single unit dose” is a dose of any therapeutic administered in one dose/at one time/single route/single point of contact, i.e., single administration event. As used herein, a “total daily dose” is an amount given or prescribed in 24 hr period. It may be administered as a single unit dose.

The administration of the compounds or compositions of the present disclosure can be used as a chronic or acute therapy. The amount of drug that may be combined with the carrier to produce a single dosage form will vary depending upon the host treated and the particular mode of administration. A typical preparation will contain from about 5% to about 95% active compound (w/w). Preferably, such preparations contain from about 20% to about 80%, 30% to about 70%, 40% to about 60%, or about 50% active compound. In other embodiments, the preparations used in the present disclosure will be about 5-10%, 10-20%, 20-30%, 30-40%, 40-50%, 50-60%, 60-70%, 70-80%, 80-90%, 90-99%, or greater than 99% of the active ingredient.

Upon improvement of a patient's condition, a maintenance dose of a compound, composition or combination of the present disclosure may be administered, if necessary. Subsequently, the dosage or frequency of administration, or both, may be reduced, as a function of the symptoms, to a level at which the improved condition is retained when the symptoms have been alleviated to the desired level, treatment should cease. Patients may, however, require intermittent treatment on a long-term basis upon any recurrence of disease symptoms.

As the skilled artisan will appreciate, lower or higher doses than those recited above may be required. Specific dosage and treatment regimens for any particular patient will depend upon a variety of factors, including the activity of the specific compound employed, the age, body weight, general health status, gender, diet, time of administration, rate of excretion, drug combination, the severity and course of an infection, the patient's disposition to the infection and the judgment of the treating physician.

### III. Methods of Use

One aspect of the present disclosure provides methods of using compounds and compositions of the present disclosure. In some embodiments is provided a method of regulating the expression of the TERT gene having a mutant promoter (TERTp) in vitro and/or in vivo comprising administering the compounds and compositions of the present disclosure. TERTp mutations can be detected by any known method in the art, such as PCR and sanger sequencing of the TERT promoter region. They can also be detected by ddPCR and high-throughput sequencing technologies. In some embodiments, the expression of the TERT gene having a mutant promoter is reduced by at least 20%, 30%, 40%, or at least 45%, 50%, 55%, 60%, 65%, 70%, 75%, or at least 80% in the presence of the compounds and compositions of the present disclosure compared to the expression in the absence of the compounds of the present disclosure.

In some embodiments, a method of reducing the amount of TERT mRNAs or TERT proteins in a cell is provided, wherein the cells have mutant TERT promoters, comprising administering the compounds and compositions of the present disclosure. In some embodiments, the amount of TERT mRNAs or TERT proteins is reduced by at least 20%, 30%, 40%, or at least 45%, 50%, 55%, 60%, 65%, 70%, 75%, or at least 80% in the presence of the compounds and compositions of the present disclosure compared to the amount in the absence of the compounds of the present disclosure.

In some embodiments, the compounds or pharmaceutical compositions do not inhibit the expression of wild-type TERT genes or reduce the amount of TERT mRNA or TERT proteins in cells having wild-type TERT genes. The wild-type TERT genes do not have any mutations in the promoters.

Some embodiments provide methods of use of the compounds and compositions described herein to prevent or treat diseases or disorders such as but not limited to cancer or reducing tumor volume, reducing tumor growth, and/or increasing survival. Thus, in some embodiments, the methods provided herein include administering the compounds and compositions described herein to subjects having a cancer. Accordingly, the present disclosure provides methods for treating individual subjects suffering from cancer. In some embodiments, the cancer cells have TERT genes with promoter (TERTp) mutations. In some embodiments, the cancer cells have wild type TERT promoters and do not have promoter mutations.

In some embodiments, the methods of use can be assessed using any endpoint indicating a benefit to the subject, including, without limitation, (1) inhibition, to some extent, of disease progression, including stabilization, slowing down and complete arrest; (2) reduction in the number of disease episodes and/or symptoms; (3) inhibition (i.e., reduction, slowing down or complete stopping) of a disease cell infiltration into adjacent peripheral organs and/or tissues; (4) inhibition (i.e. reduction, slowing down or complete stopping) of disease spread; (5) decrease of an autoimmune condition; (6) favorable change in the expression of a biomarker associated with the disorder; (7) relief, to some extent, of one or more symptoms associated with a disorder; (8) increase in the length of disease-free presentation following treatment; or (9) decreased mortality at a given point of time following treatment

### Cancer

Various cancers may be treated with the compounds and compositions described herein. As used herein, the term “cancer” refers to any of various malignant neoplasms characterized by the proliferation of anaplastic cells that tend to invade surrounding tissue and metastasize to new body sites and also refers to the pathological condition characterized by such malignant neoplastic growths. Cancers may be tumors or hematological malignancies, and include but are not limited to, all types of lymphomas/leukemias, carcinomas and sarcomas, such as those cancers or tumors found in the anus, bladder, bile duct, bone, brain, breast, cervix, colon/rectum, endometrium, esophagus, eye, gallbladder, head and neck, liver, kidney, larynx, lung, mediastinum (chest), mouth, ovaries, pancreas, penis, prostate, skin, small intestine, stomach, spinal marrow, tailbone, testicles, thyroid and uterus.

In some embodiments, the types of carcinomas which may be treated with the compounds and compositions of the present disclosure include, but are not limited to, papilloma/carcinoma, choriocarcinoma, endodermal sinus tumor, teratoma, adenoma/adenocarcinoma, melanoma, atypical fibroxanthoma, fibroma, lipoma, leiomyoma, rhabdomyoma, mesothelioma, angioma, osteoma, chondroma, glioma, lymphoma/leukemia, squamous cell carcinoma, small cell carcinoma, large cell undifferentiated carcinomas, basal cell carcinoma, sinonasal undifferentiated carcinoma and urothelial carcinoma.

In some embodiments, the types of carcinomas which may be treated with the compounds and compositions of the present disclosure include, but are not limited to, soft tissue sarcoma such as alveolar soft part sarcoma, angiosarcoma, dermatofibrosarcoma, desmoid tumor, desmoplastic small round cell tumor, extraskeletal chondrosarcoma, extraskeletal osteosarcoma, fibrosarcoma, hemangiopericytoma, hemangiosarcoma, Kaposi's sarcoma, leiomyosarcoma, liposarcoma, lymphangiosarcoma, lymphosarcoma, malignant fibrous histiocytoma, neurofibrosarcoma, rhabdomyosarcoma, synovial sarcoma, and Askin's tumor, Ewing's sarcoma (primitive neuroectodermal tumor), malignant hemangioendothelioma, malignant schwannoma, osteosarcoma, and chondrosarcoma.

As a non-limiting example, the carcinoma which may be treated by the compounds and compositions of the present disclosure may be Acral melanoma, Acute granulocytic leukemia, Acute lymphocytic leukemia, Acute myelogenous leukemia, Adenocarcinoma, Adenosarcoma, Adrenal cancer, Adrenocortical carcinoma, Anal cancer, Anaplastic astrocytoma, Anaplastic ependymoma, Anaplastic oligodendroglioma, Anaplastic thyroid carcinoma, Angiosarcoma, Appendix cancer, Astrocytoma, Basal cell carcinoma, B-Cell lymphoma, Bile duct cancer, Bladder cancer, Bone cancer, Bowel cancer, Benign thyroid cancer, Brain cancer, Brain stem glioma, Brain tumor, Breast cancer, Carcinoid tumors, Cervical cancer, Cholangiocarcinoma, Chondrosarcoma, Chronic lymphocytic leukemia, Chronic myelogenous leukemia, Clear cell carcinoma, Colon cancer, Colorectal cancer, Craniopharyngioma, Conjuctival melanoma, Cutaneous lymphoma, Cutaneous melanoma, Diffuse astrocytoma, Ductal carcinoma in situ, Endometrial cancer, Ependymoma, Epithelioid sarcoma, Esophageal cancer, Esophageal squamous cell carcinoma, Ewing sarcoma, Extrahepatic bile duct cancer, Eye cancer, Fallopian tube cancer, Fibrosarcoma, Gallbladder cancer, Ganglioglioma, Gastric cancer, Gastrointestinal cancer, Gastrointestinal carcinoid cancer, Gastrointestinal stromal tumors, General, Germ cell tumor, Glioblastoma multiforme, Glioma, Gliosarcoma, Hairy cell leukemia, Head and neck cancer, Head and neck squamous cell carcinoma, Hemangioendothelioma, Hepatocellular carcinoma, Hodgkin lymphoma, Hodgkin's disease, Hodgkin's lymphoma, Hypopharyngeal cancer, Infiltrating ductal carcinoma, Infiltrating lobular carcinoma, Inflammatory breast cancer, Intestinal Cancer, Intrahepatic bile duct cancer, invasive/infiltrating breast cancer, Islet cell cancer, Jaw cancer, Kaposi sarcoma, Kidney cancer, Laryngeal cancer, Leiomyosarcoma, Leptomeningeal metastases, Leukemia, Lip cancer, Liposarcoma, Liver cancer, Lobular carcinoma in situ, Low-grade astrocytoma, Lung adenocarcinoma, Lung cancer, Lymph node cancer, Lymphoma, Male breast cancer, Malignant peripheral nerve sheath tumor, Medullary carcinoma, Medulloblastoma, Melanoma, Meningioma, Merkel cell carcinoma, Mesenchymal chondrosarcoma, Mesothelioma, Metastatic breast cancer, Metastatic melanoma, Metastatic squamous neck cancer, Mixed gliomas, Myxoid liposarcoma, Mouth cancer, Mucinous carcinoma, Mucosal melanoma, Multiple myeloma, Nasal cavity cancer, Nasopharyngeal cancer, Neck cancer, Neuroblastoma, Neurocytoma, Neuroendocrine tumors, Non-Hodgkin lymphoma, Non-Hodgkin's lymphoma, Non-small cell lung cancer, Oat cell cancer, Ocular cancer, Ocular melanoma, Oligoastrocytoma, Oligodendroglioma, Oral cancer, Oral cavity cancer, Oropharyngeal cancer, Osteogenic sarcoma, Osteosarcoma, Ovarian cancer, Ovarian epithelial cancer, Ovarian germ cell tumor, Ovarian primary peritoneal carcinoma, Ovarian sex cord stromal tumor, Paget's disease, Pancreatic cancer, Papillary carcinoma, Paranasal sinus cancer, Parathyroid cancer, Pelvic cancer, Penile cancer, Peripheral nerve cancer, Peritoneal cancer, Pharyngeal cancer, Phaeochromocytoma, Pilocytic astrocytoma, Pineal region tumor, Pineoblastoma, Pituitary gland cancer, Primary central nervous system lymphoma, Pleomorphic dermal sarcoma, Pleomorphic xanthoastrocytoma, Poorly differentiated thyroid carcinoma, Prostate cancer, Rectal cancer, Renal cell cancer, Renal pelvis cancer, Rhabdomyosarcoma, Salivary gland cancer, Sarcoma, Sarcoma, bone, Sarcoma, soft tissue, Sarcoma, uterine, Serous carcinoma, Sinus cancer, Sino nasal malignant melanoma Skin cancer, Small cell lung cancer, Small intestine cancer, Soft tissue sarcoma, Solitary Fibrous tumor Spinal cancer, Spinal column cancer, Spinal cord cancer, Spinal tumor, Spitzoid neoplasm, Squamous cell carcinoma, Squamous cell carcinoma of the cervix, Stomach cancer, Synovial sarcoma, T-cell lymphoma, Tall cell papillary thyroid carcinoma, Testicular cancer, Testicular germ cell tumor, Throat cancer, Thymoma/thymic carcinoma, Thyroid cancer, Thyroid carcinoma, Tongue cancer, Tonsil cancer, Transitional cell cancer, Transitional cell cancer, Transitional cell cancer, Triple-negative breast cancer, Tubal cancer, Tubular carcinoma, Ureteral cancer, Ureteral cancer, Urethral cancer, Uterine adenocarcinoma, Uterine cancer, Uterine sarcoma, Vaginal cancer, and Vulvar cancer.

In some embodiments, compounds and compositions of the present disclosure may be used to treat central nervous system (CNS) tumors, such as but not limited to brain tumor, spinal cord tumor, glioblastoma, meningioma, medulloblastomas, craniopharyngioma, astrocytic tumors, oligodendroglial tumors, mixed gliomas, ependymal tumors, pineal parenchymal tumors, meningeal tumors, or germ cell tumors.

In some embodiments, compounds and compositions of the present disclosure may be used to treat hepatocellular carcinoma (HCC).

### Combination Therapies

In some embodiments, the present invention provides a method of treating a disease or disorder described herein, comprising administering a compound of the present disclosure in combination with one or more additional active agents or therapies. Suitable pharmaceutical agents or therapies that may be used in combination with the compounds of the present disclosure include anti-cancer agents, chemotherapy, and/or immuno-oncology therapy.

The compounds of the present disclosure and the additional active agent(s) may be administered simultaneously, sequentially, or at any order. The compounds of the present disclosure and the additional active agent(s) may be administered at different dosages, with different dosing frequencies, or via different routes, whichever is suitable.

### IV. Kits and Devices

### Kits

The disclosure provides a variety of kits for conveniently and/or effectively carrying out methods of the present disclosure. Typically, kits will comprise sufficient amounts and/or numbers of components to allow a user to perform multiple treatments of a subject(s) and/or to perform multiple experiments.

The kit may further comprise packaging and instructions and/or a delivery agent to form a formulation, e.g., for administration to a subject in need of treatment using the compositions described herein. The delivery agent may comprise a saline, a buffered solution, a lipidoid, a dendrimer or any suitable delivery agent.

In one non-limiting example, the buffer solution may include sodium chloride, calcium chloride, phosphate and/or EDTA. In another non-limiting example, the buffer solution may include, but is not limited to, saline, saline with 2 mM calcium, 5% sucrose, 5% sucrose with 2 mM calcium, 5% Mannitol, 5% Mannitol with 2 mM calcium, Ringer's lactate, sodium chloride, sodium chloride with 2 mM calcium and mannose (See U.S. Pub. No. 20120258046; herein incorporated by reference in its entirety). In yet another non-limiting example, the buffer solutions may be precipitated, or it may be lyophilized. The amount of each component may be varied to enable consistent, reproducible higher concentration saline or simple buffer formulations. The components may also be varied in order to increase the stability of small molecule compositions in the buffer solution over a period of time and/or under a variety of conditions.

### Devices

The present disclosure provides for devices which may incorporate small molecule-based compositions of the present disclosure. These devices can contain a stable formulation available to be immediately delivered to a subject in need thereof, such as a human patient.

Non-limiting examples of the devices include a pump, a catheter, a needle, a transdermal patch, a pressurized olfactory delivery device, electroporation devices, iontophoresis devices, multi-layered microfluidic devices. The devices may be employed to deliver small molecule-based compositions of the present disclosure according to single, multi- or split-dosing regiments. The devices may be employed to deliver small molecule-based compositions of the present disclosure across biological tissue, intradermal, subcutaneously, or intramuscularly.

### V. Definitions

For convenience, the meaning of certain terms and phrases used in the specification, examples, and appended claims, are provided below. If there is an apparent discrepancy between the usage of a term in other parts of this specification and its definition provided in this section, the definition in this section shall prevail.

The abbreviations used herein have their conventional meaning within the scientific arts. The chemical elements are identified in accordance with the Periodic Table of the Elements, CAS version, Handbook of Chemistry and Physics, 75th Ed. Additionally, general principles of organic chemistry are described in M. Loudon, Organic Chemistry, 5th Ed., Roberts and Company, Greenwood Village, Colo.: 2009; and M. B. Smith, March's Advanced Organic Chemistry: Reactions, Mechanisms and Structure, 7th Ed., John Wiley & Sons, Hoboken: 2013, the entire contents of which are hereby incorporated by reference.

As used herein, the term “about” means+/−10% of the recited value.

The term “compound”, as used herein, is meant to include all stereoisomers, geometric isomers, tautomers, and isotopes of the structures depicted.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

Compounds of the present disclosure may also include tautomeric forms. Tautomeric forms result from the swapping of a single bond with an adjacent double bond and the concomitant migration of a proton. Tautomeric forms include prototropic tautomers which are isomeric protonation states having the same empirical formula and total charge. Examples prototropic tautomers include ketone—enol pairs, amide—imidic acid pairs, lactam—lactim pairs, amide—imidic acid pairs, enamine—imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds of the present disclosure also include all of the isotopes of the atoms occurring in the intermediate or final compounds. “Isotopes” refers to atoms having the same atomic number but different mass numbers resulting from a different number of neutrons in the nuclei. For example, isotopes of hydrogen include tritium and deuterium.

The compounds and salts of the present disclosure can be prepared in combination with solvent or water molecules to form solvates and hydrates by routine methods.

Where substituent groups are specified by their conventional chemical formulae, written from left to right, they equally encompass the chemically identical substituents which would result from writing the structure from right to left, e.g., —CH₂O— is intended to also recite —OCH₂—; —NHS(O)₂— is also intended to represent —S(O)₂HN—; etc.

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl.” Alkyl groups, which are limited to hydrocarbon groups are termed “homoalkyl”.

The term “alkylene” by itself or as part of another substituent means a divalent radical derived from an alkane, as exemplified, but not limited, by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The terms “alkoxy,” (or “alkoxyl”) “alkylamino” and “alkylthio” (or thioalkoxy) are used in their conventional sense and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, B and S, and wherein the nitrogen and sulfur atoms may optionally be oxidized and the nitrogen heteroatom may optionally be quaternized. The heteroatom(s) O, N, S and B may be placed at any interior position of the heteroalkyl group or at the position at which the alkyl group is attached to the remainder of the molecule. Examples include, but are not limited to, —CH₂—CH₂—O-CH₃, —CH₂—CH₂—NH—CH₃, —CH₂—CH₂—N(CH₃)—CH₃, —CH₂—S—CH₂—CH₃, —CH₂—CH₂, —S(O)₂—CH₃, —CH₂—CH₂—S(O)₂—CH₃, —CH═CH—O—CH₃, —B(OCH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—B(OH)₂. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—.

In general, an “acyl substituent” is also selected from the group set forth above. As used herein, the term “acyl substituent” refers to groups attached to, and fulfilling the valence of a carbonyl carbon that is either directly or indirectly attached to the polycyclic nucleus of the compounds of the present invention.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, hydrocarbon substituent which can be a single ring or multiple rings (preferably from 1 to 3 rings) which are fused together or linked covalently. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

The terms “carbocycle” and “heterocycle” refers to non-aromatic (such as “cycloalkyl” and “heterocycloalkyl” as defined herein) or aromatic (such as “aryl” and “heteroaryl” as defined herein) rings. The “carbocycle” and “heterocycle” groups may be saturated or non-saturated.

Each of the above terms (e.g., “alkyl,” “heteroalkyl,” “aryl,” “heteroaryl,” “carbocycle,” and “heterocycle”) include both substituted and unsubstituted forms of the indicated radical. Preferred substituents for each type of radical are provided below.

Substituents for the alkyl, and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generally referred to as “alkyl substituents” and “heteroalkyl substituents,” respectively, and they can be one or more of a variety of groups selected from, but not limited to: —OR′, ═O, NR′, ═N—OR′, —NR′R″, —SR′, -halogen, —BO′R″OR′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, the aryl substituents and heteroaryl substituents are generally referred to as “aryl substituents” and “heteroaryl substituents,” respectively and are varied and selected from, for example: halogen, —OR′, ═O, ═NR′, ═N—OR′, —NR′R″, —SR′, -halogen, —BO′R″OR′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, (C₁-C₅)alkyl and heteroalkyl, unsubstituted aryl and heteroaryl, (unsubstituted aryl)-(C₁-C₄)alkyl, and (unsubstituted aryl)oxy-(C₁-C₄)alkyl. When a compound of the invention includes more than one R group, for example, each of the R groups is independently selected as are each R′, R″, R′″ and R″″ groups when more than one of these groups is present.

Two of the aryl substituents on adjacent atoms of the aryl or heteroaryl ring may optionally be replaced with a substituent of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula -A-(CH₂)ᵣ—R″—, wherein A and R″ are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X—CR″R′)_{d}—, where s and d are independently integers of from 0 to 3, and X is —O—, —NR′—, —S—, —S(O)—, —S(O)₂—, or —S(O)₂NR′—. The substituents R, R′, R″ and R′″ are preferably independently selected from hydrogen or substituted or unsubstituted (C₁-C₆)alkyl.

The term “alkyl amide” refers to carboxylic acid amides that are functionalized on the amide nitrogen by one or more alkyl groups as defined herein.

The term “alkyl amine” refers to amines in which the nitrogen atom is functionalized with one or more alkyl groups as defined herein.

As used herein, the term “heteroatom” includes oxygen (O), nitrogen (N), sulfur (S) and silicon (Si).

The symbol “R” is a general abbreviation that represents a substituent group that is selected from substituted or unsubstituted alkyl, substituted or unsubstituted heteroalkyl, substituted or unsubstituted aryl, substituted or unsubstituted heteroaryl, and substituted or unsubstituted heterocyclyl groups.

The term “pharmaceutically acceptable salts” includes salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The neutral forms of the compounds are preferably regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present disclosure provides compounds, which are in a prodrug form. Prodrugs of the compounds described herein are those compounds that readily undergo chemical changes under physiological conditions to provide the compounds of the present invention. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

The terms “subject” or “patient”, as used herein, refer to any organism to which the particles may be administered, e.g., for experimental, therapeutic, diagnostic, and/or prophylactic purposes. Typical subjects include animals (e.g., mammals such as mice, rats, rabbits, guinea pigs, cattle, pigs, sheep, horses, dogs, cats, hamsters, lamas, non-human primates, and humans).

The terms “treating” or “preventing”, as used herein, can include preventing a disease, disorder or condition from occurring in an animal that may be predisposed to the disease, disorder and/or condition but has not yet been diagnosed as having the disease, disorder or condition; inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain.

The terms “managing” or “maintaining”, as used herein, can refer to reducing the symptom(s) of a disease, reducing the severity of symptom(s) of the disease, or preventing the symptom(s) of the disease from getting worse.

The term “therapeutic effect” is art-recognized and refers to a local or systemic effect in animals, particularly mammals, and more particularly humans caused by a pharmacologically active substance. The term thus means any substance intended for use in the diagnosis, cure, mitigation, treatment or prevention of disease, disorder or condition in the enhancement of desirable physical or mental development and conditions in an animal, e.g., a human.

The term “modulation” is art-recognized and refers to up regulation (i.e., activation or stimulation), down regulation (i.e., inhibition or suppression) of a response, or the two in combination or apart. The modulation is generally compared to a baseline or reference that can be internal or external to the treated entity.

“Parenteral administration”, as used herein, means administration by any method other than through the digestive tract (enteral) or non-invasive topical routes. For example, parenteral administration may include administration to a patient intravenously, intradermally, intraperitoneally, intrapleurally, intratracheally, intraossiously, intracerebrally, intrathecally, intramuscularly, subcutaneously, subjunctivally, by injection, and by infusion.

“Topical administration”, as used herein, means the non-invasive administration to the skin, orifices, or mucosa. Topical administration can be delivered locally, i.e., the therapeutic can provide a local effect in the region of delivery without systemic exposure or with minimal systemic exposure. Some topical formulations can provide a systemic effect, e.g., via adsorption into the blood stream of the individual. Topical administration can include, but is not limited to, cutaneous and transdermal administration, buccal administration, intranasal administration, intravaginal administration, intravesical administration, ophthalmic administration, and rectal administration.

“Enteral administration”, as used herein, means administration via absorption through the gastrointestinal tract. Enteral administration can include oral and sublingual administration, gastric administration, or rectal administration.

“Pulmonary administration”, as used herein, means administration into the lungs by inhalation or endotracheal administration. As used herein, the term “inhalation” refers to intake of air to the alveoli. The intake of air can occur through the mouth or nose.

The terms “sufficient” and “effective”, as used interchangeably herein, refer to an amount (e.g., mass, volume, dosage, concentration, and/or time period) needed to achieve one or more desired result(s). A “therapeutically effective amount” is at least the minimum concentration required to affect a measurable improvement or prevention of at least one symptom or a particular condition or disorder, to affect a measurable enhancement of life expectancy, or to generally improve patient quality of life. The therapeutically effective amount is thus dependent upon the specific biologically active molecule and the specific condition or disorder to be treated. Therapeutically effective amounts of many active agents, such as antibodies, are known in the art. The therapeutically effective amounts of compounds and compositions described herein, e.g., for treating specific disorders may be determined by techniques that are well within the craft of a skilled artisan, such as a physician.

The terms “bioactive agent” and “active agent”, as used interchangeably herein, include, without limitation, physiologically or pharmacologically active substances that act locally or systemically in the body. A bioactive agent is a substance used for the treatment (e.g., therapeutic agent), prevention (e.g., prophylactic agent), diagnosis (e.g., diagnostic agent), cure or mitigation of disease or illness, a substance which affects the structure or function of the body, or pro-drugs, which become biologically active or more active after they have been placed in a predetermined physiological environment.

The term “pharmaceutically acceptable”, as used herein, refers to compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio, in accordance with the guidelines of agencies such as the U.S. Food and Drug Administration. A “pharmaceutically acceptable carrier”, as used herein, refers to all components of a pharmaceutical formulation that facilitate the delivery of the composition in vivo. Pharmaceutically acceptable carriers include, but are not limited to, diluents, preservatives, binders, lubricants, disintegrators, swelling agents, fillers, stabilizers, and combinations thereof.

The term “pharmaceutically acceptable salt(s)” refers to salts of acidic or basic groups that may be present in compounds used in the present compositions. Compounds included in the present compositions that are basic in nature are capable of forming a variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to sulfate, citrate, malate, acetate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that include an amino moiety may form pharmaceutically acceptable salts with various amino acids, in addition to the acids mentioned above. Compounds included in the present compositions, that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts.

If the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare non-toxic pharmaceutically acceptable addition salts.

A pharmaceutically acceptable salt can be derived from an acid selected from 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid, aspartic acid, benzenesulfonic acid, benzoic acid, camphoric acid, camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, glucoheptonic acid, gluconic acid, glucuronic acid, glutamic acid, glutaric acid, glycerophosphoric acid, glycolic acid, hippuric acid, hydrobromic acid, hydrochloric acid, isethionic, isobutyric acid, lactic acid, lactobionic acid, lauric acid, maleic acid, malic acid, malonic acid, mandelic acid, methanesulfonic acid, mucic, naphthalene-1,5-disulfonic acid, naphthalene-2-sulfonic acid, nicotinic acid, nitric acid, oleic acid, oxalic acid, palmitic acid, pamoic acid, pantothenic, phosphoric acid, proprionic acid, pyroglutamic acid, salicylic acid, sebacic acid, stearic acid, succinic acid, sulfuric acid, tartaric acid, thiocyanic acid, toluenesulfonic acid, trifluoroacetic, and undecylenic acid.

The term “protective group”, as used herein, refers to a functional group that can be added to and/or substituted for another desired functional group to protect the desired functional group from certain reaction conditions and selectively removed and/or replaced to deprotect or expose the desired functional group. Protective groups are known to the skilled artisan. Suitable protective groups may include those described in Greene and Wuts, Protective Groups in Organic Synthesis, (1991). Acid sensitive protective groups include dimethoxytrityl (DMT), tert-butylcarbamate (tBoc) and trifluoroacetyl (tFA). Base sensitive protective groups include 9-fluorenylmethoxycarbonyl (Fmoc), isobutyri (iBu), benzoyl (Bz) and phenoxyacetyl (pac). Other protective groups include acetamidomethyl, acetyl, tert-amyloxycarbonyl, benzyl, benzyloxycarbonyl, 2-(4-biphenylyl)-2-propyloxycarbonyl, 2-bromobenzyloxycarbonyl, tert-butyl 7 tert-butyloxycarbonyl, l-carbobenzoxamido-2,2,2-trifluoroethyl, 2,6-dichlorobenzyl, 2-(3,5-dimethoxyphenyl)-2-propyloxycarbonyl, 2,4-dinitrophenyl, dithiasuccinyl, formyl, 4-methoxybenzenesulfonyl, 4-methoxybenzyl, 4-methylbenzyl, o-nitrophenylsulfenyl, 2-phenyl-2-propyloxycarbonyl, α-2,4,5-tetramethylbenzyloxycarbonyl, p-toluenesulfonyl, xanthenyl, benzyl ester, N-hydroxysuccinimide ester, p-nitrobenzyl ester, p-nitrophenyl ester, phenyl ester, p-nitrocarbonate, p-nitrobenzylcarbonate, trimethylsilyl and pentachlorophenyl ester.

The term “bioavailable” is art-recognized and refers to a form of the subject disclosure that allows for it, or a portion of the amount administered, to be absorbed by, incorporated to, or otherwise physiologically available to a subject or patient to whom it is administered.

### VII. Equivalents and Scope

Those skilled in the art will recognize or be able to ascertain using no more than routine experimentation, many equivalents to the specific embodiments in accordance with the disclosure described herein. The scope of the present disclosure is not intended to be limited to the foregoing Description, but rather is as set forth in the appended claims.

In the claims, articles such as “a,” “an,” and “the” mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process, unless indicated to the contrary or otherwise evident from the context. Throughout the Description and Claims, embodiments are provided in which exactly one member of the group is present in, employed in, or otherwise relevant to a given product or process. Throughout the Description and Claims, embodiments are provided in which more than one or all of the group members are present in, employed in, or otherwise relevant to a given product or process.

Throughout the Description and Claims, use of the term “comprising” is intended to be open and contemplates or permits the inclusion of additional elements or steps.

Where ranges are given, endpoints are included. Furthermore, it is to be understood that, unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure (e.g., any small molecule; any method of production; any method of use; etc.) can be excluded from any one or more claims, for any reason, whether or not related to the existence of prior art.

All cited sources, for example, references, publications, databases, database entries, and art cited herein, are incorporated into this application by reference, even if not expressly stated in the citation. In case of conflicting statements of a cited source and the instant application, the statement in the instant application shall control.

The disclosure is further illustrated by the following non-limiting examples. It is to be understood that the foregoing description and following examples are intended to illustrate and not limit the scope of the present disclosure, which is defined by the scope of the appended claims.

The present disclosure is further illustrated by the following non-limiting examples.

### EXAMPLES

### Example 1. Synthesis of the Compounds

The compounds of the disclosure may be prepared using any convenient methodology known to a person of the art. Nonlimiting synthetic methods for the compounds of the present disclosure are provided below. All other compounds of the present disclosure may be prepared with similar methods.

### Definition of Volume

Definition of volume of solvent: 1 volume means 1 g solute in 1 mL solvent and 10 volume means 1 g solute in 10 mL solvent.

### Synthesis of Compounds 100-115

### General Information:

### LCMS Analysis Condition:

Instrument name: Agilent Technologies 1290 infinity 11.

Method A: Method: A—0.1% TFA in H₂O, B—0.1% TFA in ACN; flow rate: 2.0 mL/min; column: XBridge C8 (50×4.6 mm, 3.5 μm) or BEH C8, +ve mode

Method B: Method: A—10 mM NH₄HCO₃ in H₂O, B—ACN; flow rate: 1.0 mL/min; column: XBridge C8 (50×4.6 mm, 3.5 μm) or BEH C8, +ve mode

Method C: Method: A—0.1% HCOOH in H₂O, B—0.1% FA in ACN; flow rate: 1.5 mL/min; column: ZORBAX XDB C-18 (50×4.6 mm, 3.5 μm) or BEH C8, +ve mode

Method D: Method: A—10 mM NH₄OAc in H₂O, B—ACN; flow rate: 1.0 mL/min; column: XBridge C8 (50×4.6 mm, 3.5 μm) or BEH C8, +ve mode

### HPLC Analysis Condition:

Instrument name: Agilent 1200 Series instruments as followed using % with UV detection (maxplot).

Method A: Method: A—0.1% TFA in H₂O, B—0.1% TFA in ACN; flow rate: 2.0 mL/min; column: XBridge C8 (50×4.6 mm, 3.5 μm).

Method B: Method: A—10 mM NH₄HCO₃ in H₂O, B-ACN; flow rate: 1.0 mL/min; column: XBridge C8 (50×4.6 mm, 3.5 μm).

### Prep-HPLC Purification Condition:

Method A: A—0.1% TFA in H₂O, B-MeOH or ACN; column: Sunfire C8 (19×250 mm, 5 μm) or Sunfire C18 (30×250 mm, 10 μm).

Method B: A—10 mM NH₄HCO₃ in H₂O, B-MeOH or ACN, Column: Sunfire C8 (19×250 mm, 5 μm) or Sunfire C18 (30×250 mm, 10 μm).

### Chiral SFC Purification Condition:

Instrument name: PIC-P10-20 (analytical)

Method A: Mobile Phase: 0.1% Isopropylamine in IPA:MeOH (1:1), flow rate: 3 mL/min; column: Lux A1 (250×4.6 mm, 5 μm).

Method B: Mobile Phase: 0.1% Isopropylamine in IPA:MeOH (1:1), flow rate: 3 mL/min; column: Chiralpak OX-H (250×4.6 mm, 5 μm).

Method C: Mobile Phase: 0.5% Isopropylamine in MeOH (1:1), flow rate: 3 mL/min; column: Lux C3 (250×4.6 mm, 5 μm).

### Chiral SFC Purification Condition:

Instrument name: PIC SFC 175

Method A: Mobile Phase: 0.1% Isopropylamine in IPA:MeOH (1:1), flow rate: 100 mL/min; column: Lux A1 (250×30 mm, 5 μm).

Method B: Mobile Phase: 0.1% Isopropylamine in IPA:MeOH (1:1), flow rate: 100 mL/min; column: Chiralpak OX-H (250×30 mm, 5 μm).

Method C: Mobile Phase: 0.5% Isopropylamine in IPA:MeOH (1:1), flow rate: 100 mL/min; column: Lux A1 (250×30 mm, 5 μm).

NMR instrument name: BRUKER NMR, model AV-II and AV-III 400 MHz FT-NMR.

### General Procedure (Scheme 100)

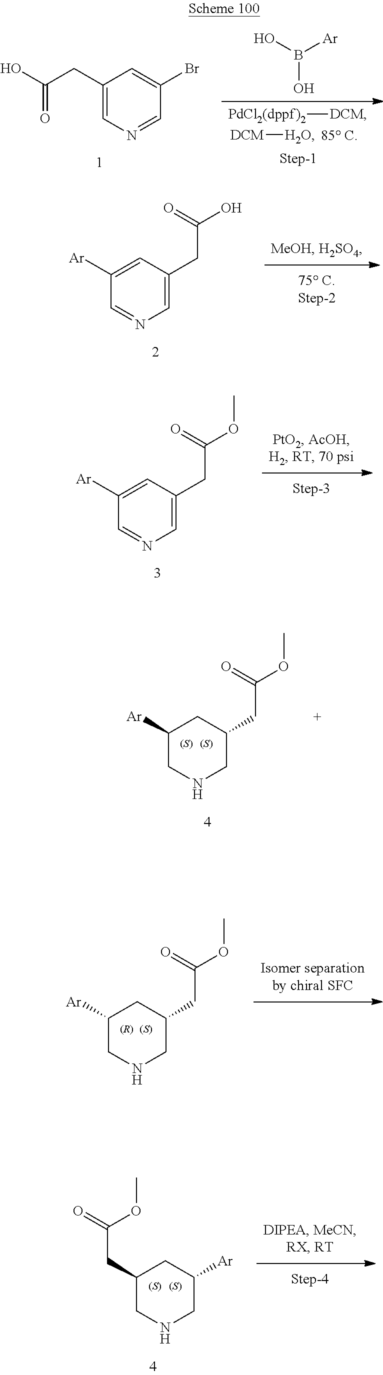

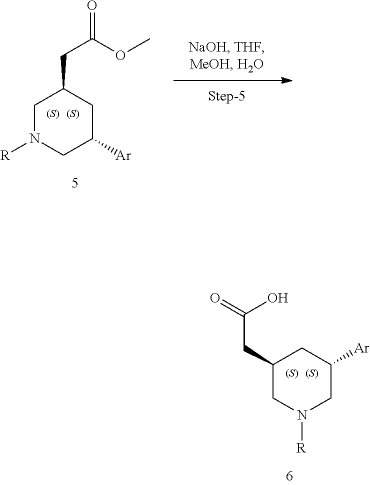

### Step 1: Suzuki Coupling.

To a stirred solution of compound 1 (1 equiv) in DME:water (10:1, 10 vol) at RT, arylboronic acid (1.1 equiv.), potassium carbonate (3.0 equiv), Pd(dppf)Cl₂·DCM (0.05 equiv) were added and the reaction mixture was purged with nitrogen (gas) for 1-15 min at RT. The reaction mixture was heated at 85° C. for 8-24 h. After completion (the reaction was monitored by LCMS), reaction mixture was concentrated under vacuum. The resulting crude residue was acidified with HCl in dioxane (4 M) and concentrated under vacuum to get the acid intermediate 2 which was used in the next step without further purification.

### Step 2: Esterification

To a stirred mixture of compound 2 (1 equiv) in MeOH (10 vol) was added conc. H₂SO₄ (3.2 equiv) and the reaction mixture was heated at 75° C. for 5-24 h. After completion (the reaction was monitored by LCMS), the reaction mixture was concentrated and was neutralized with 10% aq. NaHCO₃ solution. The resulting suspension was extracted with DCM (2×10 vol). The combined organic layer was washed with water (10 vol), brine (10 vol), dried over anhydrous sodium sulphate and the solvent was evaporated under vacuum. The crude residue was purified by column chromatography on Biotage Isolera (100-200 mesh silica gel eluting with 0-60% EtOAc in pet ether) to afford the ester compound 3.

### Step 3: Ring Reduction

To a stirred solution of compound 3 (1 equiv) in AcOH (10 vol) was added PtO₂ (0.28 equiv) and the reaction mixture was stirred under H₂ atmosphere (70 psi) for 16 h at RT. The reaction mixture was monitored by TLC. After completion (TLC showed starting material was consumed), the reaction mixture was concentrated under vacuum and the residue was neutralized with 10% aq. NaHCO₃ solution. The resulting suspension was extracted with EtOAc (2×10 vol). The combined organic layer was washed with water (10 vol), brine (10 vol), dried over anhydrous sodium sulphate and the solvent was evaporated under vacuum to get crude compound 4 which was used in next step without further purification.

### Step 4: Alkylation

To a stirred solution of compound 4 (1 equiv) in ACN (50 vol) was added DIPEA (3 equiv) and the reaction mixture was stirred at RT for 1-10 min followed by the addition of alkyl halide (1.2 equiv). The reaction mixture was stirred at RT for 6-24 h. After completion (monitored by TLC), the reaction mixture was diluted with water (100 vol) and extracted with EtOAc (3×100 vol). The combined organic layer was washed with water (50 vol), brine (50 vol), dried over anhydrous sodium sulphate and the solvent was evaporated under vacuum. The crude residue was purified by column chromatography on Biotage Isolera (100-200 mesh silica gel eluting with 0-10% EtOAc in pet ether) to afford compound 5 (the minor isomer was isolated as the desired anti isomer).

### Step 5: Ester Hydrolysis

To a stirred solution of compound 5 (1 equiv) in MeOH-THF-water (3:3:1, 10 vol) was added NaOH (3.0 equiv) at RT and the reaction mixture was stirred at RT for 1-12 h. After completion (the reaction mixture was monitored by TLC), the reaction mixture was concentrated under vacuum. The residue was acidified with aq. HCl (3 N) to pH 4-5. The precipitated solid was filtered, washed with water (twice) and pentane and then dried under vacuum to get the desired product. If the precipitation after acidification was not complete, then the resulting suspension was extracted with DCM (2×100 vol). The combined organic layer was washed with water (100 vol), brine (100 vol), dried over anhydrous sodium sulphate and concentrated under vacuum to get compound 6.

### 100

### Step 1: 2-(5-(4-(trifluoromethyl)phenyl)pyridin-3-yl)acetic acid

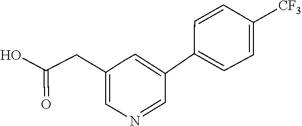 [SMILES: O=C(O)CC1=CN=CC(c2ccc(C(F)(F)F)cc2)=CC1]

2-(5-bromopyridin-3-yl)acetic acid (30 g, 139 mmol) and (4-(trifluoromethyl)phenyl)boronic acid (29.0 g, 153 mmol) were used to synthesize title compound using general procedure for Step 1. Yield: crude (39 g, brown solid). LCMS: (Method A) 281.9 (M+H), Rt. 1.97 min, 46.89% (Max).

### Step 2: methyl 2-(5-(4-(trifluoromethyl)phenyl)pyridin-3-yl)acetate

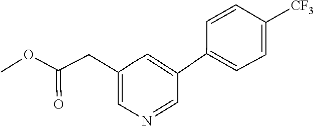 [SMILES: COC(=O)CC1=CN=CC(c2ccc(C(F)(F)F)cc2)=CC1]

2-(5-(4-(trifluoromethyl)phenyl)pyridin-3-yl)acetic acid (36.8 g, 375 mmol) was used to synthesize the title compound using general procedure Step 2 for esterification. Yield: 47% (19.7 g, light brown solid). ¹H NMR (400 MHz, DMSO-d₆): δ 8.87 (d, J=2.4 Hz, 1H), 8.56 (d, J=2.0 Hz, 1H), 8.10-8.09 (m, 1H), 7.97 (d, J=8.4 Hz, 2H), 7.88 (d, J=8.0 Hz, 2H), 3.87 (s, 2H), 3.66 (s, 3H). LCMS: (Method C) 296.1 (M+H), Rt. 2.08 min, 98.90% (Max).

### Step 3: methyl 2-(5-(4-(trifluoromethyl)phenyl)piperidin-3-yl)acetate

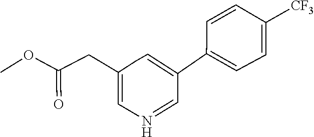 [SMILES: COC(=O)CC1=CN=CC(c2ccc(C(F)(F)F)cc2)=CC1]

Methyl 2-(5-(4-(trifluoromethyl)phenyl)pyridin-3-yl)acetate (3.4 g, 11.51 mmol) was used to synthesize the title compound (as mixture of stereoisomers) using general procedure step 3 for reduction. Yield: 98% (3.4 g, brown liquid). ¹H NMR (400 MHz, DMSO-d₆): δ 7.64 (d, J=8.4 Hz, 2H), 7.52-7.44 (m, 2H), 3.59-3.57 (m, 3H), 3.00-2.86 (m, 2H), 2.80-2.38 (m, 3H), 2.29-2.08 (m, 3H), 1.98-1.82 (m, 2H). LCMS: (Method C) 302.0 (M+H), Rt. 1.13 min, 99.68% (Max).

### Step 4: methyl 2-((anti)-1-(4-(trifluoromethyl)benzyl)-5-(4-(trifluoromethyl)phenyl)piperidin-3-yl)acetate and methyl 2-((syn)-1-(4-(trifluoromethyl)benzyl)-5-(4-(trifluoromethyl)phenyl)piperidin-3-yl)acetate

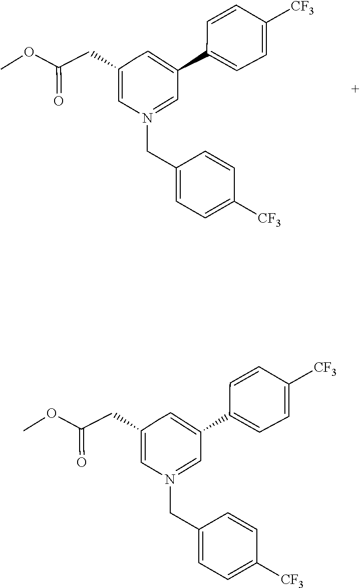

Methyl 2-(5-(4-(trifluoromethyl)phenyl)piperidin-3-yl)acetate (1.0 g, 3.31 mmol) and 1-(bromomethyl)-4-(trifluoromethyl)benzene (1.18 g, 4.97 mmol) were used to synthesize the title compounds using general procedure step 4 for alkylation.

Non-polar isomer (minor isomer, assigned as anti):

Yield: 19% (0.3 g, pale yellow liquid). ¹H NMR (400 MHz, DMSO-d₆): δ 7.69-7.64 (m, 4H), 7.57-7.53 (m, 4H), 3.66-3.62 (m, 1H), 3.54-3.51 (m, 4H), 3.15-3.09 (m, 1H), 2.82-2.15 (m, 7H), 1.81-1.62 (m, 2H). LCMS: (Method A) 460.0 (M+H), Rt. 2.53 min, 94.88% (Max).

HPLC: (Method A) Rt. 4.56 min, 99.82% (Max).

Polar isomer (major isomer, assigned as syn):

Yield: 46.05% (0.7 g, colorless liquid). LCMS: (Method A) 460.0 (M+H), Rt. 2.56 min, 98.34% (Max).

### Step 5: 2-((anti)-1-(4-(rifluoromethyl)benzyl)-5-(4-(trifluoromethyl)phenyl)piperidin-3-yl)acetic acid

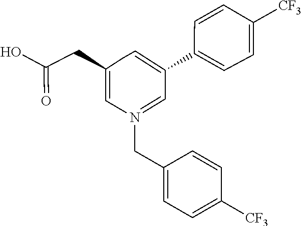

Methyl 2-((anti)-1-(4-(trifluoromethyl)benzyl)-5-(4-(trifluoromethyl)phenyl)piperidin-3-yl)acetate (0.25 g, 0.54 mmol) was used to synthesize the title compound using (anti, racemic) general procedure step 5 for ester hydrolysis. Yield: 82% (200 mg, white solid). ¹H NMR (400 MHz, DMSO-d₆): δ 12.05 (s, 1H), 7.69-7.64 (m, 4H), 7.59-7.54 (m, 4H), 3.65-3.61 (m, 1H), 3.57-3.53 (m, 1H), 3.15-3.08 (m, 1H), 2.76-2.73 (m, 1H), 2.60-2.34 (m, 5H), 2.22-2.13 (m, 1H), 1.82-1.79 (m, 1H), 1.74-1.67 (m, 1H). LCMS: (Method A) 446.0 (M+H), Rt. 2.45 min, 99.06% (Max). HPLC: (Method A) Rt. 4.27 min, 98.77% (Max).

### 101

### Step 1: Methyl 2-((3S,5S)-5-(4-(trifluoromethyl)phenyl)piperidin-3-yl)acetate

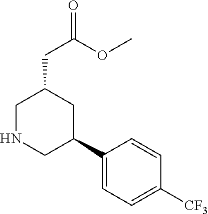 [SMILES: COC(=O)C[C@H]1CNC[C@H](c2ccc(C(F)(F)F)cc2)C1]

The title compound was synthesized by using methyl 2-(5-(4-(trifluoromethyl)phenyl)pyridin-3-yl)acetate (19.7 g, 66.76 mmol, three batches) as described in step 3 for ring reduction. Yield: 98% (combined yield 19.8 g, brown Oil). Isomers were separated by two successive Chiral SFC purifications. First purification by using Method A gave a mixture of two isomers as fraction 1. Yield: 48.2% (9.7 g, brown Oil). Chiral SFC: (Method B) Rt. 2.27 min, 34.20% (Max) and Rt. 3.08 min, 64.87% (Max). Fraction1 was further purified by Chiral SFC purification Method B to get the title compound as pure enantiomer. Yield: 14% (2.8 5 g, brown solid). LCMS: (Method C) 302.1 (M+H), Rt. 1.25 min, 96.04% (Max), Chiral SFC: (Method B) Rt. 2.35 min, 99.63% (Max).

### Step 2: Methyl 2-((3S,5S)-1-(4-(trifluoromethyl)benzyl)-5-(4-(trifluoromethyl)phenyl)piperidin-3-yl)acetate

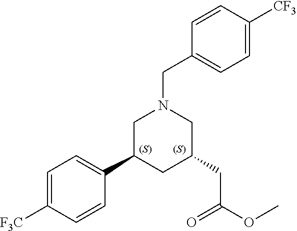

Methyl 2-((3S,5S)-5-(4-(trifluoromethyl)phenyl)piperidin-3-yl)acetate (120 mg, 0.39 mmol, Step-1 of Compound 101) and 1-(bromomethyl)-4-(trifluoromethyl)benzene (114 mg, 0.47 mmol) were used to synthesize the title compound using general procedure step 4. Yield: 70% (128 mg, colorless liquid). LCMS: (Method C) 460.1 (M+H), Rt. 2.10 min, 98.68% (Max).

### Step 3: 2-((3S,5S)-1-(4-(trifluoromethyl)benzyl)-5-(4-(trifluoromethyl)phenyl)piperidin-3-yl)acetic acid

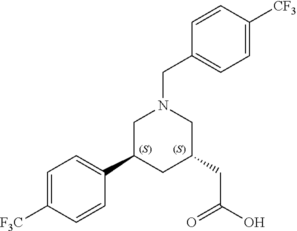

Methyl 2-((3S,5S)-1-(4-(trifluoromethyl)benzyl)-5-(4-(trifluoromethyl)phenyl)piperidin-3-yl)acetate (120 mg, 0.26 mmol) was used to synthesize the title compound using general procedure step 5. Yield: 69% (80 mg, off white solid). ¹H NMR (400 MHz, DMSO-d₆): δ 12.01 (s, 1H), 7.69-7.54 (m, 8H), 3.65-3.53 (m, 2H), 3.13-3.09 (m, 1H), 2.76-2.68 (m, 1H), 2.47-2.33 (m, 5H), 2.23-2.12 (m, 1H), 1.81-1.74 (m, 1H), 1.70-1.65 (m, 1H). LCMS: (Method D) 446.1 (M+H), Rt. 2.24 min, 96.90% (Max). HPLC: (Method A) Rt. 4.17 min, 99.94% (Max). Chiral SFC: (Method C) Rt. 2.55 min, 99.74% (Max).

### 102

### Step 1: 2-((3R,5R)-1-(4-(trifluoromethyl)benzyl)-5-(4-(trifluoromethyl)phenyl)piperidin-3-yl)acetic acid

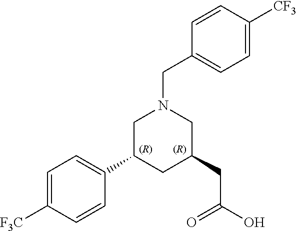

Methyl 2-((3R,5R)-5-(4-(trifluoromethyl)phenyl)piperidin-3-yl)acetate (peak at 5.65 min from the SFC purification method A) (0.1 g, 0.33 mmol) was used to synthesize the title compound using general procedure step 5. Yield: 39% (38.2 mg, off white solid). ¹H NMR (400 MHz, DMSO-d₆): δ 12.05 (s, 1H), 7.69-7.64 (m, 4H), 7.59-7.54 (m, 4H), 3.65-3.53 (m, 2H), 3.16-3.08 (m, 1H), 2.76-2.67 (m, 1H), 2.45-2.30 (m, 5H), 2.20-2.12 (m, 1H), 1.82-1.72 (m, 1H), 1.72-1.60 (m, 1H). LCMS: (Method C) 446.1 (M+H), Rt. 1.61 min, 99.10% (Max). HPLC: (Method A) Rt. 4.24 min, 99.85% (Max). Chiral SFC: (Method C) Rt. 1.96 min, 99.74% (Max).

### 103

### Step 1: 2-(5-(3-methoxyphenyl)pyridin-3-yl)acetic acid

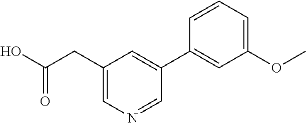 [SMILES: COc1cccc(-c2cncc(CC(=O)O)c2)c1]

2-(5-bromopyridin-3-yl)acetic acid (2.0 g, 9.25 mmol) and (3-methoxyphenyl)boronic acid (1.54 g, 10.18 mmol) were used to synthesize the title compound by using general procedure step 1. Yield: crude (2.25 g, grey solid). LCMS: (Method C) 244.0 (M+H), Rt. 0.96 min, 71.95% (Max).

### Step 2: methyl 2-(5-(3-methoxyphenyl)pyridin-3-yl)acetate

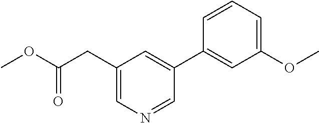

2-(5-(3-Methoxyphenyl)pyridin-3-yl)acetic acid (2.25 g, 9.24 mmol) was used to synthesize the title compound by using general procedure step 2. Yield: 84% (2.0 g, brown liquid). ¹H NMR (300 MHz, DMSO-d₆): δ 8.80 (d, J=2.4 Hz, 1H), 8.48 (d, J=2.1 Hz, 1H), 8.01-7.99 (m, 1H), 7.45-7.40 (m, 1H), 7.29-7.24 (m, 2H), 7.02-6.98 (m, 1H), 3.84 (s, 5H), 3.65 (s, 3H). LCMS: (Method C) 258.1 (M+H), Rt. 1.42 min, 94.98% (Max).

### Step 3: methyl 2-(5-(3-methoxyphenyl)piperidin-3-yl)acetate

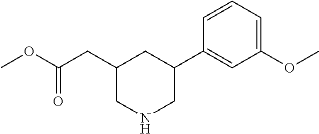

Methyl 2-(5-(3-methoxyphenyl)pyridin-3-yl)acetate (2.0 g, 7.77 mmol) was used to synthesize the title compound using general procedure step 3. Yield: 88% (1.8 g, brown liquid). LCMS: (Method C) 264.1 (M+H), Rt. 1.00 min, 94.90% (Max).

### Step 4: methyl 2-((anti)-5-(3-methoxyphenyl)-1-(4-(rifluoromethyl)benzyl)piperidin-3-yl)acetate and methyl 2-((syn)-5-(3-methoxyphenyl)-1-(4-(trifluoromethyl)benzyl)piperidin-3-yl)acetate

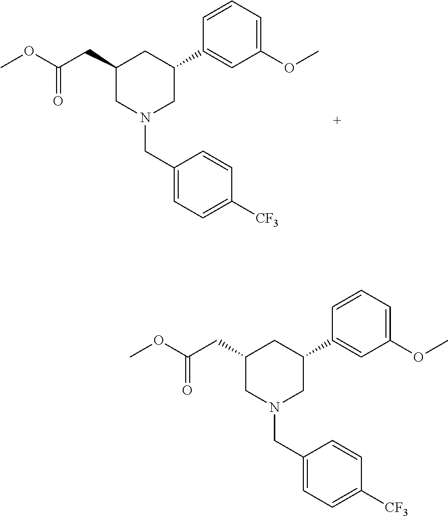

Methyl 2-(5-(3-methoxyphenyl)piperidin-3-yl)acetate (1.8 g, 6.83 mmol) and 1-(bromomethyl)-4-(trifluoromethyl)benzene (2.45 g, 10.25 mmol) were used to synthesize the title compound using general procedure step 4.

Non-polar isomer (minor isomer, assigned as anti):

Yield: 15% (0.35 g, pale yellow liquid). ¹H NMR (400 MHz, DMSO-d₆): δ 7.69 (d, J=8.0 Hz, 2H), 7.54 (d, J=8.0 Hz, 2H), 7.20-7.16 (m, 1H), 6.80-6.75 (m, 3H), 3.72 (s, 3H), 3.61 (s, 2H), 3.56 (s, 3H), 2.88-2.75 (m, 4H), 2.30-2.18 (m, 2H), 2.18-2.05 (m, 1H), 2.05-1.95 (m, 1H), 1.92-1.80 (m, 1H), 1.80-1.60 (m, 1H). LCMS: (Method A) 422.1 (M+H), Rt. 1.95 min, 96.85% (Max). HPLC: (Method A) Rt. 4.00 min, 97.74% (Max).

Polar isomer (major isomer, assigned as syn):

Yield: 66% (1.5 g, colorless liquid). LCMS: (Method A) 422.2 (M+H), Rt. 1.93 min, 96.98% (Max).

### Step 5: 2-((anti)-5-(3-methoxyphenyl)-1-(4-(trifluoromethyl)benzyl)piperidin-3-yl)acetic acid

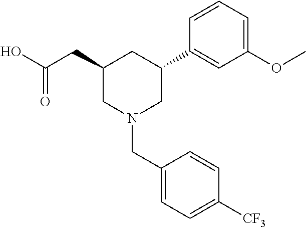 [SMILES: COc1cccc([C@H]2C[C@H](CC(=O)O)CN(Cc3ccc(C(F)(F)F)cc3)C2)c1]

Methyl 2-((anti)-5-(3-methoxyphenyl)-1-(4-(trifluoromethyl)benzyl)piperidin-3-yl)acetate (0.15 g, 0.36 mmol) was used to synthesize the title compound (anti, racemic) using general procedure step 5. Yield: 39% (59 mg, white solid). ¹H NMR (400 MHz, DMSO-d₆): δ 12.10 (s, 1H), 7.68 (d, J=8.0 Hz, 2H), 7.54 (d, J=8.0 Hz, 2H), 7.21-7.17 (m, 1H), 6.88-6.85 (m, 2H), 6.77-6.74 (m, 1H), 3.73 (s, 3H), 3.62 (d, J=14.0 Hz, 1H), 3.52 (d, J=14.0 Hz, 1H), 2.98-2.90 (m, 1H), 2.80-2.70 (m, 1H), 2.62-2.15 (m, 6H), 1.78-1.69 (m, 1H), 1.69-1.62 (m, 1H). LCMS: (Method A) 408.0 (M+H), Rt. 2.27 min, 96.96% (Max). HPLC: (Method A) Rt. 3.73 min, 95.41% (Max).

### 104

### Step 1: 2-(5-(3-(trifluoromethyl)phenyl)pyridin-3-yl)acetic acid

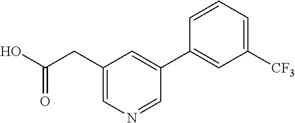 [SMILES: O=C(O)Cc1cncc(-c2cccc(C(F)(F)F)c2)c1]

2-(5-Bromopyridin-3-yl)acetic acid (1.5 g, 6.94 mmol) and (3-(trifluoromethyl)phenyl)boronic acid (1.45 g, 7.63 mmol) were used to synthesize the title compound using general procedure step 1. Yield: crude (1.95 g, brown solid). LCMS: (Method C) 282.0 (M+H), Rt. 1.53 min, 63.87% (Max).

### Step 2: methyl 2-(5-(3-(trifluoromethyl)phenyl)pyridin-3-yl)acetate

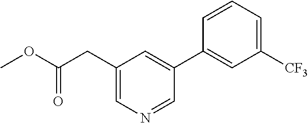 [SMILES: COC(=O)Cc1cncc(-c2cccc(C(F)(F)F)c2)c1]

2-(5-(3-(Trifluoromethyl)phenyl)pyridin-3-yl)acetic acid (1.95 g, 6.9 mmol) was used to synthesize the title compound using general procedure step 2. Yield: 73% (1.5 g, brown liquid). LCMS: (Method A) 295.9 (M+H), Rt. 2.13 min, 98.35% (Max).

### Step 3: methyl 2-(5-(3-(trifluoromethyl)phenyl)piperidin-3-yl)acetate

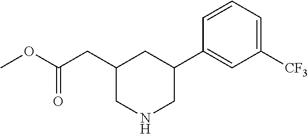 [SMILES: COC(=O)CC1CNCC(c2cccc(C(F)(F)F)c2)C1]

Methyl 2-(5-(3-(trifluoromethyl)phenyl)pyridin-3-yl)acetate (1.2 g, 4.06 mmol) was used to synthesize the title compound using general procedure step 3. Yield: 77% (0.95 g, brown liquid). LCMS: (Method D) 302.5 (M+H), Rt. 1.79 min, 99.22% (Max).

### Step 4: methyl 2-((anti)-1-(4-(trifluoromethyl)benzyl)-5-(3-(trifluoromethyl)phenyl)piperidin-3-yl)acetate and methyl 2-((syn)-1-(4-(trifluoromethyl)benzyl)-5-(3-(trifluoromethyl)phenyl)piperidin-3-yl)acetate

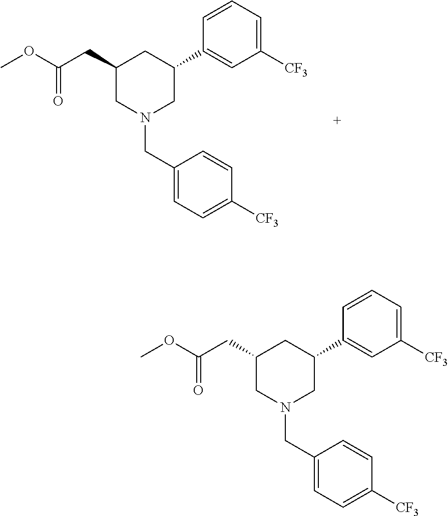 [SMILES: COC(=O)C[C@H]1C[C@H](c2cccc(C(F)(F)F)c2)CN(Cc2ccc(C(F)(F)F)cc2)C1 ; COC(=O)C[C@@H]1C[C@H](c2cccc(C(F)(F)F)c2)CN(Cc2ccc(C(F)(F)F)cc2)C1]

Methyl 2-(5-(3-(trifluoromethyl)phenyl)piperidin-3-yl)acetate (0.95 g, 3.15 mmol) and 1-(bromomethyl)-4-(trifluoromethyl)benzene (1.13 g, 4.73 mmol) were used to synthesize the title compound using general procedure step 4.

Non-polar isomer (minor isomer, assigned as anti):

Yield: 21% (0.3 g, colorless liquid). ¹H NMR (400 MHz, DMSO-d₆): δ 7.73-7.64 (m, 4H), 7.57-7.51 (m, 4H), 3.68-3.64 (m, 1H), 3.52-3.48 (m, 4H), 3.17-3.09 (m, 1H), 2.74-2.72 (m, 1H), 2.68-2.59 (m, 2H), 2.50-2.34 (m, 3H), 2.20-2.10 (m, 1H), 1.85-1.78 (m, 1H), 1.68-1.63 (m, 1H). LCMS: (Method D) 460.6 (M+H), Rt. 2.80 min, 99.89% (Max). HPLC: (Method A) Rt. 4.44 min, 99.50% (Max).

Polar isomer (major isomer, assigned as syn):

Yield: 38% (0.55 g, pale yellow liquid). LCMS: (Method C) 460.1 (M+H), Rt. 1.62 min, 99.73% (Max).

### Step 5: 2-((anti)-1-(4-(trifluoromethyl)benzyl)-5-(3-(trifluoromethyl)phenyl)piperidin-3-yl)acetic acid

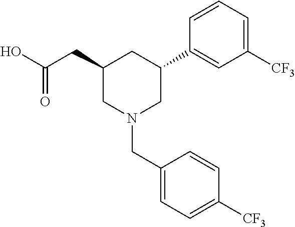 [SMILES: O=C(O)C[C@H]1C[C@H](c2cccc(C(F)(F)F)c2)CN(Cc2ccc(C(F)(F)F)cc2)C1]

Methyl 2-((anti)-1-(4-(trifluoromethyl)benzyl)-5-(3-(trifluoromethyl)phenyl)piperidin-3-yl)acetate (0.3 g, 0.65 mmol) was used to synthesize the title compound (anti, racemic) using general procedure step 5. Yield: 72% (210 mg, white solid). ¹H NMR (400 MHz, DMSO-d₆): δ 12.03 (s, 1H), 7.75 (s, 1H), 7.68-7.65 (m, 3H), 7.55-7.50 (m, 4H), 3.65 (d, J=14.0 Hz, 1H), 3.52 (d, J=14.0 Hz, 1H), 3.18-3.09 (m, 1H), 2.75-2.65 (m, 1H), 2.50-2.30 (m, 5H), 2.20-2.05 (m, 1H), 1.85-1.75 (m, 1H), 1.70-1.60 (m, 1H). LCMS: (Method A) 446.0 (M+H), Rt. 2.45 min, 96.50% (Max). HPLC: (Method A) Rt. 4.16 min, 97.59/6 (Max).

### 105

### Step 1: 2-(5-(4-methoxyphenyl)pyridin-3-yl)acetic acid

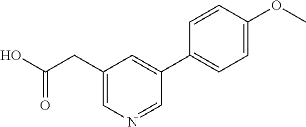 [SMILES: COc1ccc(-c2cncc(CC(=O)O)c2)cc1]

2-(5-Bromopyridin-3-yl)acetic acid (2.0 g, 9.25 mmol) and (4-methoxyphenyl)boronic acid (1.5 g, 10.18 mmol) were used to synthesize the title compound using general procedure step 1. Yield: crude (2.25 g, brown solid). LCMS: (Method A) 244.0 (M+H), Rt. 1.24 min, 80.53% (Max).

### Step 2: methyl 2-(5-(4-methoxyphenyl)pyridin-3-yl)acetate

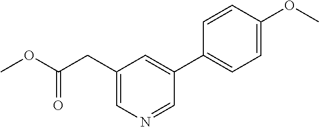

2-(5-(4-Methoxyphenyl)pyridin-3-yl)acetic acid (2.25 g, 9.24 mmol) was used to synthesize the title compound using general procedure step 2. Yield: 71% (1.7 g, pale yellow solid). ¹H NMR (400 MHz, DMSO-d₆): δ 8.76 (d, J=2.4 Hz, 1H), 8.42 (d, J=2.0 Hz, 1H), 7.95-7.93 (m, 1H), 7.68-7.66 (m, 2H), 7.09-7.06 (m, 2H), 3.83-3.82 (m, 5H), 3.66 (s, 3H). LCMS: (Method A) 258.3 (M+H), Rt. 1.50 min, 99.74% (Max).

### Step 3: methyl 2-(5-(4-methoxyphenyl)piperidin-3-yl)acetate

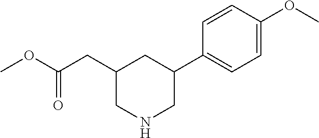

Methyl 2-(5-(4-methoxyphenyl)pyridin-3-yl)acetate (1.5 g, 5.82 mmol) was used to synthesize the title compound using general procedure step 3. Yield: 71% (1.1 g, brown liquid). LCMS: (Method C) 264.1 (M+H), Rt. 1.15 min, 97.60% (Max).

### Step 4: methyl 2-((anti)-5-(4-methoxyphenyl)-1-(4-(trifluoromethyl)benzyl)piperidin-3-yl)acetate and methyl 2-((syn)-5-(4-methoxyphenyl)-1-(4-(trifluoromethyl)benzyl)piperidin-3-yl)acetate

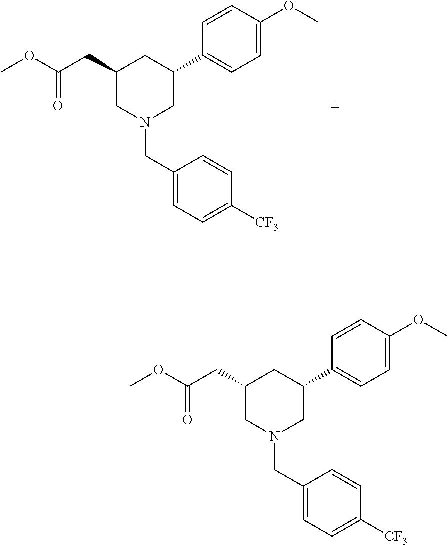

Methyl 2-(5-(4-methoxyphenyl)piperidin-3-yl)acetate (1.0 g, 3.7 mmol) and 1-(bromomethyl)-4-(trifluoromethyl)benzene (1.36 g, 5.6 mmol) were used to synthesize the title compound using general procedure step 4.

Non-polar isomer (minor isomer, assigned as anti):

Yield: 17.5% (0.280 g, white solid). ¹H NMR (400 MHz, DMSO-d₆): δ 7.68 (d, J=8.0 Hz, 2H), 7.53 (d, J=8.0 Hz, 2H), 7.20 (d, J=8.8 Hz, 2H), 6.85-6.83 (m, 2H), 3.71 (s, 3H), 3.63-3.60 (m, 1H), 3.55-3.45 (m, 4H), 3.00-2.85 (m, 1H), 2.80-2.65 (m, 2H), 2.50-2.40 (m, 2H), 2.30-2.12 (m, 3H), 1.75-1.58 (m, 2H). LCMS: (Method A) 422.1 (M+H), Rt. 1.89 min, 99.91% (Max). HPLC: (Method A) Rt. 3.95 min, 99.91% (Max).

Polar isomer (major isomer, assigned as syn):

Yield: 47% (0.75 g, colorless liquid). LCMS: (Method A) 422.1 (M+H), Rt. 1.99 min, 99.63% (Max).

### Step 5: 2-((anti)-5-(4-methoxyphenyl)-1-(4-(trifluoromethyl)benzyl)piperidin-3-yl)acetic acid

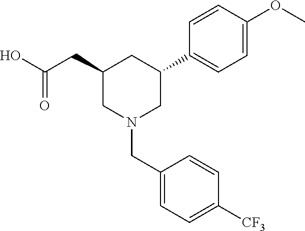 [SMILES: COc1ccc([C@H]2C[C@H](CC(=O)O)CN(Cc3ccc(C(F)(F)F)cc3)C2)cc1]

Methyl 2-((anti)-5-(4-methoxyphenyl)-1-(4-(trifluoromethyl)benzyl)piperidin-3-yl)acetate (0.15 g, 0.35 mmol) used to synthesize the title compound (anti, racemic) using general procedure step 5. Yield: 83% (0.12 g, white solid). ¹H NMR (400 MHz, CDCl₃): δ 7.65 (d, J=8.4 Hz, 2H), 7.56 (t, J=8.0 Hz, 2H), 7.15 (d, J=8.8 Hz, 2H), 6.88-6.84 (m, 2H), 3.98-3.89 (s, 2H), 3.80 (s, 3H), 3.32-3.08 (m, 4H), 2.72-2.50 (m, 3H), 2.35-2.28 (m, 1H), 1.95-1.80 (m, 2H). LCMS: (Method A) 408.0 (M+H), Rt. 2.26 min, 98.85% (Max). HPLC: (Method A) Rt. 3.71 min, 99.13% (Max).

### 106

### Step 1: 2-(5-(3-methoxy-5-(trifluoromethyl)phenyl)pyridin-3-yl)acetic acid

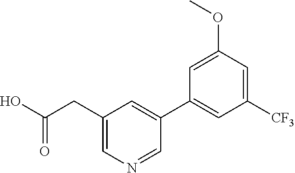 [SMILES: COc1cc(-c2cncc(CC(=O)O)c2)cc(C(F)(F)F)c1]

2-(5-Bromopyridin-3-yl)acetic acid (1.5 g, 6.94 mmol) and (3-methoxy-5-(trifluoromethyl)phenyl)boronic acid (1.67 g, 7.63 mmol) were used to synthesize the title compound using general procedure step 1. Yield: crude (2.16 g, brown solid). LCMS: (Method A) 312.0 (M+H), Rt. 1.63 min, 63.22% (Max).

### Step 2: methyl 2-(5-(3-methoxy-5-(trifluoromethyl)phenyl)pyridin-3-yl)acetate

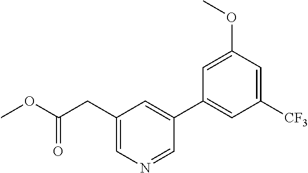 [SMILES: COC(=O)Cc1cncc(-c2cc(OC)cc(C(F)(F)F)c2)c1]

2-(5-(3-Methoxy-5-(trifluoromethyl)phenyl)pyridin-3-yl)acetic acid (2.16 g, 6.9 mmol) was used to synthesize the title compound using general procedure step 2. Yield: 66% (1.5 g, brown liquid). ¹H NMR (400 MHz, DMSO-d₆): δ 8.90 (d, J=2.0 Hz, 1H), 8.54 (d, J=2.0 Hz, 1H), 8.14-8.13 (m, 1H), 7.62-7.60 (m, 2H), 7.31 (s, 1H), 3.93 (s, 3H), 3.86 (s, 2H), 3.66 (s, 3H). LCMS: (Method C) 326.0 (M+H), Rt. 2.04 min, 99.72% (Max).

### Step 3: methyl 2-(5-(3-methoxy-5-(trifluoromethyl)phenyl)piperidin-3-yl)acetate

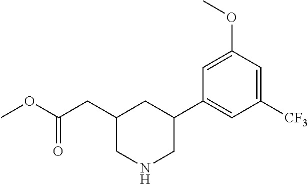 [SMILES: COC(=O)CC1CNCC(c2cc(OC)cc(C(F)(F)F)c2)C1]

Methyl 2-(5-(3-methoxy-5-(trifluoromethyl)phenyl)pyridin-3-yl)acetate (1.5 g, 4.61 mmol) was used to synthesize the title compound using general procedure step 3. Yield: 69% (1.1 g, pale yellow liquid). LCMS: (Method D) 332.5 (M+H), Rt. 1.88 min, 96.24% (Max).

### Step 4: methyl 2-((anti)-5-(3-methoxy-5-(trifluoromethyl)phenyl)-1-(4-(trifluoromethyl)benzyl)piperidin-3-yl)acetate and methyl 2-((syn)-5-(3-methoxy-5-(trifluoromethyl)phenyl)-1-(4-(trifluoromethyl)benzyl)piperidin-3-yl)acetate

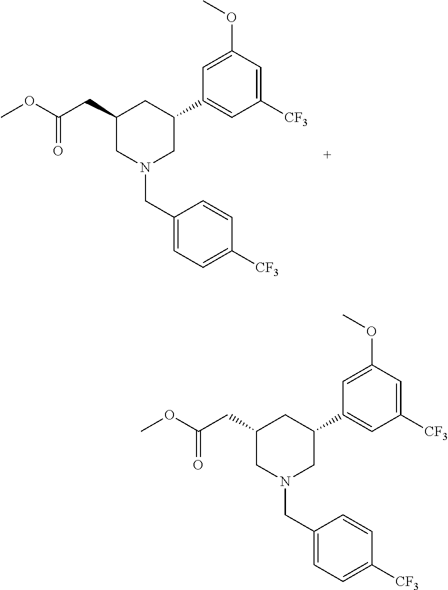 [SMILES: COC(=O)C[C@H]1C[C@H](c2cc(OC)cc(C(F)(F)F)c2)CN(Cc2ccc(C(F)(F)F)cc2)C1 ; COC(=O)C[C@@H]1C[C@H](c2cc(OC)cc(C(F)(F)F)c2)CN(Cc2ccc(C(F)(F)F)cc2)C1]

Methyl 2-(5-(3-methoxy-5-(trifluoromethyl)phenyl)piperidin-3-yl)acetate (1.1 g, 3.32 mmol) and 1-(bromomethyl)-4-(trifluoromethyl)benzene (1.19 g, 4.98 mmol) were used to synthesize the title compound using general procedure step 4.

Non-polar isomer (minor isomer, assigned as anti):

Yield: 19% (0.33 g, colorless liquid). ¹H NMR (400 MHz, DMSO-d₆): δ 7.67 (d, J=8.0 Hz, 2H), 7.53 (d, J=8.0 Hz, 2H), 7.29 (s, 1H), 7.20 (s, 1H), 7.06 (s, 1H), 3.82 (s, 3H), 3.66 (d, J=14.0 Hz, 1H), 3.52-3.47 (m, 4H), 3.12-3.08 (m, 1H), 2.73-2.67 (m, 2H), 2.51-2.30 (m, 4H), 2.20-2.10 (m, 1H), 1.84-1.78 (m, 1H), 1.68-1.60 (m, 1H). LCMS: (Method C) 490.1 (M+H), Rt. 2.09 min, 96.09% (Max). HPLC: (Method A) Rt. 3.87 min, 95.87% (Max).

Polar isomer (major isomer, assigned as syn):

Yield: 39% (0.65 g, pale yellow liquid). LCMS: (Method C) 490.1 (M+H), Rt. 1.69 min, 83.15% (Max).

### Step 5: 2-((anti)-5-(3-methoxy-5-(trifluoromethyl)phenyl)-1-(4-(trifluoromethyl)benzyl)piperidin-3-yl)acetic acid

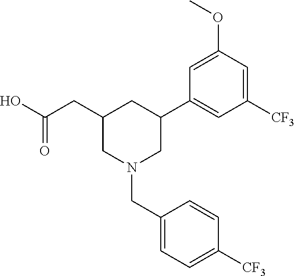 [SMILES: COc1cc(C2CC(CC(=O)O)CN(Cc3ccc(C(F)(F)F)cc3)C2)cc(C(F)(F)F)c1]

Methyl 2-((anti)-5-(3-methoxy-5-(trifluoromethyl)phenyl)-1-(4-(trifluoromethyl)benzyl)piperidin-3-yl)acetate (0.3 g, 0.61 mmol) was used to synthesize the title compound (anti, racemic) using general procedure step 5. Yield: 50% (145 mg, off white solid). ¹H NMR (400 MHz, CDCl₃): δ 7.61 (d, J=8.0 Hz, 2H), 7.49 (d, J=8.0 Hz, 2H), 7.13 (s, 1H), 6.99 (d, J=5.6 Hz, 2H), 3.84 (s, 3H), 3.73-3.63 (m, 2H), 3.20-3.14 (m, 1H), 3.00-2.94 (m, 1H), 2.83-2.78 (m, 2H), 2.70-2.60 (m, 1H), 2.58-2.48 (m, 1H), 2.45-2.32 (m, 2H), 1.92-1.80 (m, 2H). LCMS: (Method A) 476.0 (M+H), Rt. 2.48 min, 99.60% (Max). HPLC: (Method A) Rt. 4.31 min, 99.27% (Max).

### 107

### Step 1: Methyl 2-((anti)-5-(4-hydroxyphenyl)-1-(4-(trifluoromethyl)benzyl)piperidin-3-yl)acetate

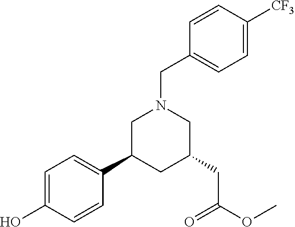 [SMILES: COC(=O)C[C@@H]1C[C@@H](c2ccc(O)cc2)CN(Cc2ccc(C(F)(F)F)cc2)C1]

To a stirred solution of methyl 2-((anti)-5-(4-methoxyphenyl)-1-(4-(trifluoromethyl)benzyl)piperidin-3-yl)acetate (560 mg, 1.33 mmol, Step-04 of Compound 105) in DCM (20 mL) at −78° C. was added BBr₃ (13.3 mL, 13.30 mmol, 1 M in DCM) and the reaction mixture was stirred at −78° C. for 3 h. The reaction mixture was then warmed to RT and then stirred until completion. After completion (monitored by TLC), the reaction mixture was cooled to −10° C. and quenched by dropwise addition of 10% aq. NaHCO₃ solution. The reaction mixture was stirred for 30 min at RT and then extracted with DCM (3×20 mL). The combined organic layer was washed with water (20 mL), brine (20 mL), dried over anhydrous Na₂SO₄ and the solvent was evaporated under vacuum to get the title compound which was used directly for next step. Yield: 74% (0.4 g, gummy solid). ¹H NMR (400 MHz, DMSO-d₆): δ 9.16 (d, J=5.4 Hz, 1H), 7.70-7.63 (m, 2H), 7.57-7.53 (m, 2H), 7.12-7.05 (m, 2H), 6.77-6.66 (m, 2H), 3.68-3.49 (m, 5H), 2.93-2.08 (m, 8H), 1.78-1.62 (m, 2H). LCMS: (Method C) 408.1 (M+H), Rt. 1.41 min, 90.39% (Max).

### Step 2: Methyl 2-((anti)-1-(4-(trifluoromethyl)benzyl)-5-(4-(((trifluoromethyl)sulfonyl)oxy)phenyl)piperidin-3-yl)acetate

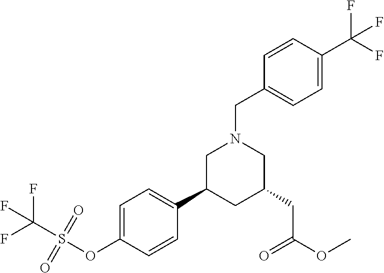 [SMILES: COC(=O)C[C@@H]1C[C@@H](c2ccc(OS(=O)(=O)C(F)(F)F)cc2)CN(Cc2ccc(C(F)(F)F)cc2)C1]

To a stirred solution of methyl 2-((anti)-5-(4-hydroxyphenyl)-1-(4-(trifluoromethyl)benzyl)piperidin-3-yl)acetate (0.1 g, 0.24 mmol) in DCM (5 mL) at RT were added DIPEA (0.085 mL, 0.49 mmol) followed by trifluoromethanesulfonic anhydride (83.14 mg, 0.29 mmol) and the reaction mixture was stirred at RT for 4 h. The reaction mixture was monitored by TLC. After completion, the reaction mixture was diluted with DCM and water. The resulting suspension was extracted with DCM (3×15 mL). The combined organic layer was washed with water (20 mL), brine (20 mL), dried over anhydrous sodium sulphate and the solvent was evaporated under vacuum to get the title compound which was used in the next step without further purification. Yield: 91% (120 mg, gummy solid). LCMS: (Method D) 540.0 (M+H), Rt. 2.64 min, 52.23.% (Max).

### Step 3: Synthesis of Methyl 2-((anti)-5-(4-cyanophenyl)-1-(4-(trifluoromethyl)benzyl)piperidin-3-yl)acetate

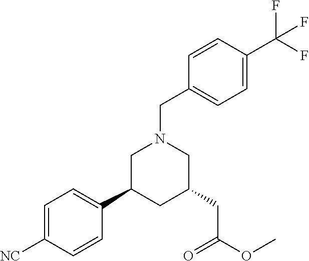 [SMILES: COC(=O)C[C@@H]1C[C@@H](c2ccc(C#N)cc2)CN(Cc2ccc(C(F)(F)F)cc2)C1]

To a stirred solution of methyl 2-((anti)-1-(4-(trifluoromethyl)benzyl)-5-(4-(((trifluoromethyl)sulfonyl)oxy)phenyl)piperidin-3-yl)acetate (120 mg, 0.23 mmol) in DMF (3 mL) were added zinc cyanide (21.0 mg, 0.178 mmol), Pd(PPh₃)₄ (26.0 mg, 0.02 mmol) at RT and the reaction mixture was purged with nitrogen gas for 5 min. The reaction mixture was heated at 80° C. for 16 h. After completion (the reaction was monitored by LCMS), the reaction mixture was diluted with water and extracted with DCM (3×15 mL). The combined organic layer was washed with water (20 mL), brine (20 mL), dried over anhydrous sodium sulphate, filtered, and solvent was evaporated under vacuum. The crude residue was purified by Prep HPLC (Method A). The prep fraction was concentrated. To the residual aqueous phase was added DCM and the mixture was neutralized with 10% aq. NaHCO₃ solution. The organic phase was separated, washed with water, brine, dried over anhydrous sodium sulphate and concentrated to afford the title compound. Yield: 16% (15 mg, gummy solid). LCMS: (Method A) 417.0 (M+H), Rt. 2.08 min, 92.48% (Max).

### Step 4: Synthesis of 2-((anti)-5-(4-cyanophenyl)-1-(4-(rifluoromethyl)benzyl)piperidin-3-yl)acetic acid

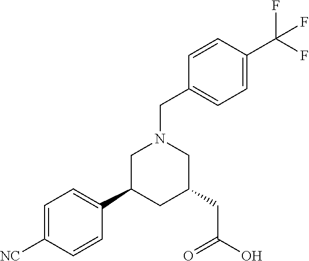 [SMILES: N#Cc1ccc([C@@H]2C[C@@H](CC(=O)O)CN(Cc3ccc(C(F)(F)F)cc3)C2)cc1]

To a stirred solution of methyl 2-((anti)-5-(4-cyanophenyl)-1-(4-(trifluoromethyl)benzyl)piperidin-3-yl)acetate (10 mg, 0.02 mmol) in a mixture of MeOH-THF-water (1 mL, 3:3:1) at RT was added LiOH·H₂O (1.5 mg, 0.03 mmol) and the reaction mixture was stirred at RT for 4 h. The reaction mixture was monitored by TLC. After completion, the reaction mixture was concentrated under vacuum and the residue was acidified with HCl solution (1.5 N). The resulting suspension was extracted with DCM (2×15 mL). The combined organic layer was washed with brine (10 mL), water (10 mL), dried over anhydrous sodium sulphate and concentrated under vacuum to get the title compound. Yield: 63% (6.0 mg, Off white solid). ¹H NMR (400 MHz, DMSO-d₆): δ 11.98 (br s, 1H), 7.74 (d, J=8.0 Hz, 2H), 7.68 (d, J=8.0 Hz, 2H), 7.58-7.52 (m, 4H), 3.62-3.51 (m, 2H), 3.32-3.07 (m, 2H), 2.72-2.11 (m, 6H), 1.79-1.71 (m, 1H), 1.67-1.61 (m, 1H). LCMS: (Method A) 403.0 (M+H), Rt. 2.23 min, 94.15.% (Max). HPLC: (Method A) Rt. 3.43 min, 96.94% (Max).

### 108

### Step 1: Methyl 2-((3S,5S)-1-(3-methoxy-5-(trifluoromethyl)benzyl)-5-(4-(trifluoromethyl)phenyl)piperidin-3-yl)acetate

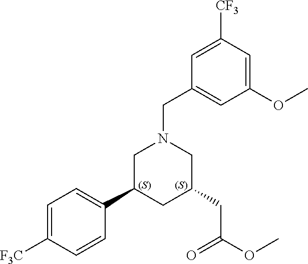

The title compound was synthesized by using methyl 2-((3S,5S)-5-(4-(trifluoromethyl)phenyl)piperidin-3-yl)acetate (100 mg, 0.33 mmol, Step-1 of Compound 101), in ACN (4 mL), DIPEA (0.17 mL, 0.99 mmol), 1-(bromomethyl)-3-methoxy-5-(trifluoromethyl)benzene (107.2 mg, 0.39 mmol) as described in step 4 for alkylation. Yield: 80% (130 mg, gummy solid). ¹H NMR (300 MHz, DMSO-d₆): δ 7.65-7.55 (m, 4H), 7.21-7.10 (m, 3H), 3.84 (s, 3H), 3.79-3.68 (m, 1H), 3.57 (s, 3H), 3.45-3.49 (m, 1H), 3.32-3.09 (m, 2H), 2.78-2.20 (m, 6H), 1.80-1.75 (m, 1H), 1.68-1.63 (m, 1H). LCMS: (Method C) 490.1 (M+H), Rt. 2.20 min, 99.93% (Max).

### Step 2: 2-((1S,5S)-3-(3-methoxy-5-(trifluoromethyl)benzyl)-5-(4-(trifluoromethyl)phenyl)cyclohexyl)acetic acid

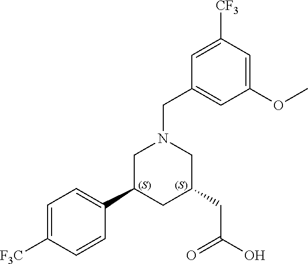

The title compound was synthesized by using methyl 2-((3S,5S)-1-(3-methoxy-5-(trifluoromethyl)benzyl)-5-(4-(trifluoromethyl)phenyl)piperidin-3-yl)acetate (120 mg, 0.24 mmol) as described in step 5 for ester hydrolysis. Yield: 41% (48 mg, Off white solid). ¹H NMR (400 MHz, CD₃OD): δ 7.59 (d, J=8.0 Hz, 2H), 7.53 (d, J=8.4 Hz, 2H), 7.25-7.22 (m, 2H), 7.08 (s, 1H), 3.87 (s, 3H), 3.75 (d, J=13.6 Hz, 1H), 3.57 (d, J=13.6 Hz, 1H), 3.23-3.18 (m, 1H), 2.94-2.90 (m, 1H), 2.68-2.49 (m, 5H), 2.39-2.34 (m, 1H), 1.97-1.91 (m, 1H), 1.85-1.80 (m, 1H). LCMS: (Method C) 476.0 (M+H), Rt. 1.67 min, 99.27% (Max). HPLC: (Method A) Rt. 4.25 min, 99.53% (Max).

### 109

### Step 1: Methyl 2-((3S,5S)-1-(4-methoxybenzyl)-5-(4-(trifluoromethyl)phenyl)piperidin-3-yl)acetate

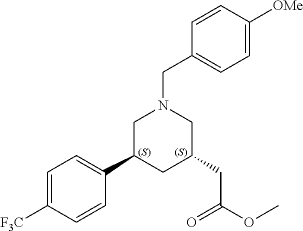

The title compound was synthesized by using methyl 2-((3S,5S)-5-(4-(trifluoromethyl)phenyl)piperidin-3-yl)acetate (100 mg, 0.33 mmol, Step-1 of Compound 101) and 1-(chloromethyl)-4-methoxybenzene (62.2 mg, 0.39 mmol) as described in step 4 for alkylation. Yield: 60% (85 mg, gummy solid). ¹H NMR (300 MHz, DMSO-d₆): δ 7.63 (d, J=8.4 Hz, 2H), 7.55 (d, J=7.8 Hz, 2H), 7.19 (d, J=8.7 Hz, 2H), 6.87 (d, J=8.7 Hz, 2H), 3.75 (s, 3H), 3.51 (s, 3H), 3.49-3.31 (m, 2H), 3.10-3.02 (m, 2H), 2.72-2.16 (m, 6H), 1.76-1.61 (m, 2H). LCMS: (Method C) 422.1 (M+H), Rt. 1.64 min, 99.31% (Max).

### Step 2: 2-((3S,5S)-5-(4-methoxybenzyl)-5-(4-(trifluoromethyl)phenyl)piperidin-3-yl)acetic acid

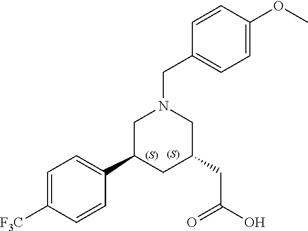

The title compound was synthesized by using methyl 2-((3S,5S)-1-(4-methoxybenzyl)-5-(4-(trifluoromethyl)phenyl)piperidin-3-yl)acetate (85 mg, 0.20 mmol) as described in step 5 for ester hydrolysis. Yield: 73% (25 mg, off white solid). ¹H NMR (400 MHz, CD₃OD): δ 7.65 (d, J=8.0 Hz, 2H), 7.51 (d, J=8.0 Hz, 2H), 7.40 (d, J=8.8 Hz, 2H), 6.99 (d, J=8.8 Hz, 2H), 4.12-4.02 (m, 2H), 3.82 (s, 3H), 3.37-3.32 (m, 3H), 3.05-2.97 (m, 2H), 2.61-2.50 (m, 3H), 2.10-2.05 (m, 1H), 1.95-1.91 (m, 1H). LCMS: (Method A) 408.2 (M+H), Rt. 2.03 min, 99.84% (Max), HPLC: (Method A) Rt. 3.29 min, 99.56% (Max).

### 110

### Step 1: Methyl 2-((3S,5S)-1-(3-methoxybenzyl)-5-(4-(trifluoromethyl)phenyl)piperidin-3-yl)acetate

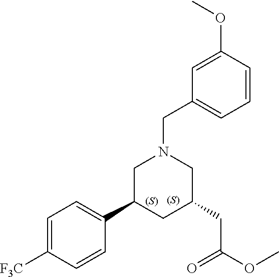

The title compound was synthesized by using methyl 2-((3S,5S)-5-(4-(trifluoromethyl)phenyl)piperidin-3-yl)acetate (100 mg, 0.33 mmol, Step-1 of Compound 101) and 1-(bromomethyl)-3-methoxybenzene (80.1 mg, 0.39 mmol) as described in step 4 for alkylation. Yield: 89% (125 mg, gummy solid). ¹H NMR (300 MHz, DMSO-d₆): δ 7.65-7.55 (m, 4H), 7.24-7.19 (m, 1H), 6.87-6.78 (m, 3H), 3.73 (s, 3H), 3.61-3.50 (s, 4H), 3.36-3.05 (m, 3H), 2.75-2.06 (m, 6H), 1.77-1.67 (m, 2H). LCMS: (Method C) 422.1 (M+H), Rt. 1.48 min, 97.40% (Max).

### Step 2: 2-((3S,5S)-1-(3-methoxybenzyl)-5-(4-(trifluoromethyl)phenyl)piperidin-3-yl)acetic acid

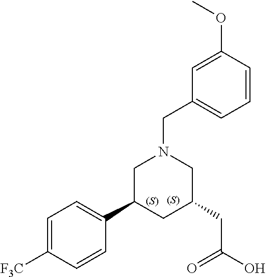

The title compound was synthesized by using methyl 2-((3S,5S)-1-(3-methoxybenzyl)-5-(4-(trifluoromethyl)phenyl)piperidin-3-yl)acetate (120 mg, 0.28 mmol) as described in step 5 for ester hydrolysis. Yield: 58% (68 mg, Off white solid). ¹H NMR (400 MHz, CD₃OD): δ 7.62 (d, J=8.4 Hz, 2H), 7.51 (d, J=8.4 Hz, 2H), 7.32-7.28 (m, 1H), 7.03-6.98 (m, 2H), 6.93-6.91 (m, 1H), 3.95-3.83 (m, 2H), 3.82 (s, 3H), 3.33-3.29 (m, 1H), 3.15-3.02 (m, 2H), 2.85-2.74 (m, 2H), 2.64-2.58 (m, 2H), 2.45-2.38 (m, 1H), 2.03-1.88 (m, 2H). LCMS: (Method A) 408.0 (M+H), Rt. 2.32 min, 99.17% (Max). HPLC: (Method A) Rt. 3.80 min, 99.80% (Max).

### 111

### Step 1: Methyl 2-((3S,5S)-1-(3-(trifluoromethyl)benzyl)-5-(4-(trifluoromethyl)phenyl)piperidin-3-yl)acetate

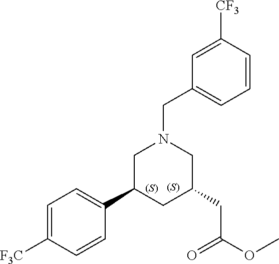

The title compound was synthesized by using methyl 2-((3S,5S)-5-(4-(trifluoromethyl)phenyl)piperidin-3-yl)acetate (100 mg, 0.33 mmol, Step-1 of Compound 101) and 1-(bromomethyl)-3-(trifluoromethyl)benzene (95.29 mg, 0.39 mmol) as described in step 4 for alkylation. Yield: 59% (90 mg, Gummy solid). LCMS: (Method C) 460.0 (M+H), Rt. 2.06 min, 19.94% (Max).

### Step 2: 2-((3S,5S)-1-(3-(trifluoromethyl)benzyl)-5-(4-(trifluoromethyl)phenyl)piperidin-3-yl)acetic acid

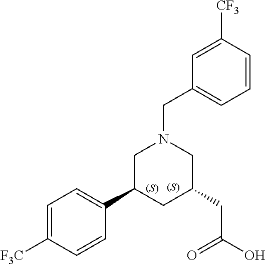

The title compound was synthesized by using methyl 2-((3S,5S)-1-(3-(trifluoromethyl)benzyl)-5-(4-(trifluoromethyl)phenyl)piperidin-3-yl)acetate (90 mg, 0.196 mmol) as described in step 5 for ester hydrolysis. Yield: 42% (35 mg, Off white solid). ¹H NMR (400 MHz, CD₃OD): δ 7.70 (s, 1H), 7.65 (d, J=7.2 Hz, 1H), 7.60-7.51 (m, 6H), 3.82-3.79 (m, 1H), 3.68-3.64 (m, 1H), 3.23-3.17 (m, 1H), 2.97-2.92 (m, 1H), 2.75-2.50 (m, 5H), 2.40-2.36 (m, 1H), 1.97-1.91 (m, 1H), 1.85-1.80 (m, 1H). LCMS: (Method D) 446.1 (M+H), Rt. 2.27 min, 99.68% (Max). HPLC: (Method A) Rt. 4.15 min, 99.94% (Max).

### 112

### Step 1: 1-(chloromethyl)-4-ethynylbenzene

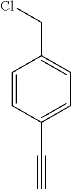

To a stirred solution of (4-ethynylphenyl)methanol (0.2 g, 1.51 mmol) in DCM (10 mL) was added SOCl₂ (1.87 mL, 25.7 mmol) at 0° C. After stirring for 5 min, the reaction mixture was stirred at 50° C. for 4 h. The reaction mixture was monitored by TLC. After completion, the reaction mixture was concentrated. The resulting residue was dissolved in DCM (15 mL), washed with 10% aq. NaHCO₃ solution, water (10 mL) and brine (10 mL). The organic layer was dried over anhydrous sodium sulphate and the solvent was evaporated under vacuum to get the title compound which was used directly in the next step. Yield: 83% (190 mg, gummy solid).

### Step 2: Methyl 2-((3S,5S)-1-(4-ethynylbenzyl)-5-(4-(trifluoromethyl)phenyl)piperidin-3-yl)acetate

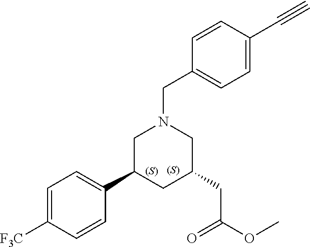

The title compound was synthesized by using methyl 2-((3S,5S)-5-(4-(trifluoromethyl)phenyl)piperidin-3-yl)acetate (0.2 g, 0.66 mmol, Step-1 of Compound 101) and 1-(chloromethyl)-4-ethynylbenzene (130 mg, 0.86 mmol) as described in step 4 for alkylation. Yield: 33% (90 mg, gummy solid). LCMS: (Method A) 416.0 (M+H), Rt. 2.47 min, 55.73% (Max).

### Step 3: 2-((3S,5S)-1-(4-ethynylbenzyl)-5-(4-(trifluoromethyl)phenyl)piperidin-3-yl)acetic acid

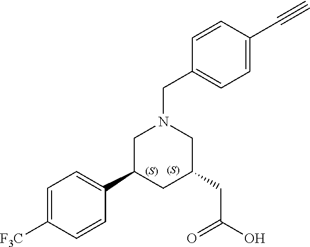

The title compound was synthesized by using methyl 2-((3S,5S)-1-(4-ethynylbenzyl)-5-(4-(trifluoromethyl)phenyl)piperidin-3-yl)acetate (90 mg, 0.216 mmol) as described in step 5 for ester hydrolysis. Yield: 40% (35 mg, Off white solid). ¹H NMR (400 MHz, CD₃OD): δ 7.60 (d, J=8.0 Hz, 2H), 7.52 (d, J=8.4 Hz, 2H), 7.46 (d, J=8.0 Hz, 2H), 7.38 (d, J=8.0 Hz, 2H), 3.76-3.73 (m, 1H), 3.68-3.64 (m, 1H), 3.49 (s, 1H), 3.27-3.20 (m, 1H), 2.97-2.94 (m, 1H), 2.80-2.73 (m, 1H), 2.63-2.50 (m, 4H), 2.40-2.35 (m, 1H), 1.97-1.96 (m, 1H), 1.97-1.82 (m, 1H). LCMS: (Method D) 402.1 (M+H), Rt. 2.16 min, 99.36% (Max). HPLC: (Method A) Rt. 3.88 min, 99.70% (Max).

### 113 & 114 Stereoisomers

### Step 1: Methyl 2-((3S,5S)-1-(4-(trifluoromethyl)benzyl)-5-(4-(trifluoromethyl)phenyl)piperidin-3-yl)acetate

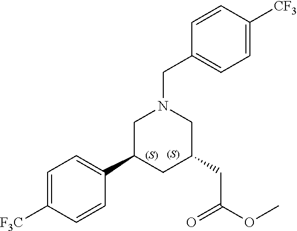

The title compound was synthesized by using methyl 2-((3S,5S)-5-(4-(trifluoromethyl)phenyl)piperidin-3-yl)acetate (0.2 g, 0.66 mmol, Step-1 of Compound 101) and 1-(bromomethyl)-4-(trifluoromethyl)benzene (206.4 mg, 0.86 mmol) as described in step 4 for alkylation. Yield: 72% (220 mg, gummy solid). ¹H NMR (400 MHz, CDCl₃): δ 7.59-7.56 (m, 4H), 7.47-7.38 (m, 4H), 3.64 (s, 3H), 3.53-3.49 (m, 2H), 3.12-3.10 (m, 1H), 2.88-2.86 (m, 1H), 2.76-2.72 (m, 1H), 2.58-2.52 (m, 2H), 2.39-2.31 (m, 3H), 1.82-1.79 (m, 2H). LCMS: (Method B) 460.1 (M+H), Rt. 2.87 min, 99.32% (Max).

### Step 2: Methyl 2-((3S,5S)-1-(4-(trifluoromethyl)benzyl)-5-(4-(trifluoromethyl)phenyl)piperidin-3-yl)propanoate

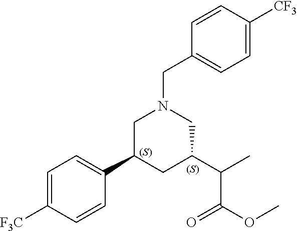

A solution of LDA (0.28 mL, 0.55 mmol, 2 M in THF) was added over 5 min to a stirred suspension of methyl 2-((3S,5S)-1-(4-(trifluoromethyl)benzyl)-5-(4-(trifluoromethyl)phenyl)piperidin-3-yl)acetate (210 mg, 0.46 mmol) in THF at −78° C. The reaction mixture was stirred at −78° C. for 1 h. Then methyl iodide (130 mg, 0.91 mmol) added and the reaction mixture was slowly warmed to RT overnight. After completion (the reaction mixture was monitored by LCMS), the reaction mixture was quenched by dropwise addition of sat. aq. NH₄Cl solution. The reaction mixture was stirred for 10 min at RT and then extracted with EtOAc (3×20 mL). The combined organic layer was washed with water (200 mL), brine (200 mL), dried over anhydrous Na₂SO₄ and concentrated under vacuum to get the title compound which was used in the next step without further purification. Yield: 65% (140 mg, gummy solid). LCMS: (Method B) 474.0 (M+H), Rt. 2.56 min, 95.37% (Max).

### Step 3: (R)-2-((3S,5S)-1-(4-(trifluoromethyl)benzyl)-5-(4-(trifluoromethyl)phenyl)piperidin-3-yl)propanoic acid& (S)-2-((3S,5S)-1-(4-(trifluoromethyl)benzyl)-5-(4-(trifluoromethyl)phenyl)piperidin-3-yl)propanoic acid

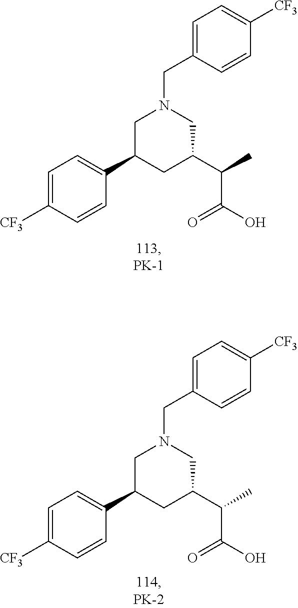 [SMILES: C[C@@H](C(=O)O)[C@@H]1C[C@@H](c2ccc(C(F)(F)F)cc2)CN(Cc2ccc(C(F)(F)F)cc2)C1 ; C[C@H](C(=O)O)[C@@H]1C[C@@H](c2ccc(C(F)(F)F)cc2)CN(Cc2ccc(C(F)(F)F)cc2)C1]

The title compound was synthesized by using methyl 2-((3S,5S)-1-(4-(trifluoromethyl)benzyl)-5-(4-(trifluoromethyl)phenyl)piperidin-3-yl)propanoate (140 mg, 0.295 mmol) as described in step 5 for ester hydrolysis. Yield: 44% (60 mg, Off white solid). The mixture of two diastereomers thus obtained was separated by chiral SFC purification (Method C). The structures are assigned arbitrarily.

113 (Stereoisomer1): Yield: 9% (12.51 mg, Off white solid). ¹H NMR (400 MHz, CD₃OD): δ 7.67 (d, J=8.0 Hz, 2H), 7.61-7.58 (m, 4H), 7.51 (d, J=8.4 Hz, 2H), 3.64-3.50 (m, 2H), 3.33-3.32 (m, 1H), 3.00-2.98 (m, 1H), 2.85-2.83 (m, 2H), 2.57-2.51 (m, 2H), 1.99-1.84 (m, 3H), 1.05 (d, J=6.8 Hz, 3H). LCMS: (Method A) 460.0 (M+H), Rt. 2.46 min, 98.45% (Max), HPLC: (Method A) Rt. 4.25 min, 99.95% (Max).

114 (Stereoisomer2): Yield: 7% (9.52 mg, Off white solid). ¹H NMR (400 MHz, CD₃OD): δ 7.63-7.56 (m, 8H), 3.64-3.51 (m, 2H), 3.16-3.13 (m, 1H), 2.75-2.52 (m, 5H), 2.04-1.78 (m, 3H), 1.12 (d, J=7.2 Hz, 3H). LCMS: (Method A) 459.9 (M+H), Rt. 2.51 min, 96.48% (Max), HPLC: (Method A) Rt. 4.30 min, 95.69% (Max).

### 115

### Step 1: Methyl 2-((3S,5S)-1-(4-cyanobenzyl)-5-(4-(trifluoromethyl)phenyl)piperidin-3-yl)acetate

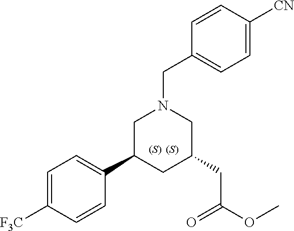

The title compound was synthesized by using methyl 2-((3S,5S)-5-(4-(trifluoromethyl)phenyl)piperidin-3-yl)acetate (150 mg, 0.49 mmol, Step 1 of Compound 101) and 4-(bromomethyl)benzonitrile (117.20 mg, 0.59 mmol) as described in step 4 for alkylation. Yield: 72% (150 mg, gummy solid). ¹H NMR (400 MHz, CDCl₃): δ 7.64-7.54 (m, 4H), 7.51-7.38 (m, 4H), 3.66 (s, 3H), 3.64-3.51 (m, 2H), 3.13-3.08 (m, 1H), 2.88-2.26 (m, 7H), 1.85-1.78 (m, 2H). LCMS: (Method A) 417.0 (M+H), Rt. 2.38 min, 99.26% (Max).

### Step 2: 2-((3S,5S)-1-(4-cyanobenzyl)-5-(4-(trifluoromethyl)phenyl)piperidin-3-yl)acetic acid

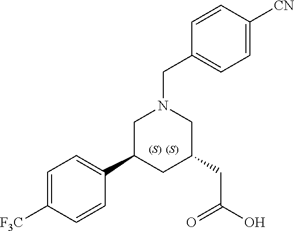

The title compound was synthesized by using methyl 2-((3S,5S)-1-(4-cyanobenzyl)-5-(4-(trifluoromethyl)phenyl)piperidin-3-yl)acetate (150 mg, 0.36 mmol) as described in step 5 for ester hydrolysis. Yield: 48% (70 mg, Off white solid). ¹H NMR (400 MHz, DMSO-d₆): δ 12.03 (bs, 1H), 7.79 (d, J=8.0 Hz, 2H), 7.65 (d, J=8.4 Hz, 2H), 7.58-752 (m, 4H), 3.64-3.60 (m, 1H), 3.56-3.52 (m, 1H), 3.14-3.06 (m, 1H), 2.76-2.31 (m, 6H), 2.21-1.13 (m, 1H), 1.78-1.71 (m, 1H), 1.68-1.63 (m, 1H). LCMS: (Method A) 402.9 (M+H), Rt. 2.26 min, 98.85% (Max). HPLC: (Method A) Rt. 3.53 min, 99.18% (Max).

In some cases, the compounds are synthesized with the general methods below.

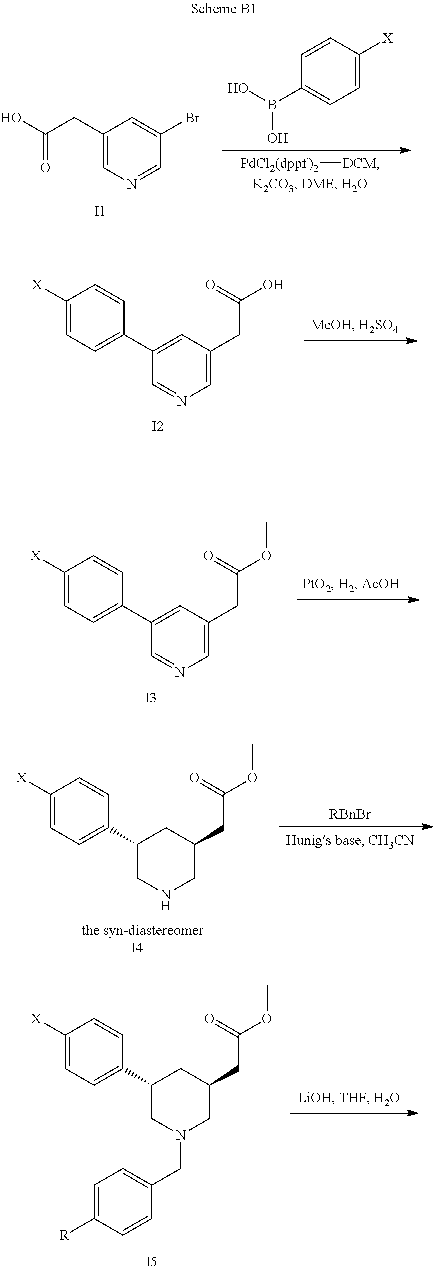

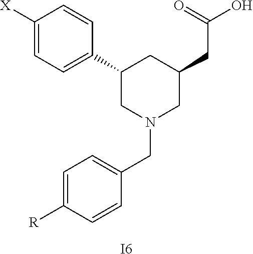

In general, starting with meta bromopyridine I1 the biaromatic compound can be synthesized using standard Suzuki conditions such Palladium chloride DPPF, base and the appropriately substituted aryl or heteroaryl boronic acid to form functionalized pyridine I2. This pyridine can then be esterified under standard conditions and the resulting ester can be reduced using hydrogen gas and a metal catalyst such as Platinum oxide. The resulting piperidine I4 is formed as a mixture of isomers. The S,S trans isomer can be separated by chromatographic or crystallographic methods to produce isomer I4. Alternately the cis and trans isomers can be separated and the additional chemistry can be conducted on the racemate. The trans piperidine I4 can then be alkylated using a substituted aryl or heteroaryl benzyl bromide. Alternately the alkylation can occur using an aromatic aldehyde and reductive amination (i.e., NaBH₄), or using a primary alcohol and mitsunobu conditions. The ester can then be hydrolyzed using standard conditions such as aqueous LiOH to form 16. If the X substituent is sensitive to hydrogenation or acidic conditions (ie X═CN, CCH) it can be made using route outlined in Scheme B2.

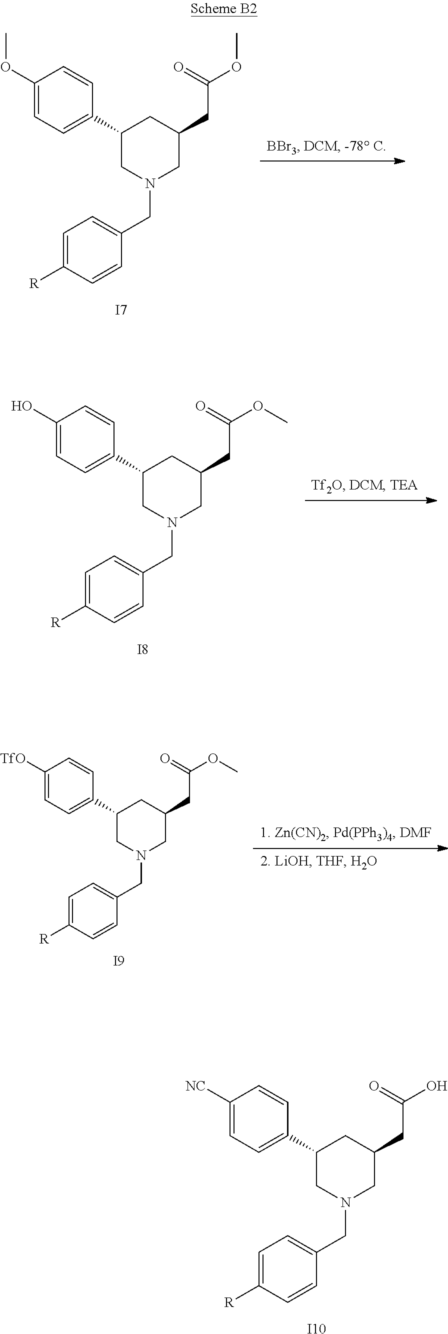

In these cases, the methoxyl substituted phenyl prepared by Scheme 100 above is dealkylated using standard Lewis acid conditions such as BBr₃. The resulting phenol I8 is converted to the triflate using triflic anhydride and a base. This triflate can then be coupled with desired substituent (i.e., ZnCN) using a palladium catalyst. The resulting ester can then be hydrolyzed under basic conditions to form the desired product I10. The methylene alpha to the carboxylic acid can be further functionalized using the conditions outlined in Scheme B3. Starting with the ester I5, an enolate can be alkylated using an alkyl halide such as methyl iodide and the resulting ester can be hydrolyzed to the acid I11 using standard basic conditions.

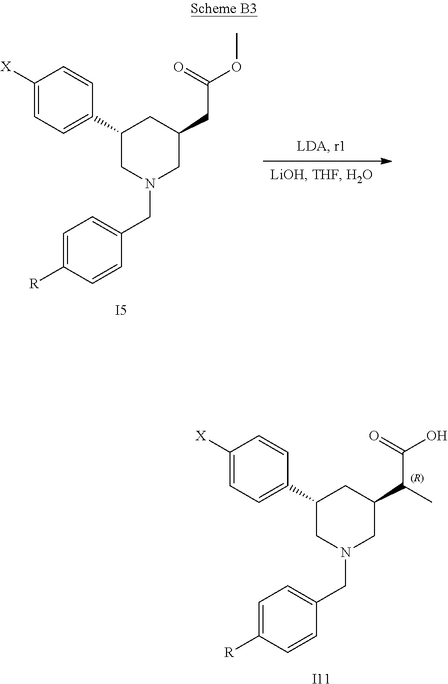

### Synthesis of Compounds 116-146

### General Information:

### LCMS Analysis Condition:

Instrument name: Agilent Technologies 1290 infinity 11.

Method A: Method: A—0.1% TFA in H₂O, B—0.1% TFA in ACN; flow rate: 2.0 mL/min; column: XBridge C8 (50×4.6 mm, 3.5 μm) or BEH C8, +ve mode.

Method B: Method: A—10 mM NH₄HCO₃ in H₂O, B—ACN; flow rate: 1.0 mL/min; column: XBridge C8 (50×4.6 mm, 3.5 μm) or BEH C8, +ve mode.

Method C: Method: A—0.1% HCOOH in H₂O, B—0.1% FA in ACN; flow rate: 1.5 mL/min; column: ZORBAX XDB C-18 (50×4.6 mm, 3.5 μm) or BEH C8, +ve mode.

Method D: Method: A—10 mM NH₄OAc in H₂O, B—ACN; flow rate: 1.0 mL/min; column: XBridge C8 (50×4.6 mm, 3.5 μm) or BEH C8, +ve mode.

Instrument name: Agilent 1260 infinity II.

Method E: Method: A—0.1% NH₃H₂O in H₂O, B—ACN; flow rate: 2.0 mL/min; column: XBridge C18 (50×4.6 mm, 3.5 μm), ESI mode.

Method F: Method: A—0.05% FA in H₂O, B—0.05% FA in ACN; flow rate: 2.0 mL/min: column: Welch Boltimate EXT C18 core-shell (4.6*50 mm, 2.7 μm). ESI mode.

Instrument name: Agilent 1260-6120B QuaMS.

Method G: Method: A—0.05% TFA in H₂O, B—ACN; flow rate: 2.0 mL/min; Column: Welch Boltimate C18 core-shell (4.6*50 mm, 2.7 μm). ESI mode.

Instrument name: waters UPLC H-Class.

Method H: Method: A—0.05% FA in H₂O, B—0.05% FA in ACN; flow rate: 0.6 mL/min; column: Waters UPLC BEH C18 (2.1 mm*50 mm 1.7 μm). ESI mode.

### HPLC Analysis Condition:

Instrument name: Agilent 1200 Series instruments as followed using % with UV detection (maxplot).

Method A: Method: A—0.1% TFA in H₂O, B—0.1% TFA in ACN; flow rate: 2.0 mL/min; column: XBridge C8 (50×4.6 mm, 3.5 μm).

Method B: Method: A—10 mM NH₄HCO₃ in H₂O, B-ACN; flow rate: 1.0 mL/min; column: XBridge C8 (50×4.6 mm, 3.5 μm).

Instrument name: Waters UPLC as followed using % with UV detection (maxplot).

Method C: Method: A—0.02% TFA in H₂O, B—0.02% TFA in ACN; flow rate: 2.0 mL/min; column: ACQUITY UPLC BEH C18 (2.1*150 mm, 1.7 μm).

Method D: Method: A—10 mM NH₄HCO₃ in H₂O, B-ACN; flow rate: 1.0 mL/min; column: YMC Triart C18 (4.6*150 mm, 3 μm).

Method E: Method: A—0.02% TFA in H₂O, B—0.02% TFA in ACN; flow rate: 0.4 mL/min; column: Welch Ultimate UHPLC LP-C18 (2.1*100 mm, 1.8 μm).

### Prep-HPLC Purification Condition:

Method A: A—0.1% TFA in H₂O, B-MeOH or ACN; column: Sunfire C8 (19×250 mm, 5 μm) or Sunfire C18 (30×250 mm, 10 μm).

Method B: A—10 mM NH₄HCO₃ in H₂O, B-MeOH or ACN, Column: Sunfire C8 (19×250 mm, 5 μm) or Sunfire C18 (30×250 mm, 10 μm).

Method C: A—0.1% FA in H₂O, B-ACN; column: Welch Ultimate XB-C18 (21.2*150 mm 5 um) or (50*150 mm, 5 μm).

Method D: A—0.1% NH₃H₂O/H₂O, B-ACN, column: Xbridge C18 (19*250 mm 5 μm).

Method E: A—0.05% TFA in H₂O, B-CAN, column: Waters SunFire C18 OBD (19*150 mm 5 μm).

### Chiral SFC Purification Condition:

Instrument name: PIC-P10-20 (analytical).

Method A: Mobile Phase: 0.1% Isopropylamine in IPA:MeOH (1:1), flow rate: 3 mL/min; column: Lux A1 (250×4.6 mm 5 μm).

Method B: Mobile Phase: 0.1% Isopropylamine in IPA:MeOH (1:1), flow rate: 3 mL/min; column: Chiralpak OX-H (250×4.6 mm 5 μm).

Method C: Mobile Phase: 0.5% Isopropylamine in MeOH (1:1), flow rate: 3 mL/min; column: Lux C3 (250×4.6 mm 5 μm).

Instrument name: Waters Acquity UPCC.

Method D: Mobile phase: CO₂/EtOH [1% NH3 (7M in MeOH)]=85/15 Flow rate: 3 mL/min; Column: Daicel OJ-3 (4.6*100 mm 3 μm).

Method E: Mobile phase: CO₂/MeOH [0.2% NH3 (7M in MeOH)]=65/35, flow rate: 3 mL/min; Column: YMC Cellulose-SC (4.6*100 mm 3 μm).

Method F: Mobile phase: CO₂/MeOH [0.2% NH₃ (7M in MeOH)]=85/15; Flow rate: 3 mL/min: OJ-3 (4.6*100 mm 3 μm).

Method G: Mobile phase: CO₂/MeOH [0.2% NH₃ (7M in MeOH)]=65/35; Flow rate: 3 mL/min; YMC Cellulose-SC (4.6*100 mm 3 μm).

Method H: Mobile Phase: CO₂/MeOH (0.1% DEA)=80:20, flow rate: 2.5 mL/min; column: OJ-H (4.6*150 mm 3 μm).

Method I: Mobile Phase: CO₂:MeOH (0.05% DEA)=80:20, flow rate: 2.5 mL/min; column: AD-H (4.6*50 mm 3 μm).

### Chiral SFC Purification Condition:

Instrument name: PIC SFC 175

Method A: Mobile Phase: 0.1% Isopropylamine in IPA:MeOH (1:1), flow rate: 100 mL/min; column: Lux A1 (250×30 mm, 5 μm).

Method B: Mobile Phase: 0.1% Isopropylamine in IPA:MeOH (1:1), flow rate: 100 mL/min; column: Chiralpak OX-H (250×30 mm, 5 μm).

Method C: Mobile Phase: 0.5% Isopropylamine in IPA:MeOH (1:1), flow rate: 100 mL/min; column: Lux A1 (250×30 mm, 5 μm).

Instrument name: SFC-150mgm (Waters).

Method D: Mobile phase: CO₂/EtOH [0.5% NH3 (7M in MeOH)]=90/10, Flow rate: 100 mL/min; Column: Daicel OJ (25*250 mm, 10 μm).

Method E: Mobile phase: CO₂/MeOH [0.2% NH₃ (7M in MeOH)]=65/35, Flow rate: 100 mL/min; column: YMC Cellulose-SC (25*250 mm, 5 μm).

Method F: Mobile phase: CO₂/MeOH [0.2% NH₃ (7M in MeOH)]=90/10; Flow rate: 120 mL/min; Daicel OJ-3 (25*250 mm, 10 μm).

Method G: Mobile phase: CO₂/MeOH [0.2% NH₃ (7M in MeOH)]=70/30, Flow rate: 100 mL/min; column: YMC Cellulose-SC (20*250 mm, 5 μm).

Instrument name: Waters 80Q

Method H: Mobile Phase: CO₂/MeOH (0.1% NH₃H₂O)=80:20, flow rate: 60 mL/min; column: OJ-H (30*250 mm, 5 μm).

Method I: Mobile Phase: CO₂/MeOH (0.1% NH₃H₂O)=80:20, flow rate: 50 mL/min; column: AD-H (30*250 mm, 5 μm).

NMR instrument name: BRUKER NMR, model AV-II, AV-III and AV-NEO 400 MHz FT-NMR.

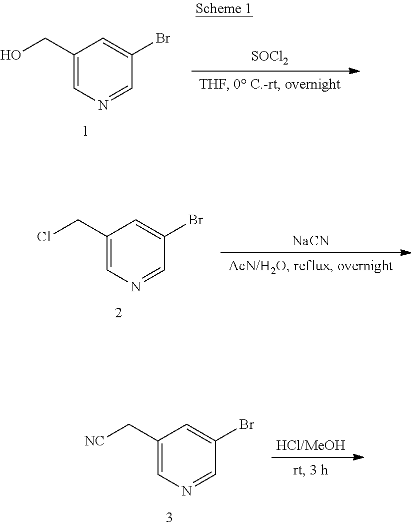

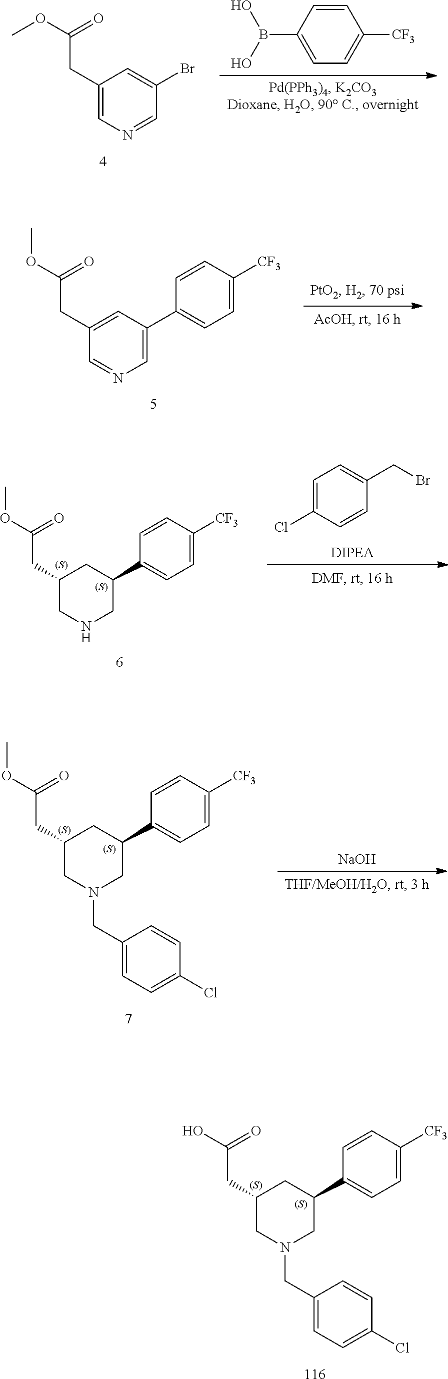

### Step 1: 3-bromo-5-(chloromethyl)pyridine

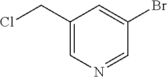

To a solution of (5-bromopyridin-3-yl)methanol (50.0 g, 267 mmol) in tetrahydrofuran (100 mL) thionylchloride (75.0 mL, 1.03 mol) was added dropwise while cooling in an ice-bath. The reaction mixture was warmed and stirred at room temperature overnight. Then the mixture was poured into ice water (200 mL), basified with 10 N aq. NaOH (80 mL) and extracted with ethyl acetate (150 mL×3). The combined organic layer was dried over anhydrous sodium sulfate, filtered, and concentrated to give the title compound. Yield: crude (57.0 g, brown solid). LCMS: (Method H) 206.1 (M+H).

### Step 2: 2-(5-bromopyridin-3-yl)acetonitrile

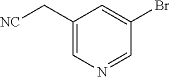

To a solution of 3-bromo-5-(chloromethyl)pyridine (crude, 48.0 g, 234 mmol) in ACN (500 mL) was added sodium cyanide (17.2 g, 351 mmol) dissolved in water (90 mL). The mixture was refluxed overnight. After cooled to room temperature, the mixture was poured into water (1000 mL), and extracted with dichloromethane (500 mL×3). The combined organic layer was washed by brine, dried over anhydrous sodium sulfate, filtered and concentrated. The residue was purified by silica gel column chromatography (tert-butyl methyl ether/petroleum ether=3/7) to give the title compound. Yield: 63% over two steps (27.9 g, yellow solid). LCMS: (Method H) 197.1 (M+H).

### Step 3: methyl 2-(5-bromopyridin-3-yl)acetate

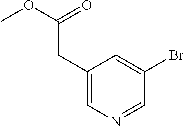

2-(5-Bromopyridin-3-yl)acetonitrile (27.9 g, 142 mmol) was dissolved in a solution of 2 M hydrochloric acid in methanol (300 mL). The reaction was stirred at room temperature for 3 hours. When the reaction was completed, the solvent was removed. The mixture was diluted with saturated sodium bicarbonate solution (100 mL) and extracted with ethyl acetate (300 mL×2). The organic layer was washed with brine, dried over anhydrous sodium sulfate, filtered, and concentrated. The residue was purified by silica gel column chromatography (tert-butyl methyl ether/petroleum ether=3/7) to give the title compound. Yield: 64% (20.9 g, brown oil). LCMS: (Method H) 230.1 (M+H).

### Step 4: methyl 2-(5-(4-(trifluoromethyl)phenyl)pyridin-3-yl)acetate

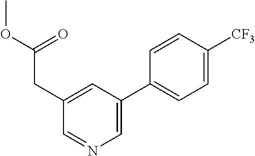 [SMILES: COC(=O)Cc1cncc(-c2ccc(C(F)(F)F)cc2)c1]

To a solution of methyl 2-(5-bromopyridin-3-yl)acetate (20.9 g, 91.2 mmol), (4-(trifluoromethyl)phenyl)boronic acid (20.8 g, 110 mmol) and potassium carbonate (37.8 g, 274 mmol) in 1,4-dioxane (450 mL) and water (50 mL) was added tetrakis(triphenylphosphine)palladium (1.30 g, 1.14 mmol). The mixture was degassed and flushed with N₂ three times. The reaction was stirred at 90° C. under nitrogen atmosphere overnight. After the mixture was cooled to room temperature, the mixture was diluted with ethyl acetate and washed with brine. The organic layer was dried over anhydrous sodium sulfate, filtered, and concentrated. The residue was purified by silica gel column chromatography (tert-butyl methyl ether/petroleum ether=7/3) to give the title compound. Yield: 82% (22.3 g, orange solid). LCMS: (Method H) 296.1 (M+H).

### Step 5: methyl 2-((3S,5S)-5-(4-(trifluoromethyl)phenyl)piperidin-3-yl)acetate

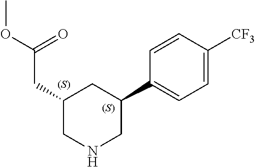

To a stirred solution of methyl 2-(5-(4-(trifluoromethyl)phenyl)pyridin-3-yl)acetate (22.3 g, 75.5 mmol) in acetic acid (250 mL) was added platinum dioxide (4.80 g, 21.1 mmol). The reaction mixture was stirred under hydrogen atmosphere (70 psi) for 16 h at room temperature. When the reaction was completed, the reaction mixture was filtered. The filtrate was concentrated, and the residue was neutralized with saturated sodium bicarbonate solution. The resulting suspension was extracted with ethyl acetate (500 mL×2). The combined organic layer was washed with brine, dried over anhydrous sodium sulfate, filtered, and concentrated. The residue was purified by silica gel column chromatography (dichloromethane:methanol=30:1) to give anti-methyl 2-(5-(4-(trifluoromethyl)phenyl)piperidin-3-yl)acetate (4.70 g, as brown oil). The two isomers were separated by Chiral SFC to give the title compound as fraction 1. Chiral SFC: (Method I) Rt. 0.760 min, 46.44% (Max) and Rt. 1.118 min, 53.56% (Max). Yield: 7% (1.7 g, white solid). LCMS: (Method H) 302.1 (M+H). ¹H NMR (400 MHz, DMSO-d₆): δ 7.64 (d, J=8.0 Hz, 2H), 7.52 (d, J=8.0 Hz, 2H), 3.59 (s, 3H), 2.97-2.87 (m, 2H), 2.76 (dd, J=12.2, 3.4 Hz, 1H), 2.68-2.61 (m, 3H), 2.50-2.47 (m, 1H), 2.13-2.08 (m, 1H), 1.88-1.81 (m, 1H), 1.71-1.68 (m, 1H). Chiral SFC: (Method 1) Rt. 0.806 min, 100/6 (Max).

### Step 6: methyl 2-((3S,5S)-1-(4-chlorobenzyl)-5-(4-(trifluoromethyl)phenyl)piperidin-3-yl)acetate

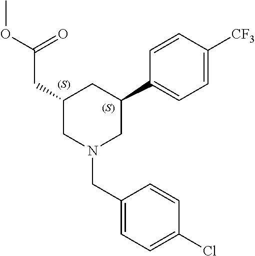

To a stirred solution of methyl 2-((3S,5S)-5-(4-(trifluoromethyl)phenyl)piperidin-3-yl)acetate (50.0 mg, 0.166 mmol) and N,N-diisopropylethylamine (74.0 mg, 0.573 mmol) in N,N-dimethylformamide (1 mL) was added 1-(bromomethyl)-4-chlorobenzene (41.0 mg, 0.199 mmol). The mixture was stirred at room temperature for 16 h. The reaction mixture was diluted with water (5 mL) and extracted with ethyl acetate (3 mL×3). The combined organic layer was washed with brine, dried over anhydrous sodium sulfate, filtered, and concentrated. The residue was purified by silica gel column chromatography (ethyl acetate/petroleum ether=1/10) to give the title compound. Yield: 94% (67.0 mg, brown oil). LC-MS: (Method H) 426.1 (M+H).

### Step 7: 2-((3S,5S)-1-(4-chlorobenzyl)-5-(4-(trifluoromethyl)phenyl)piperidin-3-yl)acetic acid 2-((3S,5S)-1-(4-chlorobenzyl)-5-(4-(trifluoromethyl)phenyl)piperidin-3-yl)acetic acid (116)

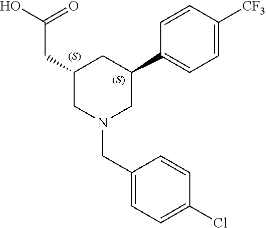

To a solution of methyl 2-((3S,5S)-1-(4-chlorobenzyl)-5-(4-(trifluoromethyl)phenyl)piperidin-3-yl)acetate (67.0 mg, 0.157 mmol) in tetrahydrofuran (0.5 mL) and methanol (0.5 mL) was added sodium hydroxide (16.0 mg, 0.394 mmol) in water (1.0 mL). The mixture was stirred at room temperature for 3 h. The organic solvent was removed. The residue was acidified with 3 N HCl to pH 4˜5. The precipitated solid was filtered, and purified by Prep-HPLC to give the title compound. Prep-HPLC (method C). Yield: 43% (28 mg, off-white solid). ¹H NMR (400 MHz, CDCl₃) δ 7.54 (d, J=8.0 Hz, 2H), 7.33 (d, J=8.0 Hz, 2H), 7.30 (s, 4H), 3.71 (s, 2H), 3.30-3.24 (m, 1H), 3.09-2.97 (m, 2H), 2.95-2.85 (m, 1H), 2.68-2.58 (m, 1H), 2.57-2.49 (m, 1H), 2.48-2.40 (m, 1H), 2.31-2.28 (m, 1H), 1.91-1.83 (m, 2H). LC-MS: (Method H) 412.2 (M+H), Rt. 1.77 min, 100.00% (Max). HPLC: (Method E) Rt. 3.60 min, 98.88% (Max).

### 117

### 2-((3S,5S)-1-(4-fluorobenzyl)-5-(4-(trifluoromethyl)phenyl)piperidin-3-yl)acetic acid

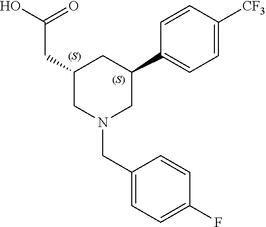

117 was synthesized following the route of scheme 1, substituting 1-(bromomethyl)-4-fluorobenzene in step 6.

¹H NMR (400 MHz, CDCl₃) δ 7.53 (d, J=8.0 Hz, 2H), 7.34-7.32 (m, 4H), 7.01 (t, J=8.8 Hz, 2H), 3.73 (s, 2H), 3.30-3.24 (m, 1H), 3.07-3.05 (m, 2H), 2.91-2.85 (m, 1H), 2.64-2.44 (m, 2H), 2.34 (s, 1H), 2.32 (t, J=10.8 Hz, 1H), 1.90-1.82 (m, 2H). LCMS: (Method H) 396.1 (M+H), Rt. 1.73 min, 100.00% (Max). HPLC: (Method E) Rt. 3.42 min, 98.82% (Max).

### 118

### 2-((3S,5S)-1-(3,4-difluorobenzyl)-5-(4-(trifluoromethyl)phenyl)piperidin-3-yl)acetic acid

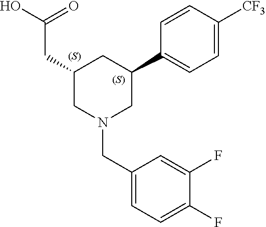

118 was synthesized following the route of scheme 1, substituting 4-(bromomethyl)-1,2-difluorobenzene in step 6.

¹H NMR (400 MHz, CDCl₃) δ 7.53 (d, J=8.0 Hz, 2H), 7.35 (d, J=8.0 Hz, 2H), 7.19-7.12 (m, 1H), 7.10-7.01 (m, 2H), 3.53 (s, 2H), 3.16-3.13 (m, 1H), 2.93-2.91 (m, 1H), 2.80-2.77 (m, 2H), 2.61-2.56 (m, 1H), 2.43-2.41 (m, 2H), 2.26 (t, J=9.6 Hz, 1H), 1.89-1.74 (m, 2H). LCMS: (Method H) 414.2 (M+H), Rt. 1.83 min, 100.00% (Max). HPLC: (Method E) Rt. 3.48 min, 100.00% (Max).

### 119

### 2-((3S,5S)-1-(2-fluorobenzyl)-5-(4-(trifluoromethyl)phenyl)piperidin-3-yl)acetic acid

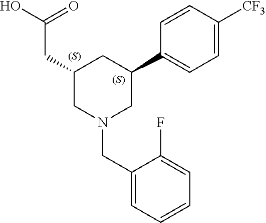

119 was synthesized following the route of scheme 1, substituting 1-(bromomethyl)-2-fluorobenzene in step 6.

¹H NMR (400 MHz, CDCl₃) δ 7.54 (d, J=8.0 Hz, 2H), 7.42 (t, J=8.0 Hz, 1H), 7.36 (d, J=8.0 Hz, 2H), 7.32-7.24 (m, 1H), 7.13 (t, J=8.0 Hz, 1H), 7.05 (t, J=8.0 Hz, 1H), 3.83-3.70 (m, 2H), 3.21 (m, 1H), 3.09-2.89 (m, 2H), 2.78 (m, 1H), 2.59 (m, 2H), 2.40 (m, 2H), 1.93-1.74 (m, 2H). LCMS: (Method H) 396.2 (M+H), Rt. 1.75 min, 100.00% (Max). HPLC: (Method E) Rt. 3.37 min, 97.55% (Max).

### 120

### 2-((3S,5S)-1-(4-(methylsulfonyl)benzyl)-5-(4-(trifluoromethyl)phenyl)piperidin-3-yl)acetic acid

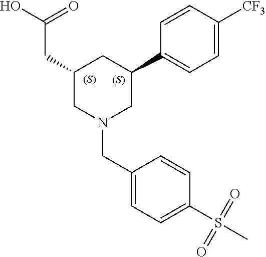

120 was synthesized following the route of scheme 1, substituting 1-(bromomethyl)-4-(methylsulfonyl)benzene in step 6.

¹H NMR (400 MHz, CD₃OD) δ 7.92 (d, J=8.4 Hz, 2H), 7.65 (d, J=8.4 Hz, 2H), 7.58 (d, J=8.4 Hz, 2H), 7.50 (d, J=8.0 Hz, 2H), 3.77 (q, J=13.6 Hz, 2H), 3.30-3.19 (m, 1H), 3.11 (s, 3H), 3.01-2.98 (m, 1H), 2.76-2.66 (m, 2H), 2.59-2.50 (m, 3H), 2.41-2.32 (m, 1H), 1.97-1.76 (m, 2H). LCMS: (Method H) 456.2 (M+H), Rt. 1.46 min, 100.00% (Max). HPLC: (Method E) Rt. 3.10 min, 99.55% (Max).

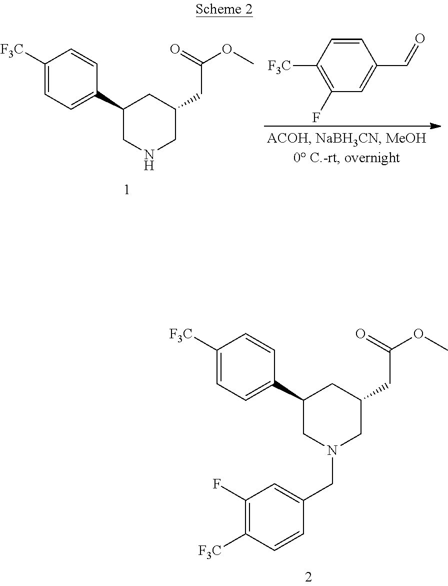

### Step 1: anti-methyl 2-(1-(3-fluoro-4-(trifluoromethyl)benzyl)-5-(4-(trifluoromethyl)phenyl)piperidin-3-yl)acetate

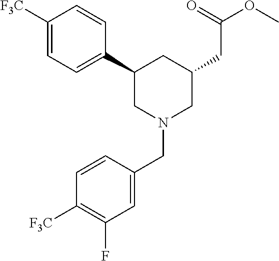 [SMILES: COC(=O)C[C@H]1C[C@H](c2ccc(C(F)(F)F)cc2)CN(Cc2ccc(C(F)(F)F)c(F)c2)C1]

To a stirred solution of anti-methyl 2-(5-(4-(trifluoromethyl)phenyl) piperidin-3-yl) acetate (30.0 mg, 0.10 mmol) in 15 mL of MeOH were added 3-fluoro-4-(trifluoromethyl)benzaldehyde, and AcOH (18.0 mg, 0.30 mmol). After 20 mins, NaBH₃CN (14.0 mg, 0.23 mmol) was added in ice-water bath. The reaction mixture was stirred at room temperature overnight. The reaction mixture was concentrated. The residue was adjusted to pH>7 with aqueous Na₂CO₃ solution and extracted with DCM (20 mL*2). The organic phase was concentrated to give anti-methyl 2-(1-(3-fluoro-4-(trifluoromethyl) benzyl)-5-(4-(trifluoromethyl) phenyl) piperidin-3-yl) acetate (crude, 50 mg, >100% yield) as a yellow solid. LCMS: (Method F) 478.2 (M+H).

### Anti-2-(1-(3-fluoro-4-(trifluoromethyl)benzyl)-5-(4-(trifluoromethyl)phenyl)piperidin-3-yl)acetic acid (130)

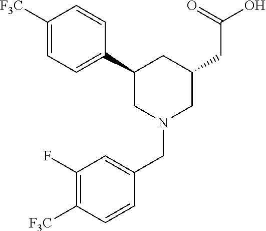 [SMILES: O=C(O)C[C@H]1C[C@H](c2ccc(C(F)(F)F)cc2)CN(Cc2ccc(C(F)(F)F)c(F)c2)C1]

130 was synthesized following the step 7 of scheme 1, substituting anti-methyl 2-(1-(3-fluoro-4-(trifluoromethyl)benzyl)-5-(4-(trifluoromethyl)phenyl) piperidin-3-yl) acetate.

¹H NMR (400 MHz, DMSO-d₆) δ 7.72 (t, J=8.0 Hz, 1H), 7.64 (d, J=8.0 Hz, 2H), 7.57 (d, J=8.0 Hz, 2H), 7.45-7.33 (m, 2H), 3.67-3.49 (m, 2H), 3.16-3.07 (m, 1H), 2.77-2.65 (m, 1H), 2.44-2.36 (m, 3H), 2.36-2.23 (m, 2H), 2.22-2.14 (m, 1H), 1.82-1.72 (m, 1H), 1.72-1.62 (m, 1H). LCMS: (Method F) 464.2 (M+H), Rt. 1.60 min, 97.17% (Max). HPLC: (Method D) Rt. 7.13 min, 99.58% (Max).

### 121

### Anti-2-(-5-(3,4-difluorophenyl)-1-(4-(trifluoromethyl)benzyl)piperidin-3-yl)acetic acid

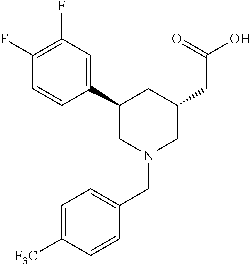 [SMILES: O=C(O)C[C@H]1C[C@H](c2ccc(F)c(F)c2)CN(Cc2ccc(C(F)(F)F)cc2)C1]

121 was synthesized following the route of scheme 1, without chiral separation in step 5, substituting (3,4-difluorophenyl)boronic acid in step 4.

¹H NMR (400 MHz, DMSO-d₆) δ 12.12 (s, 1H), 7.70-7.55 (m, 2H), 7.53-7.46 (m, 2H), 7.44-7.41 (m, 1H), 7.37-7.30 (m, 1H), 7.19 (s, 1H), 3.64-3.52 (m, 2H), 3.05-3.00 (m, 1H), 2.72-2.70 (m, 1H), 2.55-2.52 (m, 1H), 2.39-2.34 (m, 4H), 2.16 (s, 1H), 1.78-1.72 (m, 1H), 1.71-1.62 (m, 1H). LCMS: (Method H) 414.2 (M+H), Rt. 1.66 min, 100.00% (Max). HPLC: (Method E) Rt. 3.42 min, 97.67% (Max).

### 122

### Anti-2-(5-(4-fluorophenyl)-1-(4-(trifluoromethyl)benzyl)piperidin-3-yl)acetic acid

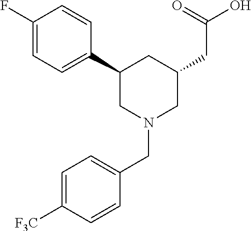 [SMILES: O=C(O)C[C@H]1C[C@H](c2ccc(F)cc2)CN(Cc2ccc(C(F)(F)F)cc2)C1]

122 was synthesized following the route of scheme 1, without chiral separation in step 5, substituting (4-fluorophenyl)boronic acid in step 4.

¹H NMR (400 MHz, DMSO-d₆) δ 12.20 (br.s, 1H), 7.68 (d, J=8.0 Hz, 2H), 7.54 (d, J=8.0 Hz, 2H), 7.35 (t, J=4.0 Hz, 2H), 7.10 (t, J=8.0 Hz, 2H), 3.63-3.51 (m, 2H), 3.05-2.95 (m, 1H), 2.79-2.70 (m, 1H), 2.60-2.54 (m, 1H), 2.48-2.46 (m, 1H), 2.43-2.19 (m, 4H), 1.75-1.61 (m, 2H). LCMS: (Method H) 396.2 (M+H), Rt. 1.59 min, 100.00% (Max). HPLC: (Method E) Rt. 3.41 min, 100.00% (Max).

### 124

### Anti-2-(-1-(3-cyanobenzyl)-5-(4-(trifluoromethyl)phenyl)piperidin-3-yl)acetic acid

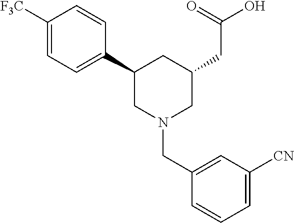 [SMILES: N#Cc1cccc(CN2C[C@@H](CC(=O)O)C[C@H](c3ccc(C(F)(F)F)cc3)C2)c1]

124 was synthesized following the route of scheme 1, without chiral separation in step 5, substituting 3-(bromomethyl)benzonitrile in step 6.

¹H NMR (400 MHz, DMSO-d₆) δ 12.29 (br.s, 1H), 7.77-7.61 (m, 5H), 7.59-7.49 (m, 3H), 3.59 (d, J=13.6 Hz, 1H), 3.50 (d, J=13.6 Hz, 1H), 3.12-3.08 (m, 1H), 2.72 (d, J=9.2 Hz, 1H), 2.62-2.53 (m, 1H), 2.40-2.35 (m, 4H), 2.16 (s, 1H), 1.83-1.75 (m, 1H), 1.68-1.65 (m, 1H). LCMS: (Method E) 403.2 (M+H), Rt. 1.44 min, 100.00% (Max). HPLC: (Method C) Rt. 5.14 min, 99.34% (Max).

### 128

### Anti-2-(1-(2-fluoro-4-(trifluoromethyl)benzyl)-5-(4-(trifluoromethyl)phenyl)piperidin-3-yl)acetic acid

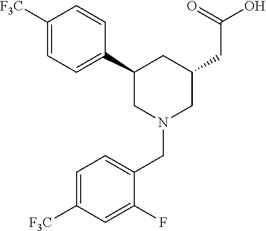 [SMILES: O=C(O)C[C@H]1C[C@H](c2ccc(C(F)(F)F)cc2)CN(Cc2ccc(C(F)(F)F)cc2F)C1]

128 was synthesized following the route of scheme 1, without chiral separation in step 5, substituting 1-(bromomethyl)-2-fluoro-4-(trifluoromethyl)benzene in step 6.

¹H NMR (400 MHz, DMSO-d₆) δ 12.04 (br.s, 1H), 7.74-7.60 (m, 4H), 7.59-7.50 (m, 3H), 3.63 (s, 2H), 3.14-3.03 (m, 1H), 2.80-2.71 (m, 1H), 2.54-2.51 (m, 1H), 2.47-2.26 (m, 4H), 2.21-2.10 (m, 1H), 1.82-1.70 (m, 1H), 1.70-1.58 (m, 1H). LCMS: (Method E) 464.2 (M+H), Rt. 1.55 min, 100.00% (Max). HPLC: (Method D) Rt. 7.49 min, 100.00% (Max).

### 141

### Anti-2-(1-(naphthalen-2-ylmethyl)-5-(4-(trifluoromethyl)phenyl)piperidin-3-yl)acetic acid

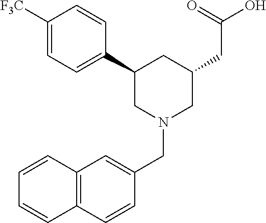 [SMILES: O=C(O)C[C@H]1C[C@H](c2ccc(C(F)(F)F)cc2)CN(Cc2ccc3ccccc3c2)C1]

141 was synthesized following the route of scheme 1, without chiral separation in step 5, substituting 2-(bromomethyl)naphthalene in step 6.

¹H NMR (400 MHz, DMSO-d₆) δ 7.92-7.84 (m, 3H), 7.77 (s, 1H), 7.64-7.57 (m, 4H), 7.55-7.43 (m, 3H), 3.71 (d, J=13.6 Hz, J H), 3.59 (d, J=13.6 Hz, 1H), 3.11 (s, 1H), 2.75 (d, J=8.8 Hz, 1H), 2.50-2.23 (m, 5H), 2.18 (s, 1H), 1.82-1.65 (m, 2H). LCMS: (Method F) 428.2 (M+H), Rt. 1.40 min, 100% (Max). HPLC. (Method C) 428.2 (M+H), Rt. 5.50 min, 99.56% (Max).

### 131

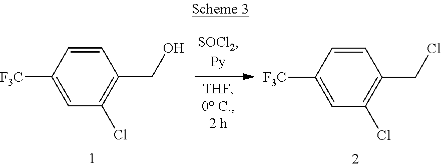 [SMILES: OCc1ccc(C(F)(F)F)cc1Cl ; FC(F)(F)c1ccc(CCl)c(Cl)c1]

### Step 1: 2-chloro-1-(chloromethyl)-4-(trifluoromethyl)benzene

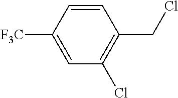 [SMILES: FC(F)(F)c1ccc(CCl)c(Cl)c1]

To a solution of (2-chloro-4-(trifluoromethyl)phenyl)methanol (1.00 g, 4.76 mmol) and Pyr (752 mg, 9.52 mmol) in THF (15 mL) was added sulfurous dichloride (1.12 g, 9.52 mmol) dropwise at ° C. under N₂. The resultant mixture was stirred at 0° C. for 2 h. The mixture was diluted with water (50 mL) and extracted with EA (50 mL×3). The combined organic layer was dried, concentrated to give the title compound. Yield: crude (700 mg, colorless oil). ¹H NMR (400 MHz, CDCl₃) δ 7.68 (d, J=8.0 Hz, 1H), 7.55 (s, 1H), 7.38 (d, J=8.4 Hz, 1H), 4.57 (s, 2H).

### Anti-2-(-1-(2-chloro-4-(trifluoromethyl)benzyl)-5-(4-(trifluoromethyl)phenyl)piperidin-3-yl)acetic acid (131)

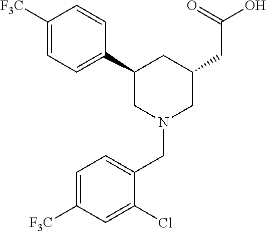 [SMILES: O=C(O)C[C@H]1C[C@H](c2ccc(C(F)(F)F)cc2)CN(Cc2ccc(C(F)(F)F)cc2Cl)C1]

131 was synthesized following the route of scheme 1, without chiral separation in step 5, substituting 2-chloro-1-(chloromethyl)-4-(trifluoromethyl)benzene in step 6.

¹H NMR (400 MHz, DMSO-d₆) δ 7.80 (d, J=8.0 Hz, 1H), 7.65-7.63 (m, 3H), 7.57 (d, J=8.0 Hz, 2H), 7.50 (d, J=8.0 Hz, 1H), 3.63 (d, J=14.4 Hz, 1H), 3.53 (d, J=14.4 Hz, 1H), 3.13-3.08 (m, 1H), 2.75-2.73 (m, 1H), 2.54-2.49 (m, 1H), 2.38-2.32 (m, 4H), 2.18 (s, 1H), 1.83-1.74 (m, 1H), 1.68-1.65 (m, 1H). LCMS: (Method F) 480.2 (M+H), Rt. 1.69 min, 100% (Max). HPLC: (Method D) Rt. 7.53 min, 96.46% (Max).

### 140

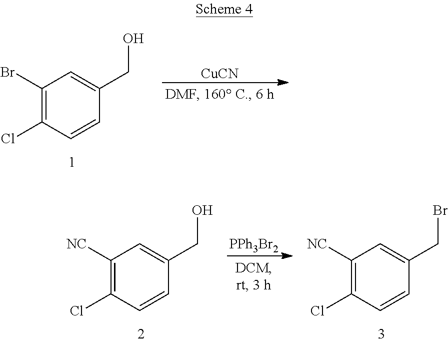

### Step 1: 2-chloro-5-(hydroxymethyl)benzonitrile

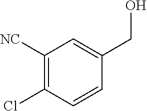 [SMILES: N#Cc1cc(CO)ccc1Cl]

To a solution of (3-bromo-4-chlorophenyl)methanol (222 mg, 1.0 mmol) in DMF (5 mL) was added CuCN (900 mg, 10.0 mmol). The resulting mixture was stirred at 160° C. for 6 h. The reaction mixture was diluted with water (5 mL), and extracted with EtOAc (20 mL×2). The combined organic layers were washed with brine, dried over anhydrous Na₂SO₄, and concentrated under reduced pressure. The residue was purified by flash chromatography on silica gel (EtOAc/PE=1/3) to afford the title compound. Yield: 53% (88.0 mg, colorless oil). ¹H NMR (400 MHz, CDCl₃) δ 7.70-7.69 (m, 1H), 7.55-7.53 (m, 1H), 7.50 (d, J=8.0 Hz, 1H), 4.74 (s, 2H).

### Step 2: 5-(bromomethyl)-2-chlorobenzonitrile

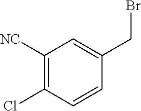 [SMILES: N#Cc1cc(CBr)ccc1Cl]

To a solution of 2-chloro-5-(hydroxymethyl)benzonitrile (88.0 mg, 0.52 mmol) in DCM (3 mL) was added Ph₃PBr₂ (265 mg, 0.63 mmol) in portions. After stirred at room temperature for 3 h, the mixture was concentrated under reduced pressure. The residue was purified by flash chromatography on silica gel (EtOAc/PE=1/10) to afford the title compound. Yield: 33% (40 mg, brown solid). ¹H NMR (400 MHz, CDCl₃) δ 7.70 (d, J=2.0 Hz, 1H), 7.58-7.55 (m, 1H), 7.51 (d, J=8.0 Hz 1H), 4.44 (s, 2H).

### Anti-2-(1-(4-chloro-3-cyanobenzyl)-5-(4-(trifluoromethyl)phenyl)piperidin-3-yl)acetic acid (140)

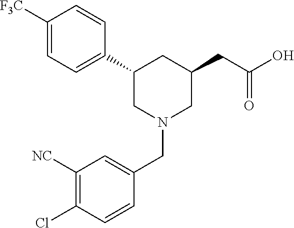 [SMILES: N#Cc1cc(CN2C[C@H](CC(=O)O)C[C@@H](c3ccc(C(F)(F)F)cc3)C2)ccc1Cl]

140 was synthesized following the route of scheme 1, without chiral separation in step 5, using 5-(bromomethyl)-2-chlorobenzonitrile in step 6.

¹H NMR (400 MHz, DMSO-d₆) δ 7.87 (s, 1H), 7.69 (s, 2H), 7.64 (d, J=8.4 Hz, 2H), 7.56 (d, J=8.0 Hz, 2H), 3.57 (d, J=14.0 Hz, 1H), 3.48 (d, J=14.0 Hz, 1H), 3.13-3.07 (m, 1H), 2.74 (d, J=8.0 Hz, 1H), 2.58-2.55 (m, 1H), 2.38-2.33 (m, 4H), 2.17 (s, 1H), 1.81-1.74 (m, 1H), 1.68-1.64 (m, 1H). LCMS: (Method F) 437.0 (M+H), Rt. 1.46 min, 100% (Max). HPLC: (Method D) Rt. 6.52 min, 99.62% (Max).

### 125

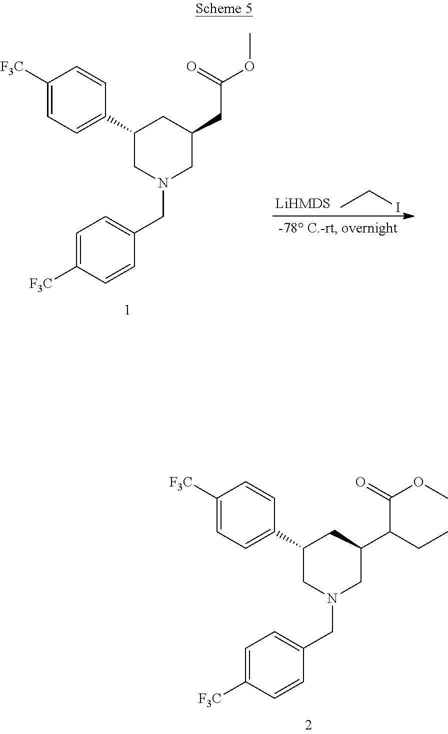

### Step 1: anti-methyl 2-(1-(4-(trifluoromethyl)benzyl)-5-(4-(trifluoromethyl)phenyl)piperidin-3-yl)butanoate

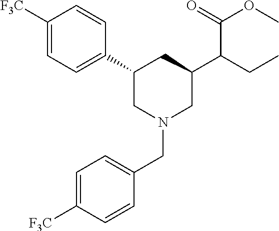 [SMILES: CCC(C(=O)OC)[C@@H]1C[C@@H](c2ccc(C(F)(F)F)cc2)CN(Cc2ccc(C(F)(F)F)cc2)C1]

To a solution of anti-methyl 2-(1-(4-(trifluoromethyl)benzyl)-5-(4-(trifluoromethyl)phenyl)piperidin-3-yl)acetate (250 mg, 0.540 mmol) in THF (15 mL) was added LiHMDS (1.62 mL, 1.62 mmol, 1M in THF) dropwise at −78° C. under N₂. The reaction was stirred at this temperature for 2 h. Iodoethane (253 mg, 1.62 mmol) in THF (2 mL) was added to the above mixture dropwise at −78° C. The resultant mixture was stirred at room temperature for 18 h. The mixture was quenched with sat. aq. NH₄Cl (20 mL) and extracted with EA (15 mL×2). The combined organic layers were washed brine, dried, concentrated, and purified by silica gel column chromatography (PE/EA=20/1-5/1) to give the title compound. Yield: 38% (100 mg, yellow oil). LCMS: (Method F) 488.2 (M+H).

### Anti-2-(1-(4-(trifluoromethyl)benzyl)-5-(4-(trifluoromethyl)phenyl)piperidin-3-yl)butanoic acid (125)

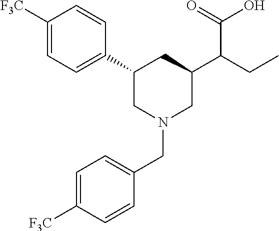 [SMILES: CCC(C(=O)O)[C@@H]1C[C@@H](c2ccc(C(F)(F)F)cc2)CN(Cc2ccc(C(F)(F)F)cc2)C1]

125 was synthesized following step 7 of scheme 1, using anti-methyl 2-(1-(4-(trifluoromethyl)benzyl)-5-(4-(trifluoromethyl)phenyl)piperidin-3-yl)butanoate.

¹H NMR (400 MHz, DMSO-d₆) δ 7.70-7.60 (m, 5H), 7.56-7.50 (m, 3H), 3.67-3.52 (m, 2H), 3.21-3.08 (m, 1H), 2.79-2.67 (m, 2H), 2.39-2.32 (m, 2H), 1.84-1.61 (m, 4H), 1.35-1.24 (m, 2H), 0.81 (t, J=7.2 Hz, 3H). LCMS: (Method F) 474.2 (M+H), Rt. 1.68 min, 100% (Max). HPLC: (Method C) Rt. 6.67 min, 49.37%, 6.80 min, 50.63% (Max).

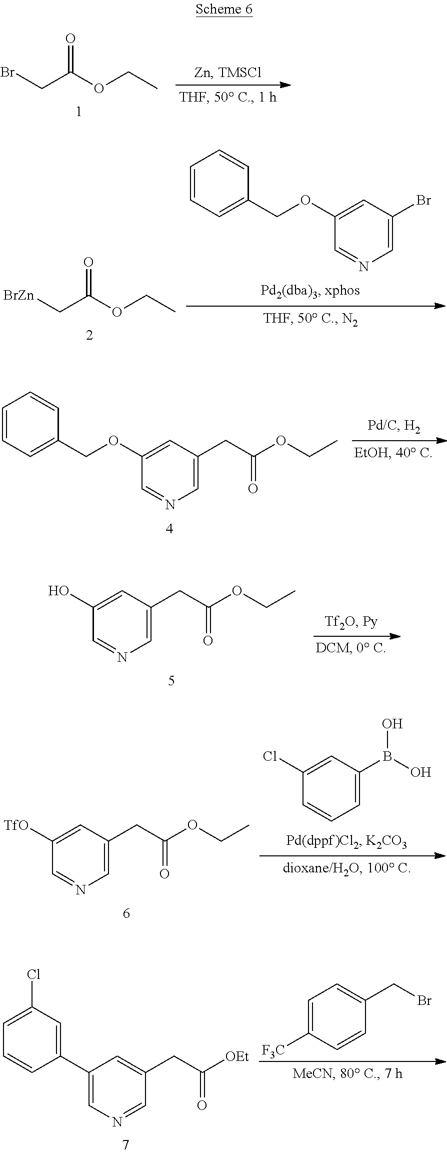

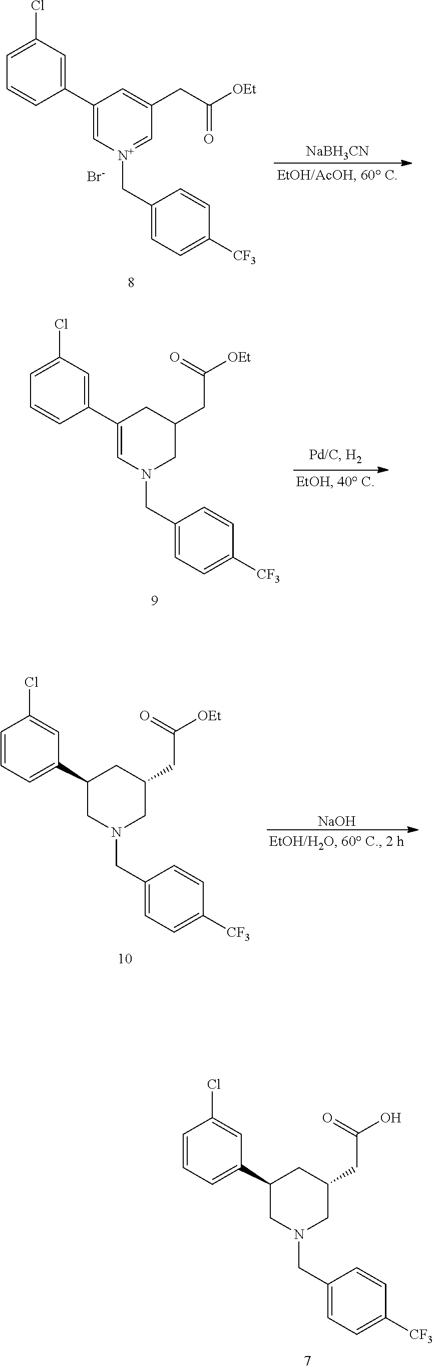

### Step 1: (2-ethoxy-2-oxoethyl)zinc(II) bromide

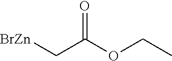

To a mixture of zinc powder (35.0 g, 539 mmol) in dry THF (360 mL) was added TMSCl (3.90 g, 35.9 mmol). After the reaction mixture was stirred at 50° C. under N₂ atmosphere for 0.5 h, ethyl 2-bromoacetate (60.0 g, 359 mmol) was added. The reaction mixture was stirred at 50° C. under N₂ atmosphere for 1.0 h. The light yellow solution was used for subsequent experiments directly.

### Step 2: ethyl 2-(5-(benzyloxy)pyridin-3-yl)acetate

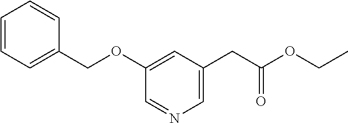 [SMILES: CCOC(=O)Cc1cncc(OCc2ccccc2)c1]

To a solution of 3-(benzyloxy)-5-bromopyridine (29.9 g, 113 mmol), Pd₂(dba)₃ (2.07 g, 2.26 mmol), and Xphos (2.15 g, 4.52 mmol) in THF was added 2-ethoxy-2-oxoethyl)zinc(II) bromide (1 M in THF, 340 mL). The reaction mixture was stirred at 60° C. under N₂. After completion (monitored by TLC and LC-MS), the reaction mixture was poured into ice water. The mixture was filtered through diatomite clay and the filtrate was extracted with EtOAc (3×500 mL). The combined organic layers were washed with brine (2×300 mL), dried over anhydrous sodium sulfate, filtered, and concentrated. The residue was purified by flash chromatography on silica gel to give the title compound. Yield: crude (20.3 g, yellow oil). LCMS: (Method F) 272.1 (M+H).

### Step 3: ethyl 2-(5-hydroxypyridin-3-yl)acetate

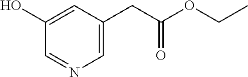

A mixture of ethyl 2-(5-(benzyloxy)pyridin-3-yl)acetate (crude, 12.5 g, 46.1 mmol) and Pd/C (10 wt %, 4.88 g, 4.61 mmol) in EtOH (100 mL) was stirred at 40° C. under N₂ atmosphere. After completion (monitored by TLC and LC-MS), the reaction mixture was filtered, and the filtrate was concentrated under reduced pressure. The residue was purified by flash chromatography on silica gel to give the title compound. Yield: crude (7.4 g, yellow oil). LCMS: (Method F) 182.1 (M+H).

### Step 4: ethyl 2-(5-(((trifluoromethyl)sulfonyl)oxy)pyridin-3-yl)acetate

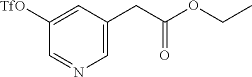 [SMILES: CCOC(=O)Cc1cncc(OS(=O)(=O)C(F)(F)F)c1]

To a solution of ethyl 2-(5-hydroxypyridin-3-yl)acetate (10.0 g, 55.2 mmol) and pyridine (13.1 g, 166 mmol) in DCM (120 mL) was added Tf₂O (17.1 g, 60.7 mmol) at 0° C. The reaction mixture was stirred at room temperature. After completion (monitored by TLC and LC-MS), the reaction mixture was washed with brine (2×20 mL), dried over anhydrous Na₂SO₄, filtered, and concentrated. The residue was purified by flash chromatography on silica gel to give the title compound. Yield: 40.5% (7.00 g, yellow oil). LCMS: (Method F) 314.0 (M+H).

### Step 5: ethyl 2-(5-(3-chlorophenyl)pyridin-3-yl)acetate

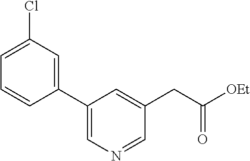 [SMILES: CCOC(=O)Cc1cncc(-c2cccc(Cl)c2)c1]

A solution of ethyl 2-(5-(((trifluoromethyl)sulfonyl)oxy)pyridin-3-yl)acetate (700 mg, 2.23 mmol), (3-chlorophenyl)boronic acid (2.46 mmol), Pd(dppf)Cl₂ (23.0 mg, 0.032 mmol), and K₂CO₃ (440 mg, 3.19 mmol) in dioxane/H₂O (10 mL/2 mL) was stirred at 100° C. under N₂ atmosphere. After completion (monitored by LC-MS), organic solvent was removed under vacuum and the residue was extracted with EtOAc (3×10 mL). The combined organic layers were washed with brine (2×10 mL), dried over anhydrous sodium sulfate, filtered, and concentrated. The residue was purified by flash chromatography on silica gel to give the title compound.

### Step 6: 3-(3-chlorophenyl)-5-(2-ethoxy-2-oxoethyl)-1-(4-(trifluoromethyl)benzyl) pyridin-1-ium bromide

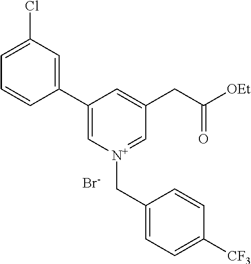 [SMILES: CCOC(=O)Cc1cc(-c2cccc(Cl)c2)c[n+](Cc2ccc(C(F)(F)F)cc2)c1 ; [Br-]]

A solution of ethyl 2-(5-(3-chlorophenyl)pyridin-3-yl)acetate (1.96 mmol) and 1-(bromomethyl)-4-(trifluoromethyl)benzene (561 mg, 2.35 mmol) in MeCN (5 mL) was stirred at 80° C. for 7 hours. After completion (monitored by TLC), the reaction mixture was concentrated under vacuum and the residue was purified by flash chromatography on silica gel to give the title compound.

### Step 7: ethyl 2-(5-(3-chlorophenyl)-1-(4-(trifluoromethyl)benzyl)-1,2,3,4-tetrahydropyridin-3-yl)acetate

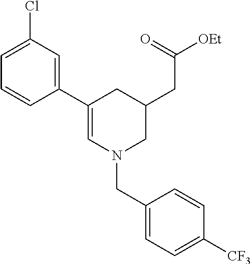 [SMILES: CCOC(=O)CC1CC(c2cccc(Cl)c2)=CN(Cc2ccc(C(F)(F)F)cc2)C1]

To a solution of 3-(3-chlorophenyl)-5-(2-ethoxy-2-oxoethyl)-1-(4-(trifluoromethyl)benzyl)pyridin-1-ium bromide (1.03 mmol) in EtOH/AcOH (10 mL/1 mL) was added NaBH₃CN (649 mg, 10.3 mmol) in batches at room temperature. The reaction mixture was stirred at 60° C. After completion (monitored by LC-MS), the reaction mixture was neutralized with 10% aq. Na₂CO₃ solution. The mixture was extracted with DCM (3×10 mL). The combined organic layers were washed with brine (2×10 mL), dried over anhydrous sodium sulfate, filtered, and concentrated to get the crude product.

### Step 8: anti-ethyl 2-5-(3-chlorophenyl)-1-(4-(trifluoromethyl)benzyl)piperidin-3-yl) acetate

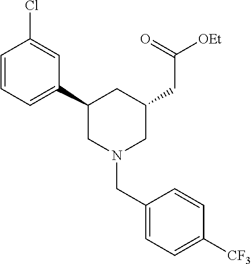 [SMILES: CCOC(=O)C[C@H]1C[C@H](c2cccc(Cl)c2)CN(Cc2ccc(C(F)(F)F)cc2)C1]

A mixture of ethyl 2-(5-(3-chlorophenyl)-1-(4-(trifluoromethyl)benzyl)-1,2,3,4-tetrahydropyridin-3-yl) acetate (crude, 1.23 mmol) and Pd/C (10 wt %, 390 mg) in EtOH (10 mL) was stirred under H₂ at 40° C. After completion (monitored by LCMS), the reaction mixture was filtered, and the filtrate was concentrated under vacuum. The residue was purified by prep-HPLC to give the title compound.

### Step 9: anti-2-(S5-(3-chlorophenyl)-1-(4-(trifluoromethyl)benzyl)piperidin-3-yl)acetic acid (143)

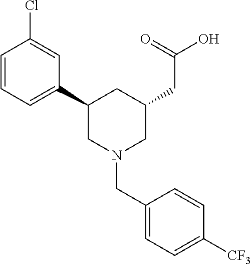 [SMILES: O=C(O)C[C@H]1C[C@H](c2cccc(Cl)c2)CN(Cc2ccc(C(F)(F)F)cc2)C1]

A solution of anti-ethyl 2-(5-(3-chlorophenyl)-1-(4-(trifluoromethyl)benzyl) piperidin-3-yl) acetate (0.050 mmol) and NaOH (12.0 mg, 0.31 mmol) in EtOH/H₂O (2.5 mL/0.5 mL) was stirred at 60′C for 2 hours. After completion (monitored by LC-MS), EtOH was removed under vacuum. The residue was acidified with aq. HCl (1 N) to pH 6-7. The resulting suspension was extracted with DCM (2×1 mL). The combined organic layer was washed with brine (2×2 mL), dried over anhydrous sodium sulfate, and concentrated under vacuum. The residue was purified by prep-HPLC to give the title compound.

¹H NMR (400 MHz, DMSO-d₆) δ 7.67 (d, J=8.0 Hz, 2H), 7.54 (d, J=8.0 Hz, 2H), 7.42 (s, 1H), 7.35-7.19 (m, 3H), 3.62 (d, J=12.0 Hz, 1H), 3.52 (d, J=16.0 Hz, 1H), 3.06-2.95 (m, 1H), 2.76-2.63 (m, 1H), 2.57-2.51 (m, 2H), 2.42-2.31 (m, 3H), 2.21-2.08 (m, 1H), 1.81-1.69 (m, 1H), 1.68-1.56 (m, 1H). LCMS: (Method F) 412.0 (M+H), Rt. 1.42 min, 99.07% (Max). HPLC: (Method C) Rt. 5.76 min, 98.14% (Max).

### 138

### Anti-2-(1-benzyl-5-(4-(trifluoromethyl)phenyl)piperidin-3-yl)acetic acid

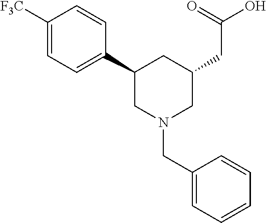 [SMILES: O=C(O)C[C@H]1C[C@H](c2ccc(C(F)(F)F)cc2)CN(Cc2ccccc2)C1]

138 was synthesized following the route of scheme 6, substituting (bromomethyl)benzene in step 7. ¹H NMR (400 MHz, DMSO-d₆) δ 12.08 (s, 1H), 7.64 (d, J=8.4 Hz, 2H), 7.57 (d, J=8.0 Hz, 2H), 7.35-7.28 (m, 4H), 7.27-7.20 (m, 1H), 3.53 (d, J=13.6 Hz, 1H), 3.44 (d, J=13.6 Hz, 1H), 3.13-3.03 (m, 1H), 2.74-2.64 (m, 1H), 2.47-2.28 (m, 5H), 2.14 (s, 1H), 1.83-1.60 (m, 2H). LCMS: (Method F) 378.2 (M+H), Rt. 1.31 min, 100/6 (Max). HPLC: (Method C) 378.2 (M+H), Rt. 4.84 min, 100% (Max).

### 135

### Anti-2-(1-(4-chlorobenzyl)-5-(4-chlorophenyl)piperidin-3-yl)acetic acid

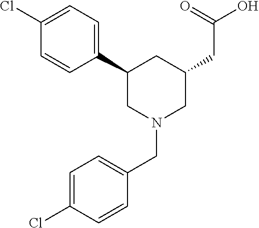 [SMILES: O=C(O)C[C@H]1C[C@H](c2ccc(Cl)cc2)CN(Cc2ccc(Cl)cc2)C1]

135 was synthesized following the route of scheme 6, substituting methyl 2-(5-bromopyridin-3-yl)acetate and 2-(4-chlorophenyl)-4,4,5,5-tetramethyl-1,3,2-dioxaborolane in step 6, and 1-(bromomethyl)-4-chlorobenzene in step 7. ¹H NMR (400 MHz, DMSO-d₆) δ 7.42-7.26 (m, 8H), 3.52-3.41 (m, 2H), 3.01-2.92 (m, 1H), 2.69-2.62 (m, 1H), 2.47-2.19 (m, 5H), 2.19-2.12 (m, 1H), 1.73-1.58 (m, 2H). LCMS: (Method E) 378.2, 380.2 (M+H), Rt. 1.25 min, 100.00% (Max). HPLC: (Method D) Rt. 6.94 min, 100.00% (Max).

### 139

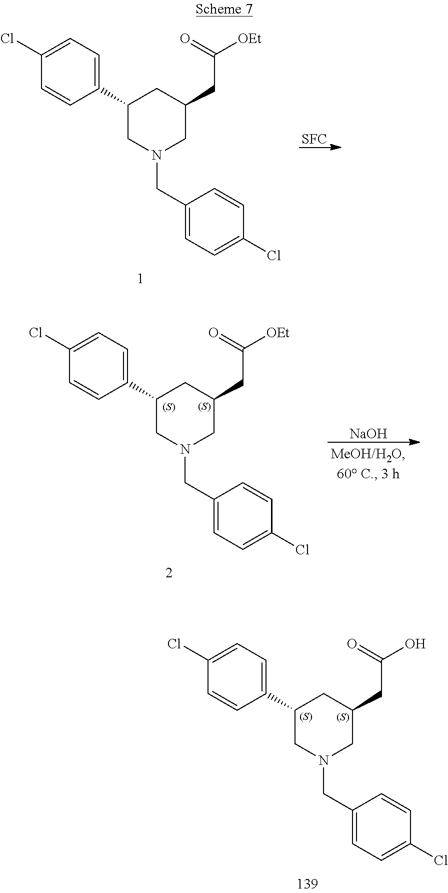

### Step 1: ethyl 2-((3R,5R)-1-(4-chlorobenzyl)-5-(4-chlorophenyl)piperidin-3-yl) acetate

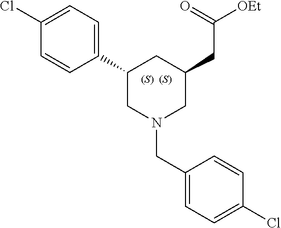

Anti-ethyl 2-(1-(4-chlorobenzyl)-5-(4-chlorophenyl)piperidin-3-yl) acetate (intermediate of 135) was purified via Chiral SFC to give the title compound as fraction 2. Chiral SFC: (Method H). Rt. 4.306 min, 49.65% (Max) and Rt. 4.808 min, 50.35% (Max). Yield: 47.8% (45.0 mg, white solid). LCMS: (Method F) 406.1 (M+H), Rt. 1.31 min, 97.21% (Max). Chiral SFC: (Method H) Rt. 4.768 min, 99.69% (Max).

### Step 2: 2-((3S,5S)-1-(4-chlorobenzyl)-5-(4-chlorophenyl)piperidin-3-yl)acetic acid

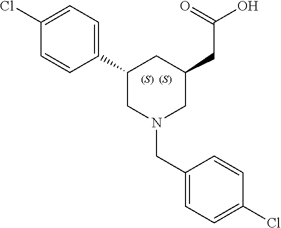

Ethyl 2-((3S,5S)-1-(4-chlorobenzyl)-5-(4-chlorophenyl)piperidin-3-yl) acetate (45.0 mg, 0.11 mmol) in MeOH (3 mL) was added NaOH (24.0 mg, 0.60 mmol) in water (0.5 mL). The mixture was stirred at 60° C. for 3 h. The mixture was neutralized with HCl (1 M) to pH=5 and extracted with EtOAc (10 mL×2). The combined organic layers were dried over anhydrous Na₂SO₄, concentrated, and purified by prep-HPLC to afford title compound. Prep-HPLC: (Method C). Yield: 50% (20.9 mg, white solid). ¹H NMR (400 MHz, DMSO-d₆) δ 7.39-7.34 (m, 4H), 7.30-7.25 (m, 4H), 3.82-3.71 (m, 2H), 3.17-3.10 (m, 1H), 3.02-2.99 (m, 1H), 2.91 (d, J=8.0 Hz, 1H), 2.66-2.49 (m, 4H), 2.39-2.32 (m, 1H), 1.92-1.79 (m, 2H). LCMS: (Method F) 378.2 (M+H), Rt. 1.30 min, 100% (Max). HPLC: (Method D) Rt. 6.78 min, 100% (Max).

### 123

### Anti-2-(-5-(4-chlorophenyl)-1-(4-(trifluoromethyl)benzyl)piperidin-3-yl)acetic acid

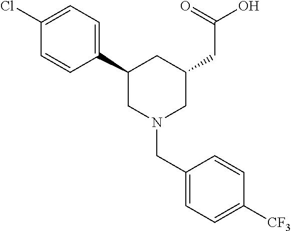 [SMILES: O=C(O)C[C@H]1C[C@H](c2ccc(Cl)cc2)CN(Cc2ccc(C(F)(F)F)cc2)C1]

123 was synthesized following the route of scheme 6, substituting (4-chlorophenyl)boronic acid in step 6. ¹H NMR (400 MHz, DMSO-d₆) δ 12.05 (br.s, 1H), 7.68 (d, J=8.0 Hz, 2H), 7.54 (d, J=8.0 Hz, 2H), 7.41-7.27 (m, 4H), 3.63-3.51 (m, 2H), 3.00 (s, 1H), 2.75-2.67 (m, 1H), 2.46-2.10 (m, 6H), 1.72-1.62 (m, 2H). LCMS: (Method F) 412.2 (M+H), Rt. 1.40 min, 97.0% (Max). HPLC: (Method D) Rt. 6.99 min, 99.9% (Max).

### 141

### 2-((3S,5S)-5-(4-chlorophenyl)-1-(4-(trifluoromethyl)benzyl)piperidin-3-yl)acetic acid

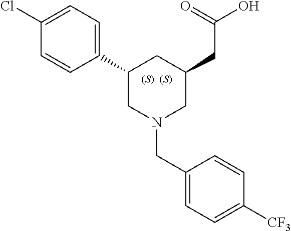

Anti-2-(5-(4-chlorophenyl)-1-(4-(trifluoromethyl)benzyl)piperidin-3-yl)acetic acid (123) was further purified by Chiral SFC purification to give the title compound as fraction 2. Chiral SFC: (Method F) Rt. 1.419 min, 49.61% (Max) and Rt. 1.999 min, 49.09% (Max). Yield: 28% (30 mg, white solid). ¹H NMR (400 MHz, DMSO-d₆) δ 7.67 (d, J=8.0 Hz, 2H), 7.53 (d, J=8.0 Hz, 2H), 7.38-7.29 (m, 4H), 3.61 (d, J=12.0 Hz, 1H), 3.52 (d, J=16.0 Hz, 1H), 3.03-2.95 (m, 1H), 2.74-2.66 (m, J H), 2.58-2.51 (m, 2H), 2.40-2.36 (m, 1H), 2.35-2.28 (m, 2H), 2.16 (s, 1H), 1.75-1.60 (m, 2H). LCMS: (Method F) 412.0 (M+H), Rt. 1.43 min, 99.17% (Max). HPLC: (Method D) Rt. 7.10 min, 98.51% (Max). Chiral SFC: (Method F) Rt. 1.974 min, 100% (Max).

### 133

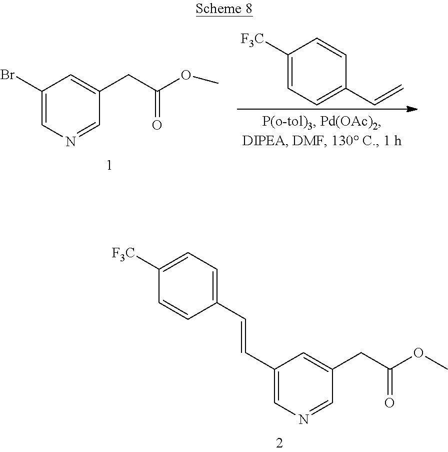 [SMILES: COC(=O)Cc1cncc(Br)c1 ; C=Cc1ccc(C(F)(F)F)cc1 ; COC(=O)Cc1cncc(/C=C/c2ccc(C(F)(F)F)cc2)c1]

### Step 1: methyl (E)-2-(5-(4-(trifluoromethyl)styryl)pyridin-3-yl)acetate

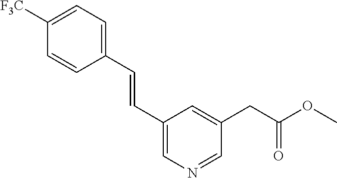 [SMILES: COC(=O)Cc1cncc(/C=C/c2ccc(C(F)(F)F)cc2)c1]

To a solution of methyl 2-(5-bromopyridin-3-yl)acetate (550 mg, 2.40 mmol) in DMF (8 mL) were added 1-(trifluoromethyl)-4-vinylbenzene (826 mg, 4.80 mmol) and DIPEA (1.2 mL). Then P(o-tol)₃ (146 mg, 0.480 mmol) and palladium acetate (53.0 mg, 0.240 mmol) were added. The reaction mixture was stirred at 130° C. for 1 hour after nitrogen degassing. The mixture was diluted with water, and extracted with DCM (20 mL*3). The organic layer was washed with water and brine, dried over Na₂SO₄ and concentrated in vacuum. The mixture was purified via silica gel column (PE:EA=2:1) to give the title compound. Yield: 74% (570 mg, yellow oil). LCMS: (Method E) 322.10 (M+H).

### Anti-2-(1-(4-(trifluoroethyl)benzyl)-5-(4-(trifluoromethyl)phenethyl)piperidin-3-yl)acetic acid

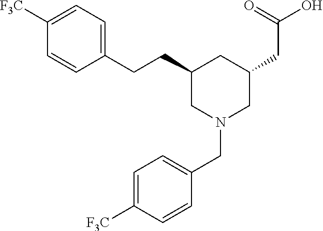 [SMILES: O=C(O)C[C@H]1C[C@H](CCc2ccc(C(F)(F)F)cc2)CN(Cc2ccc(C(F)(F)F)cc2)C1]

133 was synthesized following the route of scheme 6, using methyl (E)-2-(5-(4-(trifluoromethyl)styryl)pyridin-3-yl)acetate in step 7.

¹H NMR (400 MHz, CD₃OD) δ 7.81 (d, J=8.0 Hz, 2H), 7.70 (d, J=8.0 Hz, 2H), 7.57 (d, J=8.0 Hz, 2H), 7.38 (d, J=8.0 Hz, 2H), 4.44 (d, J=13.2 Hz, 1H), 4.37 (d, J=12.8 Hz, 1H), 3.66-3.32 (m, 2H), 3.19-3.03 (m, 1H), 2.86-2.63 (m, 3H), 2.59-2.31 (m, 3H), 2.08-1.93 (m, 2H), 1.69-1.47 (m, 3H). LCMS: (Method E) 474.3 (M+H), Rt. 1.97 min, 100% (Max). HPLC: (Method D) 474.3 (M+H), Rt. 8.04 min, 99.64% (Max).

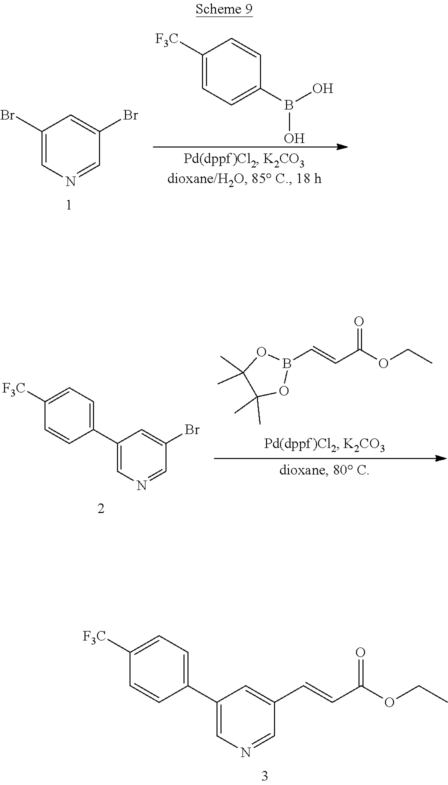 [SMILES: Brc1cncc(Br)c1 ; OB(O)c1ccc(C(F)(F)F)cc1 ; FC(F)(F)c1ccc(-c2cncc(Br)c2)cc1 ; CCOC(=O)/C=C/B1OC(C)(C)C(C)(C)O1 ; CCOC(=O)/C=C/c1cncc(-c2ccc(C(F)(F)F)cc2)c1]

### Step 1: 3-bromo-5-(4-(trifluoromethyl)phenyl)pyridine

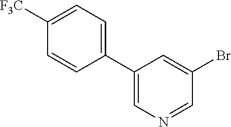 [SMILES: FC(F)(F)c1ccc(-c2cncc(Br)c2)cc1]

To a mixture of 3,5-dibromopyridine (5.00 g, 23.3 mmol), (4-(trifluoromethyl)phenyl)boronic acid (3.65 g, 19.2 mmol) and K₂CO₃ (5.87 g, 42.6 mmol) in dioxane/H₂O (100 mL, V/V=10:1) was added Pd(dppf)Cl₂ (867 mg, 1.06 mmol) under N₂. The resultant mixture was stirred at 85° C. for 18 h. The mixture was diluted with water (100 mL) and extracted with EtOAc (50 mL×3). The combined organic layer was dried, concentrated, and purified by silica gel column (PE/EA=20/1-2:1) to give the title compound. Yield: 44% (2.80 g, white solid). ¹H NMR (400 MHz, CDCl₃) δ 8.77 (d, J=2.0 Hz, 1H), 8.72 (d, J=2.0 Hz, 1H), 8.04 (t, J=4.0 Hz, 1H), 7.76 (d, J=8.0 Hz, 2H), 7.68 (d, J=8.0 Hz, 2H). LCMS: (Method H) 302.2 (M+H).

### Step 2: ethyl (E)-3-(5-(4-(trifluoromethyl)phenyl)pyridin-3-yl)acrylate

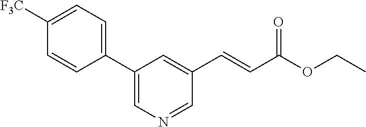 [SMILES: CCOC(=O)/C=C/c1cncc(-c2ccc(C(F)(F)F)cc2)c1]

To a mixture of 3-bromo-5-(4-(trifluoromethyl)phenyl)pyridine (1.00 g, 3.30 mmol), ethyl (E)-3-(4,4,5,5-tetramethyl-1,3,2-dioxaborolan-2-yl)acrylate (1.18 g, 4.95 mmol) and K₂CO₃ (910 mg, 6.60 mmol) in dioxane/H₂O (30 ml, V:V=10:1) was added Pd(dppf)Cl₂ (134 mg, 0.170 mmol). The resultant mixture was stirred at 85° C. for 18 h under N₂. The mixture was diluted with water (100 mL) and extracted with EtOAc (50 mL×3). The combined organic layer was dried, concentrated, and purified by flash chromatography on silica gel (PE/EA=20/1-1:1) to give the title compound. Yield: 77% (820 mg, white solid). ¹H NMR (400 MHz, CDCl₃) δ 8.84 (d, J=2.0 Hz, 1H), 8.78 (d, J=2.0 Hz, 1H), 8.01 (t, J=1.6 Hz, 1H), 7.78-7.70 (m, 5H), 6.60 (d, J=16.4 Hz, 1H), 4.30 (q, J=8.0 Hz, 2H), 1.36 (t, J=8.0 Hz, 3H). LCMS: (Method F) 322.1 (M+H).

### Anti-3-(1-(4-(trifluoromethyl)benzyl)-5-(4-(trifluoromethyl)phenyl)piperidin-3-yl)propanoic acid (126)

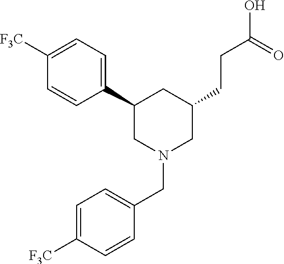 [SMILES: O=C(O)CC[C@H]1C[C@H](c2ccc(C(F)(F)F)cc2)CN(Cc2ccc(C(F)(F)F)cc2)C1]

126 was synthesized following the route of scheme 6, using ethyl (E)-3-(5-(4-(trifluoromethyl)phenyl)pyridin-3-yl)acrylate in step 6. ¹H NMR (400 MHz, DMSO-d₆) δ 12.11 (br.s, 1H), 7.68 (d, J=8.0 Hz, 2H), 7.63 (d, J=8.0 Hz, 2H), 7.56 (t, J=8.0 Hz, 4H), 3.58 (q, J=13.2 Hz, 2H), 3.14-3.06 (m, 1H), 2.72-2.70 (m, 1H), 2.42-2.33 (m, 3H), 2.15 (t, J=7.2 Hz, 2H), 1.81-1.57 (m, 5H). LCMS: (Method F) 480.2 (M+H), Rt. 1.44 min, 95.74% (Max). HPLC: (Method D) Rt. 7.60 min, 98.0% (Max).

### 136 & 146

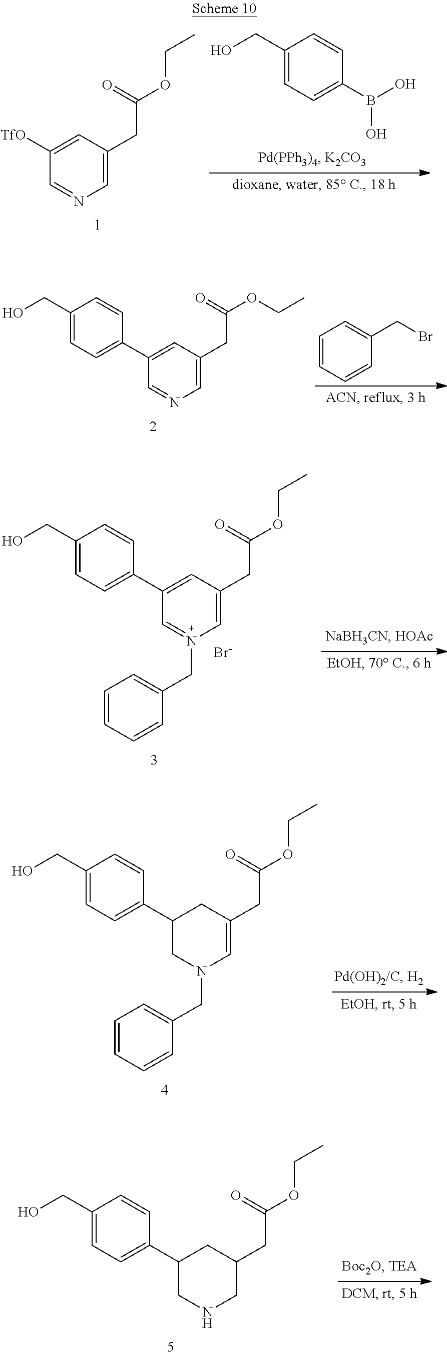

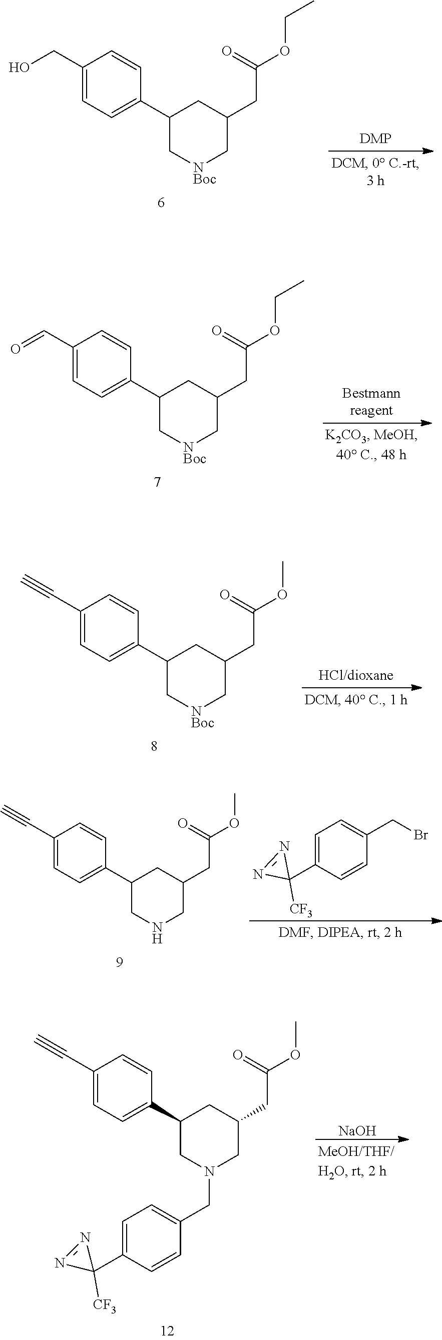

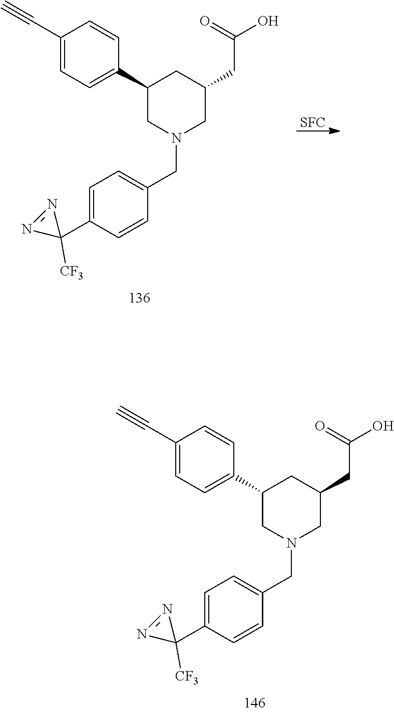 [SMILES: C#Cc1ccc([C@H]2C[C@H](CC(=O)O)CN(Cc3ccc(C4(C(F)(F)F)N=N4)cc3)C2)cc1 ; "SFC" ; C#Cc1ccc([C@@H]2C[C@@H](CC(=O)O)CN(Cc3ccc(C4(C(F)(F)F)N=N4)cc3)C2)cc1]

### Step 1: ethyl 2-(5-(4-(hydroxymethyl)phenyl)pyridin-3-yl)acetate

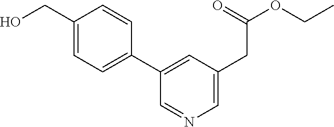 [SMILES: CCOC(=O)Cc1cncc(-c2ccc(CO)cc2)c1]

To a mixture of ethyl 2-(5-(((trifluoromethyl)sulfonyl)oxy)pyridin-3-yl)acetate (4.00 g, 14.1 mmol), (4-(hydroxymethyl)phenyl)boronic acid (4.10 g, 28.0 mmol) and K₂CO₃ (3.86 g, 28.0 mmol) in dioxane/H₂O (60 mL, V:V=10:1) was added Pd(dppf)Cl₂ (1.00 g, 0.140 mmol). The mixture was degassed and refilled with N₂ for 3 times. The resultant mixture was stirred at 85° C. for 18 h under N₂. The mixture was diluted with water (100 mL) and extracted with EtOAc (50 mL×3). The combined organic layer was dried, concentrated, and purified by flash chromatography on silica gel (PE/EA=20/1-1:3) to give the title compound. Yield: 78% (3.20 g, yellow liquid). LCMS: (Method H) 272.1 (M+H).

### Step 2: 1-benzyl-3-(2-ethoxy-2-oxoethyl)-5-(4-(hydroxymethyl)phenyl)pyridin-1-ium bromide

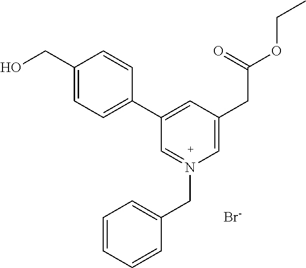

A mixture of ethyl 2-(5-(4-(hydroxymethyl)phenyl)pyridin-3-yl)acetate (3.20 g, 18.8 mmol) and (bromomethyl)benzene (2.17 g, 12.7 mmol) in MeCN (50 mL) was refluxed for 3 h. The precipitate was collected by filtration, washed with Et₂O and dried to give the title compound, which was used for next step without further purification. Yield: 70% (3.30 g, yellow solid). LCMS: (Method H) 362.1 (M+H).

### Step 3: ethyl 2-(1-benzyl-5-(4-(hydroxymethyl)phenyl)-1,4,5,6-tetrahydropyridin-3-yl)acetate

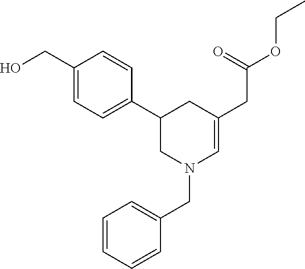 [SMILES: CCOC(=O)CC1=CN(Cc2ccccc2)CC(c2ccc(CO)cc2)C1]

To a mixture of 1-benzyl-3-(2-ethoxy-2-oxoethyl)-5-(4-(hydroxymethyl)phenyl)pyridin-1-ium bromide (3.20 g, 8.80 mmol) and HOAc (3 mL) in EtOH (30 mL) was added NaBH₃CN (2.78 g, 44.1 mmol) in portions at room temperature. The reaction was stirred at 70° C. for 6 h. LCMS showed the reaction worked well. The mixture was diluted with saturated K₂CO₃ aqueous solution (100 mL) and extracted with EtOAc (50 mL×3). The combined organic layer was washed with brine, dried over Na₂SO₄, and concentrated to give the title compound, which was used for next step without further purification. Yield: crude (3.80 g, yellow oil). LCMS: (Method H) 366.1 (M+H).

### Step 4: ethyl 2-(5-(4-(hydroxymethyl)phenyl)piperidin-3-yl)acetate

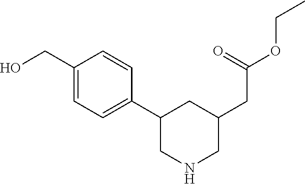 [SMILES: CCOC(=O)CC1CNCC(c2ccc(CO)cc2)C1]

A mixture of ethyl 2-(1-benzyl-5-(4-(hydroxymethyl)phenyl)-1,4,5,6-tetrahydropyridin-3-yl)acetate (crude, 2.70 g, 7.30 mmol), HOAc (1 mL) and Pd(OH)₂ (270 mg, 10% on carbon) in EtOH was stirred at room temperature under H₂ (15 psi) for 5 h. The mixture was filtered and the filtrate was concentrated to give the title compound, which was used for next step without further purification. Yield: crude (4.50 g, yellow oil). LCMS: (Method H) 278.1 (M+H).

### Step 5: tert-butyl 3-(2-ethoxy-2-oxoethyl)-5-(4-(hydroxymethyl)phenyl)piperidine-1-carboxylate

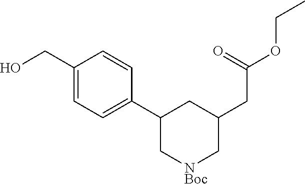 [SMILES: CCOC(=O)CC1CC(c2ccc(CO)cc2)CN(C(=O)OC(C)(C)C)C1]

To a solution of ethyl-2-(5-(4-(hydroxymethyl)phenyl)piperidin-3-yl)acetate (900 mg, crude) and di-tert-butyl dicarbonate (520 mg, 2.38 mmol) in DCM (15 mL) was added TEA (725 mg, 7.16 mmol). The reaction was stirred at room temperature for 5 h. After completion (monitored by LCMS), the mixture was diluted with water (20 mL) and extracted with EtOAc (15 mL×2). The combined organic layer was dried, concentrated, and purified by silica gel column (PE/EA=50/1-10:1) to give the title compound. Yield: 65% (700 mg, yellow liquid). LCMS: (Method H) 378.2 (M+H).

### Step 6: tert-butyl 3-(2-ethoxy-2-oroethyl)-5-(4-formylphenyl)piperidine-1-carboxylate

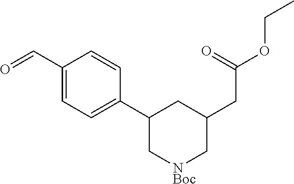 [SMILES: CCOC(=O)CC1CC(c2ccc(C=O)cc2)CN(C(=O)OC(C)(C)C)C1]

To a solution of 3-(2-ethoxy-2-oxoethyl)-5-(4-(hydroxymethyl)phenyl)piperidine-1-carboxylate (700 mg, 1.85 mmol)) in DCM (15 mL) was added and Dess-Martin (1.20 g, 2.78 mmol) in one portion at 0° C. and stirred at room temperature for 3 h. After completion (monitored by TLC), the mixture was concentrated to give the crude, which was purified by Flash chromatography on silica gel column (PE/EA=50/1-10:1) to give the title compound. Yield: 76% (530 mg, white solid). LCMS: (Method H) 376.2 (M+H), 92% (Max).

### Step 7: tert-butyl 3-(4-ethynylphenyl)-5-(2-methoxy-2-oxoethyl)piperidine-1-carboxylate

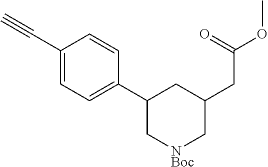 [SMILES: C#Cc1ccc(C2CC(CC(=O)OC)CN(C(=O)OC(C)(C)C)C2)cc1]

To a solution of tert-butyl 3-(2-ethoxy-2-oxoethyl)-5-(4-formylphenyl)piperidine-1-carboxylate (530 mg, crude) and Bestmann-Ohira reagent (407 mg, 2.12 mmol) in MeOH (15 mL) was added K₂CO₃ (725 mg, 2.12 mmol). The reaction was stirred at 40° C. for 48 h. After completion (monitored by LCMS), the mixture was concentrated, and purified by Flash chromatography on silica gel (PE/EA=50/1-10:1) to give the title compound. Yield: 66% (350 mg, white solid). LCMS: (Method H) 358.2 (M+H).

### Step 8: methyl 2-(5-(4-ethynylphenyl)piperidin-3-yl)acetate

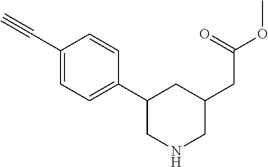 [SMILES: C#Cc1ccc(C2CNCC(CC(=O)OC)C2)cc1]

To a solution of tert-butyl 3-(4-ethynylphenyl)-5-(2-methoxy-2-oxoethyl)piperidine-1-carboxylate (350 mg, 0.98 mmol) in DCM (10 mL) was added dioxane/HCl (3 mL, 2 M). The reaction was stirred at 40° C. for 1 h. After completion (monitored by LCMS), the mixture was concentrated to give the title compound, which was used for next step without further purification. Yield: 100% (265 mg, white solid, HCl salt). LCMS: (Method F) 258.1 (M+H).

### Step 9: anti-methyl 2-(5-(4-ethynylphenyl)-1-(4-(3-(trifluoromethyl)-3H-diazirin-3-yl)benzyl)piperidin-3-yl)acetate

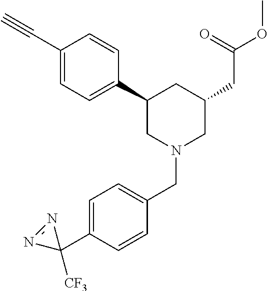 [SMILES: C#Cc1ccc([C@H]2C[C@H](CC(=O)OC)CN(Cc3ccc(C4(C(F)(F)F)N=N4)cc3)C2)cc1]

To a solution of methyl 2-(5-(4-ethynylphenyl)piperidin-3-yl)acetate (265 mg, 1.00 mmol) and 3-(4-(bromomethyl)phenyl)-3-(trifluoromethyl)-3H-diazirine (336 mg, 1.20 mmol) in DMF (10 mL) was added DIPEA (387 mg, 3.00 mmol). The reaction was stirred at room temperature for 2 h. After completion (monitored by LCMS), the mixture was diluted with water (30 mL) and extracted with EtOAc (15 mL×2). The combined organic layer was dried, concentrated, and purified by Flash chromatography on silica gel (PE/EA=20/1-5:1) to give the title compounds. Non-polar isomer (minor isomer, assigned as anti). Yield: 15% (70.0 mg, white solid). LCMS: (Method F) 456.2 (M+H), Rt. 1.72 min, 90% (Max).

### Step 10: anti-2-(5-(4-ethynylphenyl)-1-(4-(3-(trifluoromethyl)-3H-diazirin-3-yl)benzyl)piperidin-3-yl)acetate

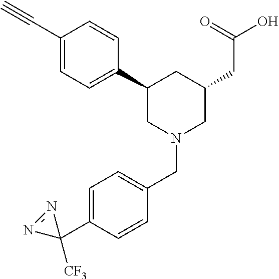 [SMILES: C#Cc1ccc([C@H]2C[C@H](CC(=O)O)CN(Cc3ccc(C4(C(F)(F)F)N=N4)cc3)C2)cc1]

To a solution of anti-methyl 2-(5-(4-ethynylphenyl)-1-(4-(3-(trifluoromethyl)-3H-diazirin-3-yl)benzyl)piperidin-3-yl)acetate (70.0 mg, 0.154 mmol) in MeOH/THF/H₂O (3:3:1, 10 mL) was added NaOH (18.4 mg, 0.460 mmol) at room temperature. The mixture was stirred at room temperature for 2 h. After completion (the reaction mixture was monitored by TLC), the organic solvent was removed under vacuum. The residue was acidified with aq. HCl (3 N) to pH 4˜5. The precipitated solid was filtered and purified to give the title compound. Prep-HPLC: (Method C). Yield: 57% (40 mg, white solid). ¹H NMR (400 MHz, DMSO-d₆) δ 12.10 (br.s, 1H), 7.44 (d, J=8.4 Hz, 2H), 7.38 (d, J=8.0 Hz, 2H), 7.32 (d, J=8.0 Hz, 2H), 7.22 (d, J=8.0 Hz, 2H), 4.12 (s, 1H), 3.55 (d, J=14.0 Hz, 1H), 3.47 (d, J=14.0 Hz, 1H), 3.03-2.92 (m, 1H), 2.69-2.67 (m, 1H), 2.53-2.15 (m, 6H), 1.74-1.61 (m, 2H). LCMS: (Method F) 442.2 (M+H), Rt. 1.38 min, 100% (Max). HPLC: (Method C) Rt. 5.72 min, 99.98% (Max).

### Step 12: 2-((3S,5S)-5-(4-ethynylphenyl)-1-(4-(3-(trifluoromethyl)-3H-diazirin-3-yl)benzyl)piperidin-3-yl)acetic acid

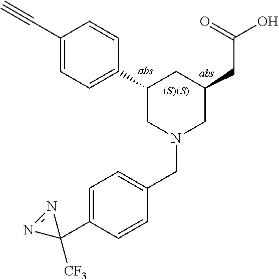

Anti-2-(5-(4-ethynylphenyl)-1-(4-(3-(trifluoromethyl)-3H-diazirin-3-yl)benzyl)piperidin-3-yl)acetic acid (40 mg) was further purified by Chiral SFC to give the title compound as fraction 2. Yield: 20% (8.0 mg, white solid). Chiral SFC (Method D) Rt. 1.283 min, 49.76% (Max) and Rt. 1.565 min, 50.24% (Max). ¹H NMR (400 MHz, DMSO-d₆) δ 12.10 (br.s, 1H), 7.44 (d, J=8.0 Hz, 2H), 7.38 (d, J=8.0 Hz, 2H), 7.32 (d, J=8.0 Hz, 2H), 7.22 (d, J=8.0 Hz, 2H), 4.12 (s, 1H), 3.55 (d, J=14.0 Hz, 1H), 3.46 (d, J=14.0 Hz, 1H), 3.03-2.92 (m, 1H), 2.69-2.67 (m, 1H), 2.53-2.29 (m, 6H), 1.71-1.60 (m, 2H). LCMS: (Method F) 442.2 (M+H), Rt. 1.38 min, 100% (Max). HPLC: (Method C) Rt. 5.72 min, 99.98% (Max). Chiral SFC: (Method D) (Rt: 1.642 min, EE: 100%).

### 137

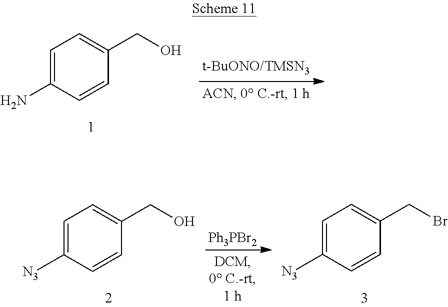 [SMILES: Nc1ccc(CO)cc1 ; [N-]=[N+]=Nc1ccc(CO)cc1 ; "Ph3PBr2" ; [N-]=[N+]=Nc1ccc(CBr)cc1]

### Step): (4-azidophenyl)methanol

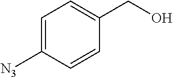 [SMILES: [N-]=[N+]=Nc1ccc(CO)cc1]

To a solution of (4-aminophenyl)methanol (1.00 g, 3.70 mmol) in MeCN (15 mL) was added tert-butyl nitrate (1.36 g, 5.50 mmol) drop-wise at 0° C. and followed by azidotrimethylsilane (1.50 g, 5.50 mmol). The resultant mixture was stirred at room temperature for 1 h. After completion (monitored by TLC), the mixture was diluted with water (20 mL) and extracted with EtOAc (15 mL×2). The combined organic layer was dried, and concentrated to give the title compound (crude, 1.32 g, yellow oil), which was used for next step without further purification.

### Step 2: 1-azido-4-(bromomethyl)benzene

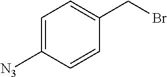 [SMILES: [N-]=[N+]=Nc1ccc(CBr)cc1]

To a solution of (4-azidophenyl)methanol (crude, 1.20 g, 8.10 mmol) in DCM (15 mL) was added dibromotriphenyl-15-phosphane (4.20 g, 9.60 mmol) in portions at 0° C. under N₂. The resultant mixture was stirred at room temperature for 1 h. After completion (monitored by TLC), the mixture was diluted with water (20 mL) and extracted with EtOAc (15 mL×2). The combined organic layer was dried, concentrated, and purified by silica gel column chromatography (PE/EA=100/1-50:1) to give the title compound. Yield: 53% (800 mg, yellow liquid). ¹H NMR (400 MHz, CDCl₃) δ 7.37 (d, J=8.0 Hz, 2H), 7.06-6.86 (d, J=8.0 Hz, 2H), 4.48 (s, 2H).

### Step 3: anti-2-(1-(4-azidobenzyl)-5-(4-ethynylphenyl)piperidin-3-yl)acetic acid

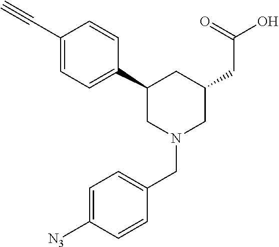 [SMILES: C#Cc1ccc([C@H]2C[C@H](CC(=O)O)CN(Cc3ccc(N=[N+]=[N-])cc3)C2)cc1]

Anti-2-(1-(4-azidobenzyl)-5-(4-ethynylphenyl)piperidin-3-yl)acetic acid was synthesized following the scheme 10, using 1-azido-4-(bromomethyl)benzene in step 9, without purification via chiral SFC in step 12. ¹H NMR (400 MHz, DMSO-d₆) δ 7.39-7.31 (m, 6H), 7.06 (d, J=8.0 Hz, 2H), 4.10 (s, 1H), 3.49 (d, J=13.6 Hz, 1H), 3.41 (d, J=13.6 Hz, 1H), 2.97 (s, 1H), 2.69-2.63 (m, 1H), 2.44-2.11 (m, 6H), 1.74-1.55 (m, 2H). LCMS: (Method F) 375.2 (M+H), Rt. 1.44 min, 100% (Max). HPLC: (Method C) Rt. 4.77 min, 97.57% (Max).

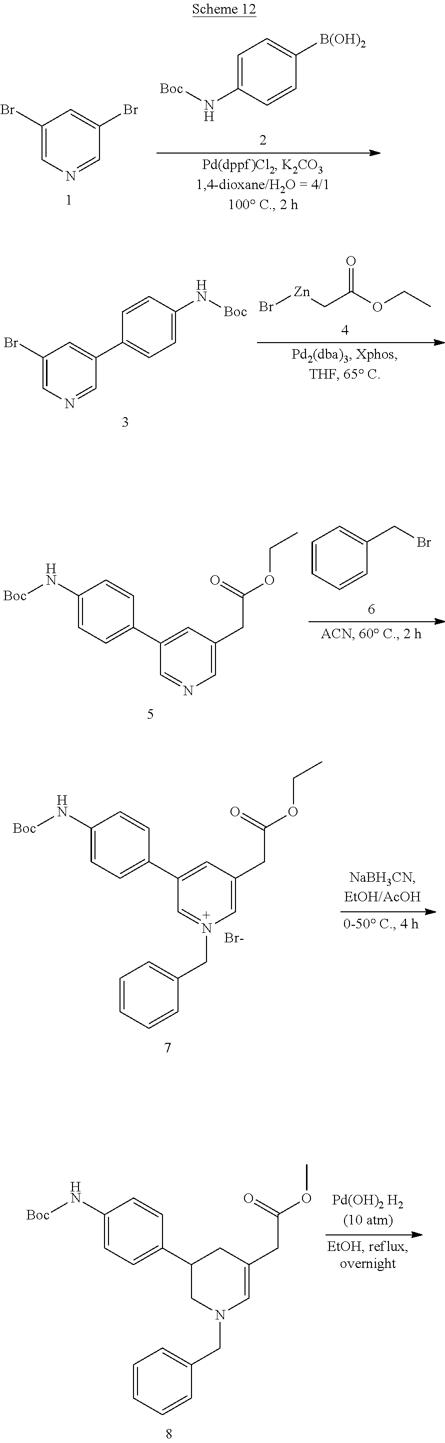

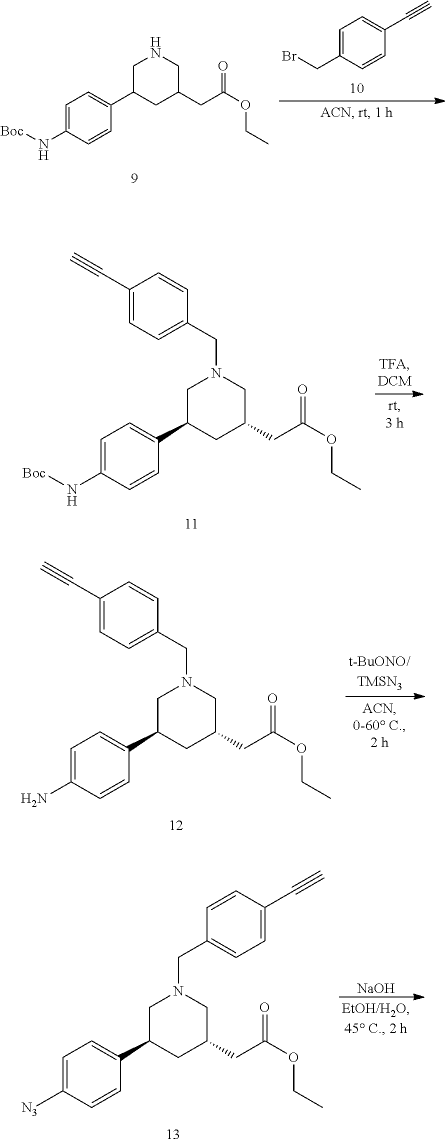

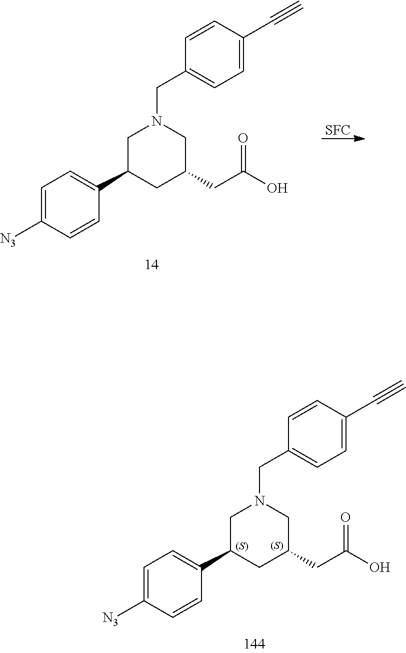

### Step 1: tert-butyl (4-(5-bromopyridin-3 yl)phenyl)carbamate

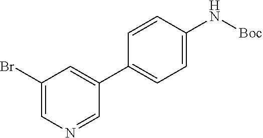 [SMILES: CC(C)(C)OC(=O)Nc1ccc(-c2cncc(Br)c2)cc1]

3,5-Dibromopyridine (10.0 g, 42.2 mmol), (4-((tert-butoxycarbonyl)amino)phenyl)boronic acid (6.70 g, 28.2 mmol), Pd(dppf)₂Cl₂·CH₂Cl₂ (2.30 g, 2.82 mmol), K₂CO₃ (11.7 g, 84.4 mmol), 1,4-dioxane (40 mL) and water (10 mL) were added to a 100 mL reaction flask. The mixture was degassed and purged with N₂ for 3 times. The mixture was stirred at 100° C. for 2 h. The mixture was filtered and the solid was washed with EtOAc. The filtrate was extracted with EtOAc for 3 times. The combined organic layer was dried, concentrated, and purified by silica gel chromatography (PE:EA=3:2) to give the title compound. Yield: 72% (7.10 g, white solid). LCMS: (Method G) 349.0 (M+H).

### Step 2: ethyl 2-(5-(4-((tert-butoxycarbonyl)amino)phenyl)pyridin-3-yl)acetate

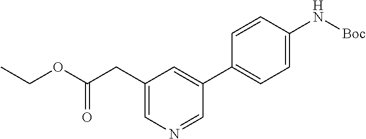 [SMILES: CCOC(=O)Cc1cncc(-c2ccc(NC(=O)OC(C)(C)C)cc2)c1]

tert-Butyl (4-(5-bromopyridin-3-yl)phenyl)carbamate (6.20 g, 17.8 mmol), (2-ethoxy-2-oxoethyl)zinc(II) bromide (1 mol/L in THF, 89 mL), Pd₂(dba)₃ (815 mg, 0.890 mmol), and Xphos (850 mg, 1.78 mmol) were added to a 250 mL flask. The mixture was stirred at 65° C. for 2 h under N₂. The mixture was quenched with sat.aq. NH₄Cl, and then filtered. The filtrate was extracted with EtOAc for 3 times. The combined organic layer was dried, concentrated, and purified by silica gel chromatography (PE:EA=2:1) to give the title compound. Yield: 52% (3.30 g, white solid). LCMS: (Method G) 357.2 (M+H).

### Step 3: 1-benzyl-3-(4-((tert-butoxycarbonyl)amino)phenyl)-5-(2-ethoxy-2-oxoethyl)pyridin-1-ium bromide

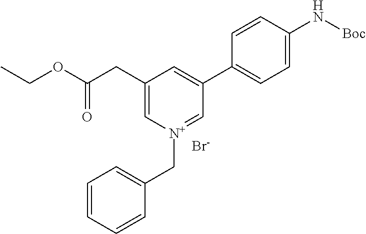 [SMILES: CCOC(=O)Cc1cc(-c2ccc(NC(=O)OC(C)(C)C)cc2)c[n+](Cc2ccccc2)c1 ; [Br-]]

Ethyl 2-(5-(4-((tert-butoxycarbonyl)amino)phenyl)pyridin-3-yl)acetate (4.20 g, 11.8 mmol), benzyl bromide (3.03 g, 17.7 mmol) and acetonitrile (42 mL) were added to a 100 mL flask. The mixture was stirred at 80° C. for 2 h. The mixture was concentrated under reduced pressure. The solid was washed with PE and dried at 50′C in vacuum to give the title compound as a yellow solid. Yield: crude (4.70 g, yellow solid).

### Step 4: ethyl 2-(1-benzyl-5-(4-((tert-butoxycarbonyl)amino)phenyl)-1,4,5,6-tetrahydropyridin-3-yl)acetate

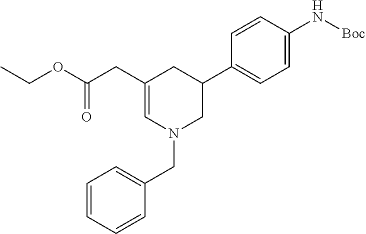 [SMILES: CCOC(=O)CC1=CN(Cc2ccccc2)CC(c2ccc(NC(=O)OC(C)(C)C)cc2)C1]

1-Benzyl-3-(4-((tert-butoxycarbonyl)amino)phenyl)-5-(2-ethoxy-2-oxoethyl)pyridin-1-ium bromide (4.60 g, 8.80 mmol) was dissolved in AcOH (23 mL) and EtOH (23 mL). NaBH₃CN (4.45 g, 70.8 mmol) was added slowly to the reaction mixture at 0° C. The mixture was stirred at 50° C. for 4 h. The reaction was quenched with Na₂CO₃ (aq) at 0° C. The mixture was extracted with EtOAc for 3 times. The organic layer was dried and concentrated under reduced pressure. The residue was used directly in next step without further purification. Yield: 90% (3.60 g, yellow oil). LCMS: (Method G) 451.2 (M+H).

### Step 5: ethyl 2-(5-(4-((tert-butoxycarbonyl)amino)phenyl)piperidin-3-yl)acetate

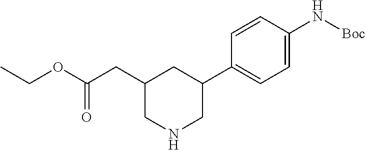 [SMILES: CCOC(=O)CC1CNCC(c2ccc(NC(=O)OC(C)(C)C)cc2)C1]

Ethyl 2-(1-benzyl-5-(4-((tert-butoxycarbonyl)amino)phenyl)-1,4,5,6-tetrahydropyridin-3-yl)acetate (3.60 g, 8.00 mmol), Pd/C (3.4 g, 10% (w %)) and EtOH (36 mL) were added to a 50 mL flask. The mixture was refluxed overnight under H₂ (10 atm). The mixture was filtered and the solid was washed with EtOH. The filtrate was concentrated under reduced pressure. The residue was purified by silica gel chromatography (DCM:MeOH=10:1) to give the title compound Yield: 78% (2.25 g, yellow oil). LCMS: (Method G) 363.2 (M+H).

### Step 6: anti-ethyl 2-(5-(4-((tert-butoxycarbonyl)amino)phenyl)-1-(4-ethynylbenzyl)piperidin-3-yl)acetate

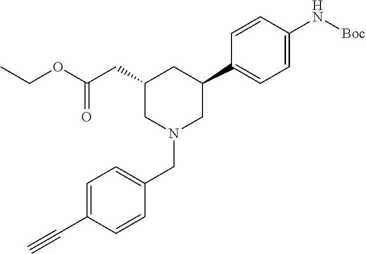 [SMILES: C#Cc1ccc(CN2C[C@H](CC(=O)OCC)C[C@@H](c3ccc(NC(=O)OC(C)(C)C)cc3)C2)cc1]

Ethyl 2-(5-(4-((tert-butoxycarbonyl)amino)phenyl)piperidin-3-yl)acetate (750 mg, 2.10 mmol) and DIPEA (178 mg, 1.38 mmol) were dissolved in MeCN (2 mL). 1-(Bromomethyl)-4-ethynylbenzene (323 mg, 1.66 mmol) in MeCN (2 mL) was added dropwise. The mixture was stirred at room temperature for 1 h. The mixture was diluted with water and extracted with EtOAc for 3 times. The combined organic layer was concentrated under reduced pressure. The residue was purified by silica gel chromatography (PE:EA=10:1) to give the title compound. Non-polar isomer (minor isomer, assigned as anti). Yield: 16% (163 mg, colorless oil). LCMS: (Method G) 477.3 (M+H).

### Step 7: anti-ethyl 2-(5-(4-aminophenyl)-1-(4-ethynylbenzyl)piperidin-3-yl)acetate

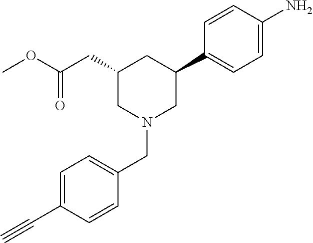 [SMILES: C#Cc1ccc(CN2C[C@H](CC(=O)OC)C[C@@H](c3ccc(N)cc3)C2)cc1]

Anti-ethyl 2-(5-(4-((tert-butoxycarbonyl)amino)phenyl)-1-(4-ethynylbenzyl) piperidin-3-yl)acetate (214 mg, 0.45 mmol) was dissolved in DCM. TFA (1.28 g, 11.2 mmol) was added to the mixture at room temperature. The mixture was stirred at room temperature for 3 h. The mixture was neutralized with sat. aq. NaHCO₃. The mixture was extracted with DCM. The organic layer was dried and concentrated under reduced pressure. The residue was used directly in next step without further purification. Yield: crude (160 mg, light yellow oil).

### Step 8: anti-ethyl 2-(5-(4-azidophenyl)-1-(4-ethynylbenzyl)piperidin-3-yl)acetate

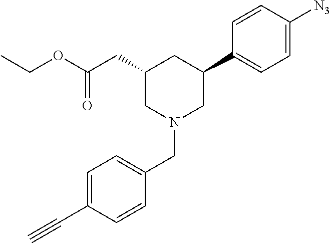 [SMILES: C#Cc1ccc(CN2C[C@H](CC(=O)OCC)C[C@@H](c3ccc(N=[N+]=[N-])cc3)C2)cc1]

tert-Butyl nitrite (103 mg, 1.00 mmol) and azidotrimethylsilane (118 mg, 1.00 mmol) were added to a solution of anti-ethyl 2-(5-(4-aminophenyl)-1-(4-ethynylbenzyl)piperidin-3-yl)acetate (160 mg, 0.43 mmol) in MeCN at OC in order. The mixture was then stirred at 60° C. for 2 h. The mixture was cooled to room temperature and diluted with water. The mixture was extracted with EtOAc for 3 times. The organic layer was concentrated, and purified by silica gel chromatography (PE:EA=20:1) to give the title compound. Yield: 80% (137 mg, colorless oil). LCMS: (Method G) 403.2 (M+H).

### Step 9: anti-2-(5-(4-(azido)phenyl)-1-(4-ethynylbenzyl)piperidin-3-yl)acetic acid

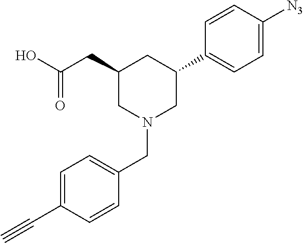 [SMILES: C#Cc1ccc(CN2C[C@@H](CC(=O)O)C[C@H](c3ccc(N=[N+]=[N-])cc3)C2)cc1]

To a solution of anti-ethyl 2-(5-(4-azidophenyl)-1-(4-ethynylbenzyl)piperidin-3-yl)acetate (137 mg, 0.34 mmol) in EtOH/water (3:1, 4 mL) was added NaOH (40.8 mg, 1.02 mmol) at room temperature. The mixture was stirred at 40° C. for 2 h. After completion (the reaction mixture was monitored by TLC), the organic solvent was removed under vacuum. The residue was acidified with aq. HCl (3 N) to pH 4˜5. The precipitated solid was filtered and purified to give the title compound. Prep-HPLC: (Method E). Yield: 65% (90 mg, white solid). LCMS: (Method G) 375.2 (M+H)

### Step 10: 2-((3S,5S)-5-(4-(azido)phenyl)-1-(4-ethynylbenzyl)piperidin-3-yl)acetic acid

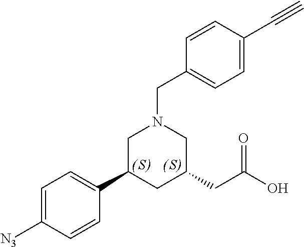

Anti-2-(5-(4-(azido)phenyl)-1-(4-ethynylbenzyl)piperidin-3-yl)acetic acid (90 mg) was further purified by Chiral SFC to give the title compound as fraction 1. Chiral SFC (Method G) Rt. 1.045 min, 48.48% (Max) and Rt. 1.363 min, 48.31% (Max). Yield: 18% (16.2 mg, white solid). ¹H NMR (400 MHz, DMSO-d₆) δ 11.74 (br.s, 1H), 7.42 (d, J=8.0 Hz, 2H), 7.35 (d, J=8.4 Hz, 1H), 7.31 (d, J=8.0 Hz, 4H), 7.05-6.99 (m, 2H), 4.13 (s, 1H), 3.52 (d, J=13.6 Hz, 1H), 3.43 (d, J=13.6 Hz, 1H), 3.00-2.92 (m, 1H), 2.73-2.66 (m, 1H), 2.48-2.07 (m, 6H), 1.73-1.60 (m, 2H). LCMS: (Method F) 375.2 (M+H), Rt. 1.35 min, 91.42% (Max). HPLC: (Method D) Rt. 6.70 min, 96.48% (Max). Chiral SFC: (Method G) (Rt: 1.047 min, EE: 100%).

### 145

### 2-((3S,5S)-1-(4-(azidomethyl)benzyl)-5-(4-azidophenyl)piperidin-3-yl)acetic acid

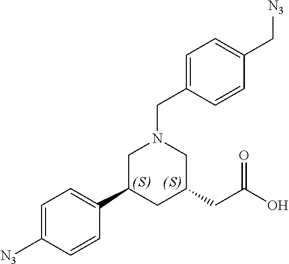

145 was synthesized following the route of scheme 12, substituting 1-(azidomethyl)-4-(bromomethyl)benzene in step 10. Isomers were separated by Chiral SFC to give the title compound as fraction 2. Chiral SFC: (method E) Rt. 1.516 min, 49.09% (Max) and Rt. 2.216 min, 48.47% (Max). ¹H NMR (400 MHz, CD₃OD): δ 7.46 (d, J=8.0 Hz, 2H), 7.38 (d, J=8.0 Hz, 2H), 7.30 (d, J=8.0 Hz, 2H), 7.01-6.98 (m, 2H), 4.38 (s, 2H), 3.90 (dd, J=15.6, 13.6 Hz, 2H), 3.22-3.16 (m, 1H), 3.08 (d, J=8.0 Hz, H), 2.79 (dd, J=12.0, 4.0 Hz, 1H), 2.65 (t, J=8.0 Hz, 2H), 2.57 (d, J=8.0 Hz, 2H), 2.39 (s, 1H), 1.96-1.83 (m, 2H). LCMS: (Method F) 406.2 (M+H), Rt. 1.36 min, 100% (Max), HPLC: (Method D) Rt. 6.62 min, 98.93% (Max). Chiral SFC: (Method E) Rt. 2.198 min, 100% (Max).

### 129

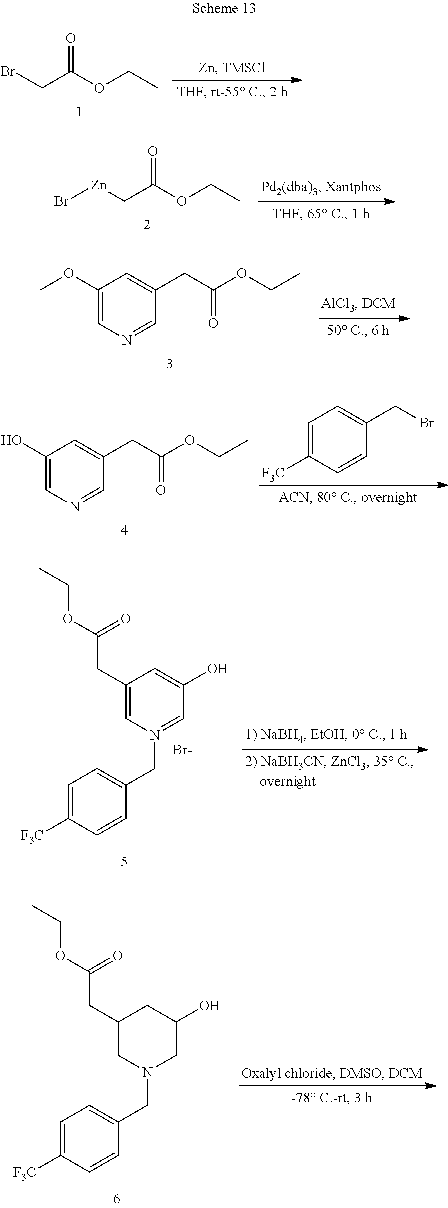

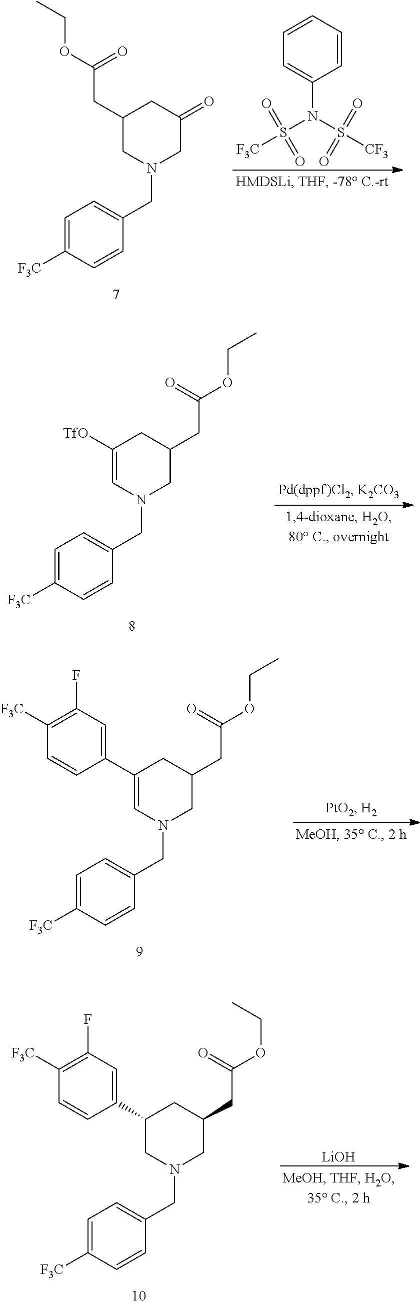 [SMILES: CCOC(=O)CC1CC(=O)CN(Cc2ccc(C(F)(F)F)cc2)C1 ; O=S(=O)(N(c1ccccc1)S(=O)(=O)C(F)(F)F)C(F)(F)F ; CCOC(=O)CC1CC(OS(=O)(=O)C(F)(F)F)=CN(Cc2ccc(C(F)(F)F)cc2)C1 ; CCOC(=O)CC1CC(c2ccc(C(F)(F)F)c(F)c2)=CN(Cc2ccc(C(F)(F)F)cc2)C1 ; CCOC(=O)C[C@@H]1C[C@@H](c2ccc(C(F)(F)F)c(F)c2)CN(Cc2ccc(C(F)(F)F)cc2)C1 ; "LiOH"]

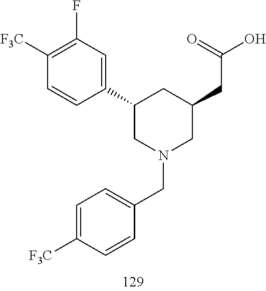 [SMILES: O=C(O)C[C@@H]1C[C@@H](c2ccc(C(F)(F)F)c(F)c2)CN(Cc2ccc(C(F)(F)F)cc2)C1]

### Step 1: (2-ethoxy-2-oxoethyl)zinc(II) bromide

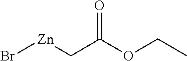

To a solution of active Zinc powder (27.6 g, 425 mmol) in anhydrous tetrahydrofuran (150 mL) was added chlorotrimethylsilane (2.70 mL, 21.2 mmol). The reaction was stirred at 50° C. for 1.5 h under nitrogen atmosphere. Then ethyl bromoacetate (23.6 mL, 212 mmol) in anhydrous tetrahydrofuran (150 mL) was added dropwise and the reaction was stirred at 50° C. for 30 mins. The light green liquid was used directly into the next step.

### Step 2: ethyl 2-(5-methoxypyridin-3-yl)acetate

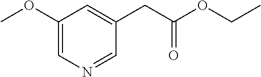

To a solution of 3-bromo-5-methoxypyridine (20.0 g, 106 mmol) in anhydrous tetrahydrofuran (50 mL) was added 4,5-bis(diphenylphosphino)-9,9-dimethylxanthene (3.08 g, 5.32 mmol), tris(dibenzylideneacetone)dipalladium (2.44 g, 2.66 mmol) under nitrogen atmosphere. Then the solution of (2-ethoxy-2-oxoethyl)zinc(II) bromide from step 1 was added to the reaction and the reaction was stirred at 65° C. for 1 h. When the reaction was completed, the reaction was concentrated. The residue was purified by flash column chromatography (petroleum ether/ethyl acetate=10/1) to give the title compound. Yield: 79% (18.0 g, orange oil). LCMS: (Method H) 196.2 (M+H).

### Step 3: ethyl 2-(5-hydroxypyridin-3-yl)acetate

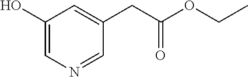

To a solution of ethyl 2-(5-methoxypyridin-3-yl)acetate (10.0 g, 51.3 mmol) in dichloromethane (60 mL) was slowly added aluminum chloride (34.2 g, 256 mmol) at 0° C. The mixture was warmed to 50° C. then stirred for 6 h at that temperature. Then the mixture was cooled to room temperature, filtered, and the filtrate was concentrated. The residue was purified by flash column chromatography (methanol/dichloromethane=1/10) to give the title compound. Yield: 59% (5.50 g, colorless oil). LCMS: (Method H) 182.2 (M+H).

### Step 4: 3-(2-ethoxy-2-oxoethyl)-5-hydroxy-1-(4-(trifluoromethyl)benzyl)pyridine-1-ium

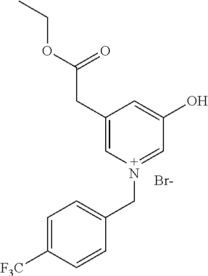 [SMILES: CCOC(=O)Cc1cc(O)c[n+](Cc2ccc(C(F)(F)F)cc2)c1 ; [Br-]]

To a solution of ethyl 2-(5-hydroxypyridin-3-yl)acetate (3.00 g, 16.6 mmol) in acetonitrile (10 mL) was added 1-(bromomethyl)-4-(trifluoromethyl)benzene (3.96 g, 16.6 mmol). The mixture was stirred at 80° C. overnight. The resulting mixture was concentrated. The residue was triturated with dichloromethane to give the title compound. Yield: 53% (3.00 g, white solid). LCMS: (Method H) 340.1 (M+H).

### Step 5: ethyl 2-(5-hydroxy-1-(4-(trifluoromethyl)benzyl)piperidin-3-yl)acetate

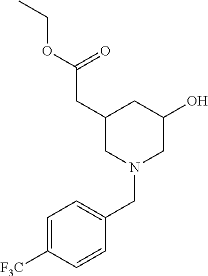 [SMILES: CCOC(=O)CC1CC(O)CN(Cc2ccc(C(F)(F)F)cc2)C1]

To a solution of 3-(2-ethoxy-2-oxoethyl)-5-hydroxy-1-(4-(trifluoromethyl)benzyl)pyridine-1-ium (3.00 g, 8.82 mmol) in ethanol (10 mL) was added sodium borohydride (1.11 g, 17.6 mmol) at 0° C. The reaction was stirred at 0° C. for 1 h. Then the solvent was concentrated and water (30 mL) was added. The mixture was extracted by dichloromethane (10 mL×3). The organic layer was dried over anhydrous sodium sulfate, filtered, and concentrated. The residue was dissolved in methanol (10 mL), and then anhydrous Zinc chloride (1.20 g, 8.82 mmol) and sodium cyanoborohydride (1.66 g, 26.4 mmol) were added. The resulting mixture was stirred at 35° C. overnight. Then the mixture was poured into water and extracted by dichloromethane (10 mL×3). The organic layer was dried over sodium sulfate, filtered, and concentrated. The residue was purified by flash column chromatography (methanol/dichloromethane=1/10) to give the title compound. Yield: 49% (1.50 g, colorless oil). LCMS: (Method H) 346.1 (M+H)⁺.

### Step 6: ethyl 2-(5-oxo-1-(4-(trifluoromethyl)benzyl)piperidin-3-yl)acetate

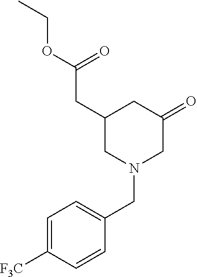 [SMILES: CCOC(=O)CC1CC(=O)CN(Cc2ccc(C(F)(F)F)cc2)C1]

To a solution of oxalyl chloride (387 mg, 3.05 mmol) in dichloromethane (10 mL) was added dimethyl sulfoxide (397 mg, 5.01 mmol) at −78° C. After stirring for 15 mins, a solution of ethyl 2-(5-hydroxy-1-(4-(trifluoromethyl)benzyl)piperidin-3-yl)acetate (700 mg, 2.04 mmol) in dichloromethane (2 mL) was added. The resulting reaction mixture was stirred at −78° C. for 15 mins, and then triethylamine (1.03 g, 10.2 mmol) was added. The reaction mixture was stirred at −78° C. for 30 mins and slowly warmed to room temperature for 2 h. Then the mixture was diluted with dichloromethane and washed with aqueous saturated NaHCO₃ solution and brine. The organic layer was dried over anhydrous sodium sulfate, filtered, and concentrated. The residue was purified by column chromatography (petroleum ether/ethyl acetate=1/4) to give the title compound. Yield: 57% (400 mg, yellow oil). LCMS: (Method H) 344.2 (M+H).

### Step 7: ethyl 2-(1-(4-(trifluoromethyl)benzyl)-5-(((trifluoromethyl)sulfonyl)oxy)-1,2,3,4-tetrahydropyridin-3-yl)acetate

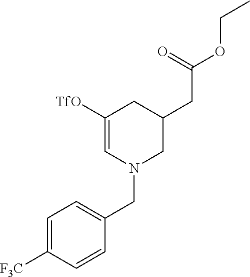 [SMILES: CCOC(=O)CC1CC(OS(=O)(=O)C(F)(F)F)=CN(Cc2ccc(C(F)(F)F)cc2)C1]

To a solution of ethyl 2-(5-oxo-1-(4-(trifluoromethyl)benzyl)piperidin-3-yl)acetate (150 mg, 0.437 mmol) in anhydrous tetrahydrofuran (2 mL) was added [bis(trimethylsilyl)amino] lithium (0.48 mL, 0.480 mmol) (1 mol/L in tetrahydrofuran) dropwise during 30 mins at −78° C. After stirring for 30 mins, a solution of 1,1,1-trifluoro-N-phenyl-N-((trifluoromethyl)sulfonyl)methanesulfonamide (164 mg, 0.460 mmol) in tetrahydrofuran (0.5 mL) was added. The resulting mixture was stirred for another 10 mins at −78° C. and slowly warmed to 0° C. After completion, the reaction was quenched with saturated sodium bicarbonate (1 mL) and extracted by dichloromethane (3 mL×2). The organic layer was dried by anhydrous sodium sulfate, filtered and concentrated. The residue was purified by column chromatography (petroleum ether/ethyl acetate=5/1) to give the title compound. Yield: 48% (100 mg, orange oil). LCMS: (Method H) 476.2 (M+H).

### Step 8: ethyl 2-(5-(3-fluoro-4-(trifluoromethyl)phenyl)-1-(4-(trifluoromethyl)benzyl)-1,2,3,4-tetrahydropyridin-3-yl)acetate

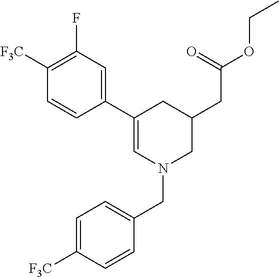 [SMILES: CCOC(=O)CC1CC(c2ccc(C(F)(F)F)c(F)c2)=CN(Cc2ccc(C(F)(F)F)cc2)C1]

To a solution of ethyl 2-(1-(4-(trifluoromethyl)benzyl)-5-(((trifluoromethyl)sulfonyl)oxy)-1,2,3,4-tetrahydropyridin-3-yl)acetate (370 mg, 0.780 mmol), (3-fluoro-4-(trifluoromethyl)phenyl)boronic acid (178 mg, 0.860 mmol) and potassium carbonate (323 mg, 2.34 mmol) in 1,4-dioxane (4 mL) and water (1 mL) was added 1,1′-bis(diphenylphosphino)ferrocene palladium(II) dichloride (57.0 mg, 0.078 mmol). The reaction mixture was degassed and refilled with N₂ 3 times. The mixture was stirred at 80° C. overnight. The resulting mixture was poured into water and extracted by ethyl acetate (10 mL×3). The organic layer was dried by anhydrous sodium sulfate, filtered, and concentrated. The residue was purified by column chromatography (petroleum ether/ethyl acetate=3/1) to give the title compound. Yield: 79% (260 mg, yellow oil). LCMS: (Method H) 490.2 (M+H).

### Step 9: anti-ethyl 2-(5-(3-fluoro-4-(trifluoromethyl)phenyl)-1-(4-(trifluoromethyl)benzyl)piperidin-3-yl)acetate

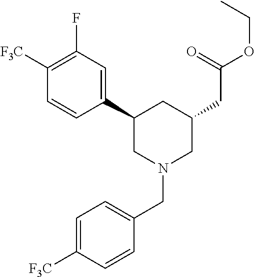 [SMILES: CCOC(=O)C[C@H]1C[C@H](c2ccc(C(F)(F)F)c(F)c2)CN(Cc2ccc(C(F)(F)F)cc2)C1]

To a solution of ethyl 2-(5-(3-fluoro-4-(trifluoromethyl)phenyl)-1-(4-(trifluoromethyl)benzyl)-1,2,3,4-tetrahydropyridin-3-yl)acetate (350 mg, 0.710 mmol) in methanol (5 mL) was added platinum dioxide (50 mg). The mixture was stirred at 35° C. for 2 h under hydrogen atmosphere. After completion, the mixture was filtered. The filtrate was concentrated. The residue was purified by column chromatography (petroleum ether/ethyl acetate=3/1) to give the title compound. Yield: 12% (41 mg, colorless oil). LCMS: (Method H) 492.2 (M+H).

### Step 10: anti-2-(5-(3-fluoro-4-(trifluoromethyl)phenyl)-1-(4-(trifluoromethyl)benzyl)piperidin-3-yl)acetic acid

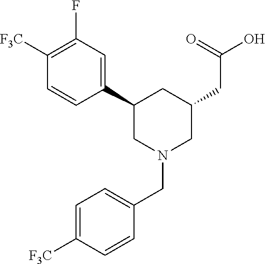 [SMILES: O=C(O)C[C@H]1C[C@H](c2ccc(C(F)(F)F)c(F)c2)CN(Cc2ccc(C(F)(F)F)cc2)C1]

To a solution of anti-ethyl 2-(5-(3-fluoro-4-(trifluoromethyl)phenyl)-1-(4-(trifluoromethyl)benzyl)piperidin-3-yl)acetate (41.0 mg, 0.0830 mmol) in tetrahydrofuran (0.6 mL), water (0.2 mL) and methanol (0.2 mL) was added lithium hydroxide (32.0 mg, 1.35 mmol). The resulting mixture was stirred at 35° C. for 2 h. The mixture was adjusted to pH=4˜5 and concentrated. The residue was purified via prep-HPLC to give the title compound. Prep-HPLC (Method C). Yield: 39% (14.8 mg, off-white solid). ¹H NMR (400 MHz, DMSO-d₆) δ 7.69-7.67 (m, 3H), 7.55-7.51 (m, 3H), 7.41 (d, J=8.4 Hz, 1H), 3.64-3.53 (m, 2H), 3.13-3.11 (s, 1H), 2.71-2.68 (m, 1H), 2.43-2.32 (m, 5H), 1.79-1.78 (m, 1H), 1.78-1.76 (m, 1H), 1.66-1.62 (m, 1H). LCMS: (Method H) 464.1 (M+H), Rt. 0.99 min, 100.00% (Max). HPLC: (Method E) Rt. 3.62 min, 99.55% (Max).

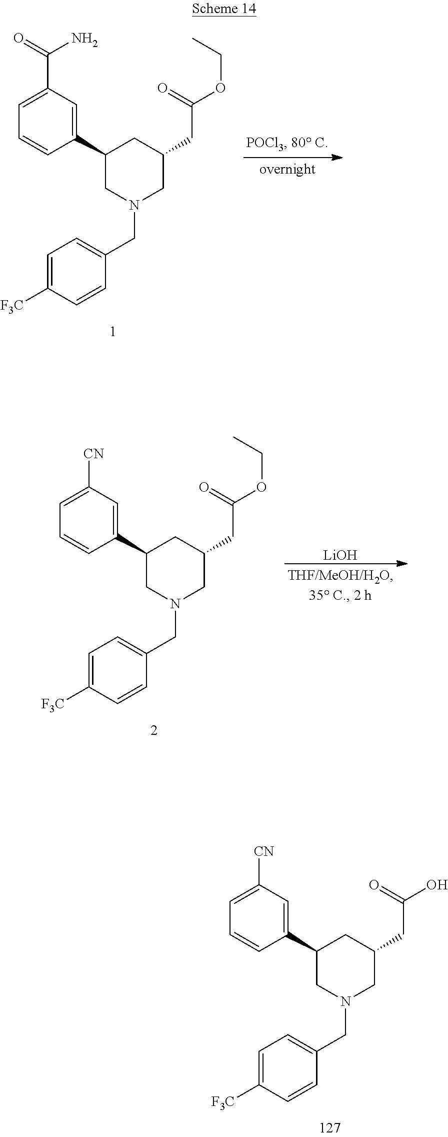 [SMILES: CCOC(=O)C[C@H]1C[C@H](c2cccc(C(N)=O)c2)CN(Cc2ccc(C(F)(F)F)cc2)C1 ; CCOC(=O)C[C@H]1C[C@H](c2cccc(C#N)c2)CN(Cc2ccc(C(F)(F)F)cc2)C1 ; "LiOH" ; N#Cc1cccc([C@H]2C[C@H](CC(=O)O)CN(Cc3ccc(C(F)(F)F)cc3)C2)c1]

### Step 1: anti-ethyl 2-(5-(3-cyanophenyl)-1-(4-(trifluoromethyl)benzyl)piperidin-3-yl)acetate

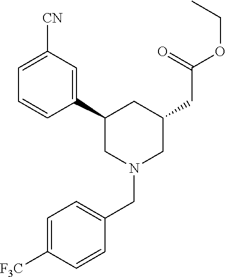 [SMILES: CCOC(=O)C[C@H]1C[C@H](c2cccc(C#N)c2)CN(Cc2ccc(C(F)(F)F)cc2)C1]

The solution of ethyl 2-(5-(3-carbamoylphenyl)-1-(4-(trifluoromethyl)benzyl)-1,2,3,4-tetrahydropyridin-3-yl)acetate (90 mg, 0.20 mmol, synthesized following the route of scheme 12, substituting (3-carbamoylphenyl)boronic acid in step 8) in phosphorus oxychloride (2 mL) was stirred at 80° C. overnight. The reaction mixture was concentrated, poured into ice-water and extracted by dichloromethane (10 mL×3). The organic layer was dried over anhydrous sodium sulfate, filtered, and concentrated. The residue was purified by column chromatography (methanol/dichloromethane=1/20) to give the title compound. Yield: 53% (46.0 mg, colorless oil). LCMS: (Method H) 431.2 (M+H).

### Step 2: anti-2-(5-(3-cyanophenyl)1-(4-(trifluoromethyl)benzyl)piperidin-3-yl)acetic acid

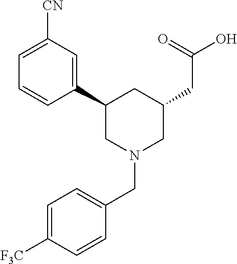 [SMILES: N#Cc1cccc([C@H]2C[C@H](CC(=O)O)CN(Cc3ccc(C(F)(F)F)cc3)C2)c1]

To a solution of anti-ethyl 2-(5-(3-cyanophenyl)-1-(4-(trifluoromethyl)benzyl)piperidin-3-yl)acetate (46.0 mg, 0.107 mmol) in tetrahydrofuran (0.6 mL), water (0.2 mL) and methanol (0.2 mL) was added lithium hydroxide (13.0 mg, 0.535 mmol). The resulting mixture was stirred at 35° C. for 2 h. The mixture was adjusted to pH=4˜5 and concentrated. The residue was purified via prep-HPLC to give the title compound. Prep-HPLC: (Method C). Yield: 50% (21.4 mg, off-white solid). ¹H NMR (400 MHz, DMSO-d₆) δ 12.09 (br,s, 1H), 7.85 (s, 1H), 7.68-7.67 (m, 4H), 7.57-7.50 (m, 3H), 3.67-3.53 (m, 2H), 3.10-3.07 (m, 1H), 2.75-2.70 (m, 1H), 2.47-2.35 (m, 5H), 1.85 (s, 1H), 1.79-1.78 (m, 1H), 1.68-1.63 (m, 1H). LCMS: (Method H) 403.2 (M+H), Rt. 0.90 min, 100.00% (Max). HPLC: (Method E) Rt. 2.53 min, 98.77% (Max).

### 134

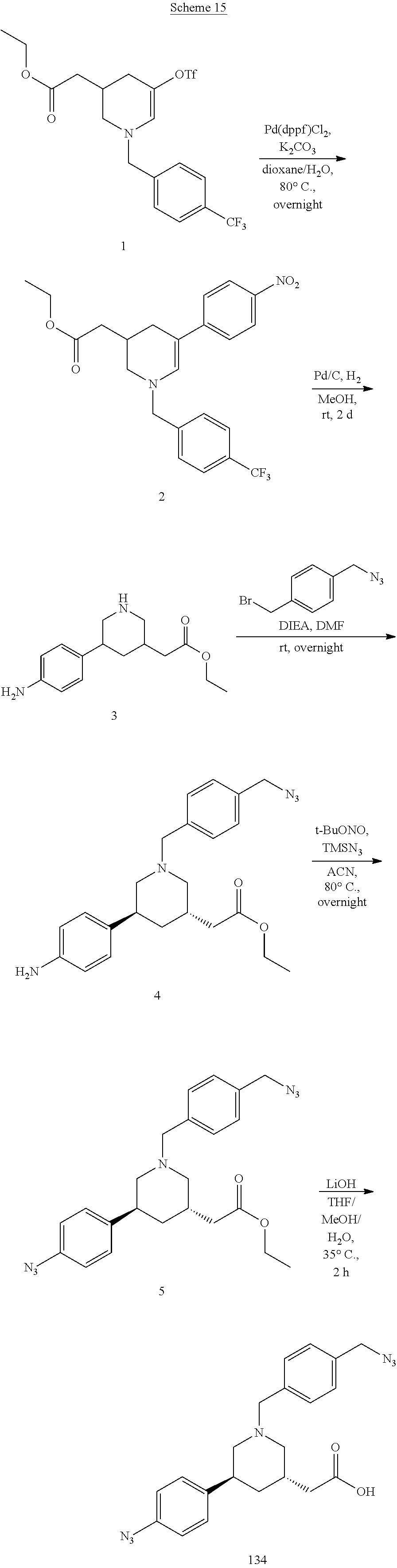

### Step 1: ethyl 2-(5-(4-nitrophenyl)-1-(4-(trifluoromethyl)benzyl)-1,2,3,4-tetrahydropyridin-3-yl)acetate

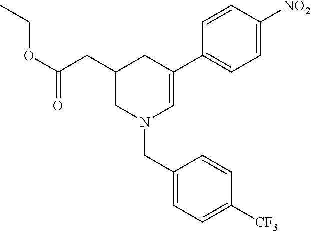 [SMILES: CCOC(=O)CC1CC(c2ccc([N+](=O)[O-])cc2)=CN(Cc2ccc(C(F)(F)F)cc2)C1]

To a solution of ethyl 2-(1-(4-(trifluoromethyl)benzyl)-5-(((trifluoromethyl)sulfonyl)oxy)-1,2,3,4-tetrahydropyridin-3-yl)acetate (320 mg, 0.460 mmol), (4-nitrophenyl)boronic acid (85.0 mg, 0.510 mmol) and potassium carbonate (191 mg, 1.39 mmol) in 1,4-dioxane (4 mL) and water (1 mL) was added 1,1′-bis(diphenylphosphino)ferrocene palladium(II) dichloride (34.0 mg, 0.046 mmol). The mixture was degassed and refilled with N₂ for 3 times. The reaction was stirred at 80° C. overnight under nitrogen atmosphere. The resulting mixture was poured into water and extracted with ethyl acetate (10 mL×3). The organic layer was dried by anhydrous sodium sulfate, filtered, and concentrated. The residue was purified by column chromatography (petroleum ether/ethyl acetate=2/1) to give the title compound. Yield: 73% (220 mg, yellow solid). LCMS: (Method H) 449.2 (M+H).

### Step 2: ethyl 2-(5-(4-aminophenyl)piperidin-3-yl)acetate

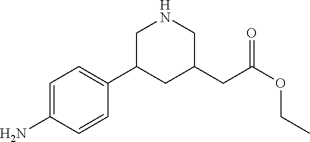 [SMILES: CCOC(=O)CC1CNCC(c2ccc(N)cc2)C1]

To a solution of ethyl 2-(5-(4-nitrophenyl)-1-(4-(trifluoromethyl)benzyl)-1,2,3,4-tetrahydropyridin-3-yl)acetate (220 mg, 0.490 mmol) in methanol (3 mL) was added 10% Palladium on activated carbon (22 mg). The mixture was stirred at room temperature for 48 hours under hydrogen atmosphere. When the reaction was completed, the resulting mixture was filtered and the filtrate was concentrated to give the title compound. Yield: crude (130 mg, brown oil). LCMS: (Method H) 263.2 (M+H).

### Step 3: anti-ethyl 2-(5-(4-aminophenyl)-1-(4-(azidomethyl)benzyl)piperidin-3-yl)acetate

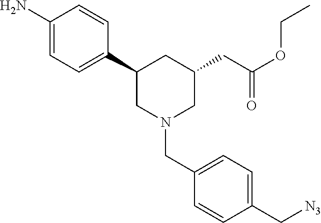 [SMILES: CCOC(=O)C[C@H]1C[C@H](c2ccc(N)cc2)CN(Cc2ccc(CN=[N+]=[N-])cc2)C1]

To a solution of ethyl 2-(5-(4-aminophenyl)piperidin-3-yl)acetate (130 mg, 0.500 mmol) and N,N-diisopropylethylamine (190 mg, 1.49 mmol) in acetonitrile (3 mL) was added 1-(azidomethyl)-4-(bromomethyl)benzene (135 mg, 0.595 mmol). The resulting mixture was stirred at room temperature overnight. The solvent was removed and the residue was purified by Prep-TLC (petroleum/ethyl acetate=2/1) to give the title compound. Yield: 15% (30 mg, red oil). LCMS: (Method H) 408.2 (M+H).

### Step 4: anti-ethyl 2-(1-(4-(azidomethyl)benzyl)-5-(4-azidophenyl)piperidin-3-yl)acetate

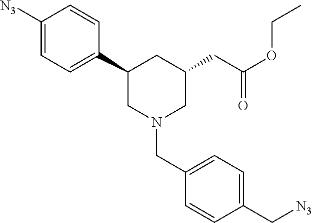 [SMILES: CCOC(=O)C[C@H]1C[C@H](c2ccc(N=[N+]=[N-])cc2)CN(Cc2ccc(CN=[N+]=[N-])cc2)C1]

A solution of anti-ethyl 2-(5-(4-aminophenyl)-1-(4-(azidomethyl)benzyl)piperidin-3-yl)acetate (70.0 mg, 0.170 mmol) in acetonitrile (3 mL) was cooled to 0° C., and tert-butyl nitrite (26.6 mg, 0.258 mmol) was added, followed by azidotrimethylsilane (29.7 mg, 0.258 mmol). The reaction mixture was stirred at 80° C. overnight. The mixture was concentrated, and the residue was purified by Prep-TLC (petroleum ether/ethyl acetate=3/1) to give the title compound. Yield: 56% (60 mg, red oil). LCMS: (Method H) 434.2 (M+H).

### Step 5: anti-2-(1-(4-(azidomethyl)benzyl)-5-(4-azidophenyl)piperidin-3-yl)acetic acid

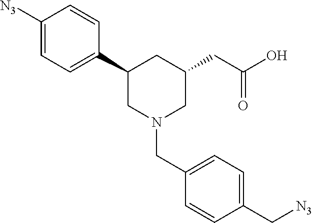 [SMILES: [N-]=[N+]=NCc1ccc(CN2C[C@@H](CC(=O)O)C[C@H](c3ccc(N=[N+]=[N-])cc3)C2)cc1]

To a solution of anti-ethyl 2-(1-(4-(azidomethyl)benzyl)-5-(4-azidophenyl)piperidin-3-yl)acetate (40.0 mg, 0.0920 mmol) in tetrahydrofuran (0.6 mL), methanol (0.2 mL) and water (0.2 mL) was added lithium hydroxide (11.0 mg, 0.460 mmol). The mixture was stirred at 35° C. for 2 h. The mixture was adjusted to pH=4˜5 and concentrated. The residue was purified via prep-HPLC to give the title compound. Prep-HPLC: (Method C). Yield: 29% (11 mg, off-white solid). 1H NMR (400 MHz, DMSO-d₆) δ 8.42 (br.s, 1H), 7.38-7.30 (m, 6H), 7.03 (d, J=8.4 Hz, 2H), 4.44 (s, 2H), 3.54-3.46 (m, 2H), 2.68-2.66 (m, 2H), 2.47-2.45 (m, 2H), 2.42-2.17 (m, 5H), 1.71-1.62 (m, 1H). LCMS: (Method H) 406.3 (M+H), Rt. 1.28 min, 100.00% (Max). HPLC: (Method E) Rt. 2.41 min, 99.31% (Max).

### 132

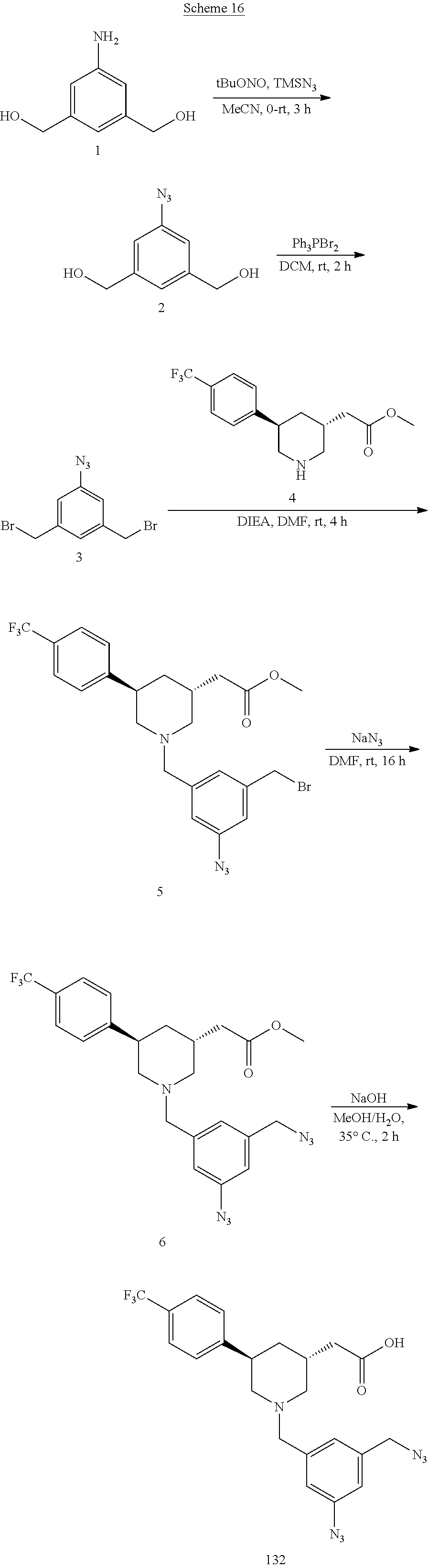

### Step 1: (5-azido-1,3-phenylene)dimethanol

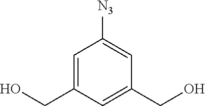 [SMILES: [N-]=[N+]=Nc1cc(CO)cc(CO)c1]

To a solution of (5-amino-1,3-phenylene)dimethanol (153 mg, 1.00 mmol) in MeCN (5 mL) was added t-BuONO (155 mg, 1.50 mmol) slowly at 0° C., followed by addition of TMSN₃ (138 mg, 1.20 mmol). The resulting mixture was stirred at room temperature for 3 h. Water (5 mL) was added to the reaction, and then the mixture was extracted with EtOAc (20 mL×2). The combined organic layers were washed with brine (20 mL), dried over anhydrous NaSO₄, and concentrated to afford the title compound. Yield: crude (170 mg, brown solid). ¹H NMR (400 MHz, CDCl₃) δ 7.14-7.13 (m, 1H), 6.98 (s, 2H), 4.70 (s, 4H).

### Step 2: 1-azido-3,5-bis(bromomethyl)benzene

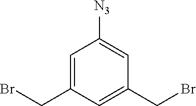 [SMILES: [N-]=[N+]=Nc1cc(CBr)cc(CBr)c1]

To a solution of (5-azido-1,3-phenylene)dimethanol (170 mg, 0.950 mmol) in DCM (5 mL) was added Ph₃PBr₂ (922 mg, 2.18 mmol) in portions. After stirred at room temperature for 2 h, the mixture was quenched with saturated aqueous Na₂S₂O₃ (20 mL), and extracted with DCM (20 mL×3). The combined organic layers were dried over anhydrous Na₂SO₄, concentrated, and purified by flash chromatography on silica gel (EtOAc/PE=1/20) to afford the title compound. Yield: 56% for 2 steps (170 mg, yellow solid). ¹H NMR (400 MHz, CDCl₃) δ 7.18 (t, J=2.0 Hz, 1H), 6.98 (d, J=2.0 Hz, 2H), 4.43 (s, 4H).

### Step 3: anti-methyl 2-(1-(3-azido-5-(bromomethyl)benzyl)-5-(4-(trifluoromethyl) phenyl)piperidin-3-yl)acetate

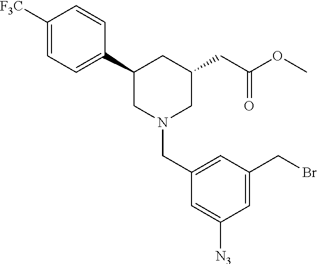 [SMILES: COC(=O)C[C@H]1C[C@H](c2ccc(C(F)(F)F)cc2)CN(Cc2cc(CBr)cc(N=[N+]=[N-])c2)C1]

To a solution of 1-azido-3,5-bis(bromomethyl)benzene (60.0 mg, 2.00 mmol) and anti-methyl 2-(5-(4-(trifluoromethyl)phenyl) piperidin-3-yl) acetate (61.0 mg, 0.200 mmol) in DMF (4 mL) was added DIPEA (400 uL, 2.4 mmol). After stirred at room temperature for 4 h, the mixture was diluted with water (20 mL), and extracted with EtOAc (20 mL×2). The combined organic layers were washed with brine (20 mL), dried over anhydrous NaSO₄ and concentrated. The residue was purified by flash chromatography on silica gel (EtOAc/PE=1/5) to afford the title compound. Yield: 38% (20.0 mg, yellow solid).

### Step 4: anti-methyl 2-(1-(3-azido-5-(azidomethyl)benzyl)-5-(4-(trifluoromethyl) phenyl)piperidin-3-yl)acetate

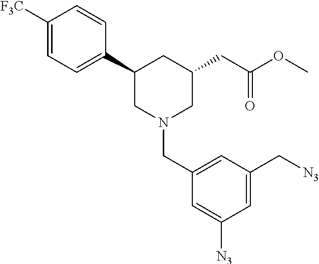 [SMILES: COC(=O)C[C@H]1C[C@H](c2ccc(C(F)(F)F)cc2)CN(Cc2cc(CN=[N+]=[N-])cc(N=[N+]=[N-])c2)C1]

To a solution of anti-methyl 2-(1-(3-azido-5-(bromomethyl) benzyl)-5-(4-(trifluoromethyl) phenyl) piperidin-3-yl)acetate (20.0 mg, 0.0400 mmol) in DMF (2 mL) was added NaN₃ (5.20 mg, 0.0800 mmol). After stirring at room temperature for 16 h, the mixture was used for next step directly.

### Step 5: anti-2-(1-(4-(trifluoromethyl)benzyl)-5-(4-(trifluoromethyl)phenyl) piperidin-3-yl)acetic acid

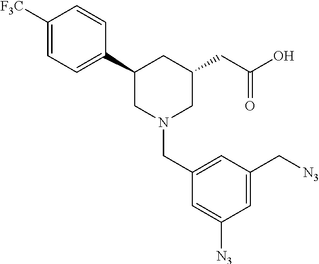 [SMILES: [N-]=[N+]=NCc1cc(CN2C[C@@H](CC(=O)O)C[C@H](c3ccc(C(F)(F)F)cc3)C2)cc(N=[N+]=[N-])c1]

To the DMF solution of step 4 were added MeOH (2 mL), H₂O (2 mL), and NaOH (8.00 mg, 0.2 mmol). The reaction was stirred at 35° C. for 2 h. The mixture was adjusted to pH=4˜5 and concentrated. The residue was purified via prep-HPLC to give the title compound. Prep-HPLC: (Method C). Yield: 28% (5.0 mg, white solid). ¹H NMR (400 MHz, DMSO-d₆) δ 7.64-7.56 (m, 4H), 7.14 (s, 1H), 7.04 (s, 2H), 6.99 (s, 1H), 4.46 (s, 2H), 3.61-3.57 (m, 2H), 3.12-3.08 (m 1H), 2.73-2.70 (m, 1H), 2.41-2.34 (m, 4H), 2.17 (s, 1H), 1.82-1.76 (m, 1H), 1.67-1.64 (m, 1H), 1.51-1.21 (m, 1H). LCMS: (Method E) 446.0 (M+H), Rt. 2.45 min, 99.06% (Max). HPLC: (Method C) Rt. 4.27 min, 98.77% (Max).

### 128

Chemical names and characterizations of all the compounds are shown in Table 3 below.

TABLE 3
Chemical Names

| Compound | | |
|---|---|---|
| No. | Chemical Name | MS Characterization |
| 100 | rac-2-((anti)-1-(4-(trifluoromethyl)benzyl)-5-(4- | LCMS: (Method A) 446.0 (M + H) |
| | (trifluoromethyl)phenyl)piperidin-3-yl)acetic acid | |
| 101 | 2-((3S,5S)-1-(4-(trifluoromethyl)benzyl)-5-(4- | LCMS: (Method D) 446.1 (M + H), Rt. |
| | (trifluoromethyl)phenyl)piperidin-3-yl)acetic acid | 2.24 min, 96.90% (Max) |
| 102 | rac-2-((3S,5R)-1-(4-(trifluoromethyl)benzyl)-5-(4- | LCMS: (Method C) 446.1 (M + H), Rt. |
| | (trifluoromethyl)phenyl)piperidin-3-yl)acetic acid | 1.61 min, 99.10% (Max) |
| 103 | 2-((anti)-5-(3-methoxyphenyl)-1-(4- | LCMS: (Method A) 408.0 (M + H), Rt. |
| | (trifluoromethyl)benzyl)piperidin-3-yl)acetic acid | 2.27 min, 96.96% (Max) |
| 104 | rac-2-((anti)-1-(4-(trifluoromethyl)benzyl)-5-(3- | LCMS: (Method A) 446.0 (M + H), Rt. |
| | (trifluoromethyl)phenyl)piperidin-3-yl)acetic acid | 2.45 min, 96.50% (Max) |
| 105 | 2-((anti)-5-(4-methoxyphenyl)-1-(4- | LCMS: (Method A) 408.0 (M + H), Rt. |
| | (trifluoromethyl)benzyl)piperidin-3-yl)acetic acid | 2.26 min, 98.85% (Max) |
| 106 | rac-2-((anti)-5-(3-methoxy-5- | LCMS: (Method A) 476.0 (M + H), Rt. |
| | (trifluoromethyl)phenyl)-1-(4- | 2.48 min, 99,60% (Max) |
| | (trifluoromethyl)benzyl)piperidin-3-yl)acetic acid | |
| 107 | 2-((anti)-5-(4-cyanophenyl)-1-(4- | LCMS: (Method A) 403.0 (M + H), Rt. |
| | (trifluoromethyl)benzyl)piperidin-3-yl)acetic acid | 2.23 min, 94.15. % (Max) |
| 108 | 2-((3S,5S)-1-(3-methoxy-5-(trifluoromethyl)benzyl)- | LCMS: (Method C) 476.0 (M + H), Rt. |
| | 5-(4-(trifluoromethyl)phenyl)piperidin-3-yl)acetic | 1.67 min, 99.27% (Max) |
| | acid | |
| 109 | 2-(3S,5S)-1-(4-methoxybenzy)-5-(4- | LCMS: (Method A) 408.2 (M + H), Rt. |
| | (trifluoromethyl)phenyl)piperidin-3-yl)acetic acid | 2.03 min, 99.84% (Max) |
| 110 | 2-((3S,5S)-1-(3-methoxybenzyl)-5-(4- | LCMS: (Method A) 408.0 (M + H), Rt. |
| | (trifluoromethyl)phenyl)piperidin-3-yl)acetic acid | 2.32 min, 99.17% (Max) |
| 111 | 2-((3S,5S)-1-(3-(trifluoromethyl)benzyl)-5-(4- | LCMS: (Method D) 446.1 (M + H), Rt |
| | (trifluoromethyl)phenyl)piperidin-3-yl)acetic acid | 2.27 min, 99.68% (Max) |
| 112 | 2-((3S,5S)-1-(4-ethynylbenzyl)-5-(4- | LCMS: (Method D) 402.1 (M + H), Rt. |
| | (trifluoromethyl)phenyl)piperidin-3-yl)acetic acid | 2.16 min, 99.36% (Max) |
| 113 | (R)-2-((3S,5S)-1-(4-(trifluoromethyl)benzyl)-5-(4- | LCMS: (Method A) 460.0 (M + H), Rt. |
| | (trifluoromethyl)phenyl)piperidin-3-yl)propanoic | 2.46 min, 98.45% (Max) |
| | acid & (S)-2-((3R,5S)-1-(4-(trifluoromethyl)benzyl)- | |
| | 5-(4-(trifluoromethyl)phenyl)piperidin-3- | |
| | yl)propanoic acid | |
| 114 | (R)-2-((3S,5S)-1-(4-(trifluoromethyl)benzyl)-5-(4- | LCMS: (Method A) 459.9 (M + H), Rt. |
| | (trifluoromethyl)phenyl)piperidin-3-yl)propanoic | 2.51 min, 96.48% (Max) |
| | acid | |
| 115 | 2-((3S,5S)-1-(4-cyanobenzyl)-5-(4- | LCMS: (Method A) 402.9 (M + H), Rt. |
| | (trifluoromethyl)phenyl)piperidin-3-yl)acetic acid | 2.26 min, 98.85% (Max) |
| 116 | 2-((3S,5S)-1-(4-chlorobenzyl)-5-(4- | MS: (Method H) 412.2 (M + H), Rt. 1.77 |
| | (trifluoromethyl)phenyl)piperidin-3-yl)acetic acid | min, 100.00% (Max) |
| 117 | 2-((3S,5S)-1-(4-fluorobenzyl)-5-(4- | LCMS: (Method H) 396.1 (M + H), Rt. |
| | (trifluoromethyl)phenyl)piperidin-3-yl)acetic acid | 1.73 min, 100.00% |
| 118 | 2-((3S,5S)-1-(3,4-difluorobenzyl)-5-(4- | LCMS: (Method H) 414.2 (M + H), Rt. |
| | (trifluoromethyl)phenyl)piperidin-3-yl)acetic acid | 1.83 min, 100.00% (Max) |
| 119 | 2-((3S,5S)-1-(2-fluorobenzyl)-5-(4- | LCMS: (Method H) 396.2 (M + H), Rt. |
| | (trifluoromethyl)phenyl)piperidin-3-yl)acetic acid | 1.75 min, 100.00% (Max) |
| 120 | 2-((3S,5S)-1-(4-(methylsulfonyl)benzyl)-5-(4- | LCMS: (Method H) 456.2 (M + H), Rt. |
| | (trifluoromethyl)phenyl)piperidin-3-yl)acetic acid | 1.46 min, 100.00% (Max) |
| 121 | Anti-2-(-5-(3,4-difluorophenyl)-1-(4- | LCMS: (Method H) 414.2 (M + H), Rt. |
| | (trifluoromethyl)benzyl)piperidin-3-yl)acetic acid | 1.66 min, 100.00% (Max) |
| 122 | Anti-2-(5-(4-fluorophenyl)-1-(4- | LCMS: (Method H) 396.2 (M + H), Rt. |
| | (trifluoromethyl)benzyl)piperidin-3-yl)acetic acid | 1.59 min, 100.00% (Max) |
| 123 | Anti-2-(-5-(4-chlorophenyl)-1-(4- | LCMS: (Method F) 412.2 (M + H), Rt. |
| | (trifluoromethyl)benzyl)piperidin-3-yl)acetic acid | 1.40 min, 97,0% (Max) |
| 124 | Anti-2-(-1-(3-cyanobenzyl)-5-(4- | LCMS: (Method E) 403.2 (M + H), Rt. |
| | (trifluoromethyl)phenyl)piperidin-3-yl)acetic acid | 1.44 min, 100.00% (Max) |
| 125 | Anti-2-(1-(4-(trifluoromethyl)benzyl)-5-(4- | LCMS: (Method F) 474.2 (M + H), Rt. |
| | (trifluoromethyl)phenyl)piperidin-3-yl)butanoic acid | 1.68 min, 100% (Max) |
| 126 | Anti-3-(1-(4-(trifluoromethyl)benzyl)-5-(4- | LCMS: (Method F) 480.2 (M + H), Rt. |
| | (trifluoromethyl)phenyl)piperidin-3-yl)propanoic | 1.44 min, 95,74% (Max) |
| | acid | |
| 127 | anti-2-(5-(3-cyanophenyl)1-(4- | LCMS: (Method H) 403.2 (M + H), Rt. |
| | (trifluoromethyl)benzyl)piperidin-3-yl)acetic acid | 0.90 min, 100.00% (Max) |
| 128 | Anti-2-(1-(2-fluoro-4-(trifluoromethyl)benzyl)-5-(4- | LCMS: (Method E) 464.2 (M + H), Rt. |
| | (trifluoromethyl)phenyl)piperidin-3-yl)acetic acid | 1.55 min, 100.00% (Max) |
| 129 | Anti-2-(5-(3-fluoro-4-(trifluoromethyl)phenyl)-1-(4- | LCMS: (Method H) 464.1 (M + H), Rt. |
| | (trifluoromethyl)benzyl)piperidin-3-yl)acetic acid | 0.99 min, 100.00% (Max) |
| 130 | Anti-2-(1-(3-fluoro-4-(trifluoromethyl)benzyl)-5-(4- | LCMS: (Method F) 464.2 (M + H), Rt. |
| | (trifluoromethyl)phenyl)piperidin-3-yl)acetic acid | 1.60 min, 97.17% (Max) |
| 131 | Anti-2-(-1-(2-chloro-4-(trifluoromethyl)benzyl)-5- | LCMS: (Method F) 480.2 (M + H), Rt. |
| | (4-(trifluoromethyl)phenyl)piperidin-3-yl)acetic acid | 1.69 min, 100% (Max) |
| 132 | anti-2-(1-(4-(trifluoromethyl)benzyl)-5-(4- | LCMS: (Method E) 446.0 (M + H), Rt. |
| | (trifluoromethyl)phenyl) piperidin-3-yl)acetic acid | 2.45 min, 99,06% (Max) |
| 133 | Anti-2-(1-(4-(trifluoromethyl)benzyl)-5-(4- | LCMS: (Method E) 474.3 (M + H), Rt. |
| | (trifluoromethyl)phenethyl)piperidin-3-yl)acetic acid | 1.97 min, 100% (Max) |
| 134 | anti-2-(1-(4-(azidomethyl)benzyl)-5-(4- | LCMS: (Method H) 406.3 (M + H), Rt. |
| | azidophenyl)piperidin-3-yl)acetic acid | 1.28 min, 100.00% (Max) |
| 135 | Anti-2-(1-(4-chlorobenzyl)-5-(4- | LCMS: (Method E) 378.2, 380.2(M + H), |
| | chlorophenyl)piperidin-3-yl)acetic acid | Rt. 1.25 min, 100.00% (Max) |
| 136 | Anti-2-(5-(4-ethynylphenyl)-1-(4-(3- | LCMS: (Method F) 442.2 (M + H), Rt. |
| | (trifluoromethyl)-3H-diazirin-3-yl)benzyl)piperidin- | 1.38 min, 100% (Max) |
| | 3-yl)acetic acid | |
| 137 | Anti-2-(1-(4-azidobenzyl)-5-(4- | LCMS: (Method F) 375.2 (M + H), Rt. |
| | ethynylphenyl)piperidin-3-yl)acetic acid | 1.44 min, 100% (Max) |
| 138 | Anti-2-(1-benzyl-5-(4- | LCMS: (Method F) 378.2 (M + H), Rt. |
| | (trifluoromethyl)phenyl)piperidin-3-yl)acetic acid | 1.31 min, 100% (Max) |
| 139 | 2-((3S,5S)-1-(4-chlorobenzyl)-5-(4- | LCMS: (Method F) 406.1 (M + H), Rt. |
| | chlorophenyl)piperidin-3-yl)acetic acid | 1.31 min, 97.21% (Max) |
| 140 | Anti-2-(1-(4-chloro-3-cyanobenzyl)-5-(4- | LCMS: (Method F) 437.0 (M + H), Rt. |
| | (trifluoromethyl)phenyl)piperidin-3-yl)acetic acid | 1.46 min, 100% (Max) |
| 141 | Anti-2-(1-(naphthalen-2-ylmethyl)-5-(4- | LCMS: (Method F) 428.2 (M + H), Rt. |
| | (trifluoromethyl)phenyl)piperidin-3-yl)acetic acid | 1.40 min, 100% (Max) |
| 142 | 2-((3S,5S)-1-(4-chlorobenzyl)-5-(4- | LCMS: (Method F) 412.0 (M + H), Rt. |
| | chlorophenyl)piperidin-3-yl)acetic acid | 1.43 min, 99.17% (Max) |
| 143 | Anti-2-(5-(3-chlorophenyl)-1-(4- | LCMS: (Method F) 412.0 (M + H), Rt. |
| | (trifluoromethyl)benzyl)piperidin-3-yl)acetic acid | 1.42 min, 99.07% (Max) |
| 144 | 2-((3S,5S)-5-(4-(azido)phenyl)-1-(4- | LCMS: (Method F) 375.2 (M + H), Rt. |
| | ethynylbenzyl)piperidin-3-yl)acetic acid | 1.35min, 91.42% (Max) |
| 145 | 2-((3S,5S)-1-(4-(azidomethyl)benzyl)-5-(4- | LCMS: (Method F) 406.2 (M + H), Rt. |
| | azidophenyl)piperidin-3-yl)acetic acid | 1.36 min, 100% (Max) |
| 146 | 2-((3S,5S)-5-(4-ethynylphenyl)-1-(4-(3- | LCMS: (Method F) 442.2 (M + H), Rt. |
| | (trifluoromethyl)-3H-diazirin-3-yl)benzyl)piperidin- | 1.38 min, 100% (Max) |
| | 3-yl)acetic acid | |

### Example 2. In Vitro Studies of the Compounds

HepG2 and HEK293T hTERT mRNA Assay

HepG2 cells (hepatocyte carcinoma (HCC) cells; with mutated TERT) or HEK293T cells (with wild type (wt) TERT) were seeded at 4,000 cells/well in 96-well plates. The following day, test compounds were added to cells for at concentrations of 25 uM, 12.5 uM, 6.25 uM, 3.125 uM, 1.563 uM, 0.781 uM, 0.391 uM, 0.195 uM, 0.098 uM or vehicle control (0.1% DMSO). Each condition was tested in three replicate wells. 48 hours post drug addition, the cells were harvested for RNA using the 96-Magmax RNA Isolation kit (Cat. No. AM1830). RNA was converted to cDNA using the Superscript III First strand cDNA synthesis kit (Cat. No. 18080051). RNA and cDNA concentration and quality were determined using the NanoQuant nucleic acid quantification method. 500 ng cDNA from each sample was used to detect TERT mRNA via qPCR. GUSB was also measured as an internal control and fold change to vehicle control was determined using the delta CT method. The data are shown in Table 4. In the table, “+++” means <0.5 uM; “++” means 0.5-5 uM; “+” means >5-25 uM; IA means inactive and ND means not determined.

TABLE 4
TERT inhibition in HepG2 cells and HEK293 cells

| | | Mutant | Cytostatic | wt | | wt Tert |
|---|---|---|---|---|---|---|
| | Chirally | TERT | IC50 uM | TERT | wt TERT | IC50 uM |
| Compound | pure | IC50 | HepG2 | IC50 | IC50 uM | HEK293 |
| ID | (y/n) | HepG2 | cytostatic | uM | HEK293 | cytostatic |
| 100 | N | “++” | “++”-“+” | >25 | IA | “++”-“+” |
| 101 | Y | “+++” | ND | | “++”-IA | ND |
| 102 | N | “+” | “+” | >25 | IA | “+” |
| 103 | N | IA | “+” | >25 | IA | “+” |
| 104 | N | | “+” | >25 | IA | “+” |
| 105 | N | “++” | “+” | >25 | IA | “+” |
| 106 | N | “++” | “++” | >25 | IA | “+” |
| 107 | N | “+” | “+” | >25 | IA | IA |
| 108 | Y | IA | IA | >25 | IA | ND |
| 109 | Y | IA | “++” | >25 | IA | “+” |
| 110 | Y | IA | ND | >25 | IA | ND |
| 111 | Y | “+++” | ND | >25 | IA | ND |
| 112 | Y | “+++” | “++” | >25 | IA | IA |
| 113 | Y | “++” | “++” | >25 | IA | “+” |
| 114 | Y | “++” | “++” | >25 | IA | “++” |
| 115 | Y | “++” | “++” | >25 | IA | “+” |
| 116 | N | ++ | | | | |
| 117 | Y | ++ | | | | |
| 118 | N | +++ | | | | |
| 119 | N | ++ | | | | |
| 120 | Y | ++ | | | | |
| 121 | N | ++ | | | | |
| 122 | N | ++ | | | | |
| 123 | N | +++ | | | | |
| 124 | N | +++ | | | | |
| 125 | Y | ++ | | | | |
| 126 | N | ++ | | | | |
| 127 | N | ++ | | | | |
| 128 | N | ++ | | | | |
| 129 | N | ++ | | | | |
| 130 | N | ++ | | | | |
| 131 | N | ++ | | | | |
| 132 | N | ++ | | | | |
| 133 | N | ++ | | | | |
| 134 | N | ++ | | | | |
| 135 | N | +++ | | | | |
| 136 | N | ++ | | | | |
| 137 | N | ++ | | | | |
| 138 | N | ++ | | | | |
| 139 | Y | ++ | | | | |
| 140 | N | ++ | | | | |
| 141 | N | ++ | | | | |
| 142 | Y | +++ | | | | |
| 143 | N | + | | | | |
| 144 | Y | ++ | | | | |
| 145 | N | ++ | | | | |
| 146 | Y | ++ | | | | |

### Example 3. In Vivo Studies of the Compounds in GBM Models GBM (Glioblastoma) Xenograft Models

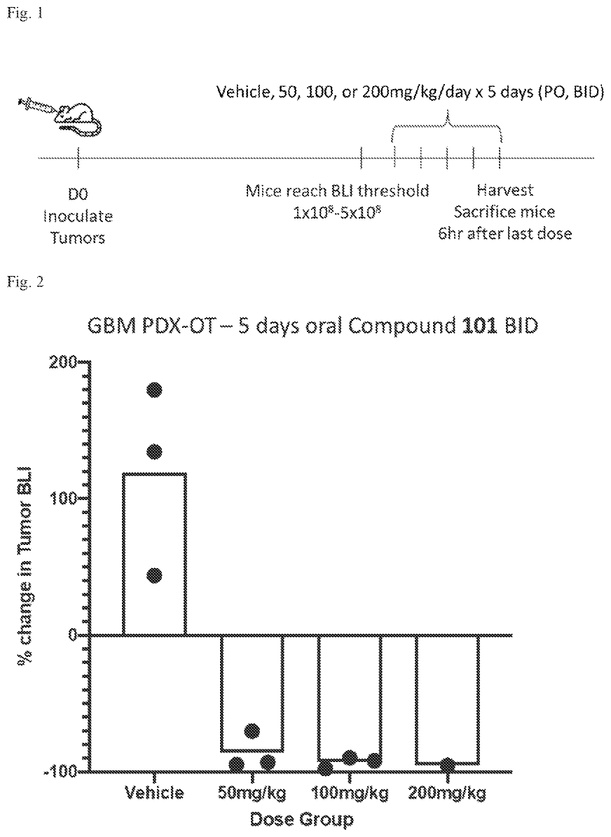

In this study, Compound 101 was dosed twice daily (BID) via oral gavage to mice with GBM39 orthotopic xenograft (GBM39 cells). Tumor size was measured via Bioluminescence imaging (BLI). BLI readings were taken on treatment day 1 and day 5. Tumors were also harvested for downstream RNA analysis. The study design is shown in FIG. 1.

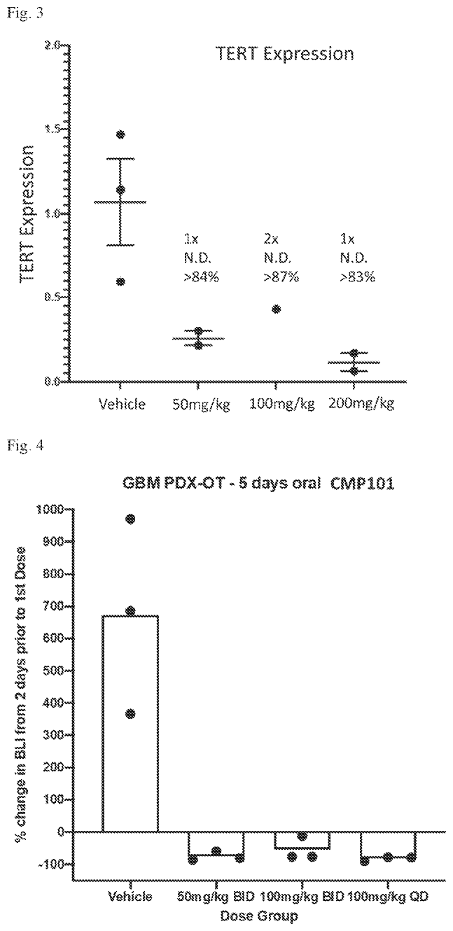

For each dose group (N=6), the following steps were performed. Briefly, athymic nu/nu mice were injected intracranially with patient-derived Glioblastoma cells. Animals began treatment once tumor BLI levels reach the range of 1×10⁸-5×10⁸. A dosing solution of Compound 101 (50 mg/kd, 100 mg/kd, or 200 mg/kg) or vehicle were administered by oral gavage (P.O., 50-100 uL volume) for 5 days. The dosing solutions were prepared similar to the process in Example 4. Dosing was twice daily on days 1-4. On day 5 the mice received a single dose, prior to harvest. Three hours after the last dose, mice were sacrificed and their brains were harvested and processed for analysis. As shown in FIG. 2, Compound 101 caused significant tumor reduction in 5 days. TERT expression was also found to be reduced in all three dose groups (FIG. 3).

In a second study with Compound 101 in the GBM mice model, the 200 mg/kg dosing group was changed to a 100 mg/kg once daily dosing (QD). The baseline BLI readings were taken two days prior to 1st treatment. Tumor BLI changes are shown in FIG. 4. Compound 101 again caused significant tumor reduction in 5 days. 100 mg/kg once daily dosing was sufficient to reduce tumor size.

### Example 4. In Vivo Studies of the Compounds in HepG2 Models

The pharmacodynamic (PD) profiling of Compound 101 was determined in Athymic nude mice bearing subcutaneous tumor xenograft model.

1. Preparation of Dosing Solution (25 mg/kg):

125 μL of Solutol HS-15 (CAS No. 70142-34-6) was added into the tube containing 3.125 mg of Compound 101 and vortexed well to dissolve. 62.5 μL of dimethyl sulfoxide (DMSO) was added and vortexed for 1 min. 1062.5 μL of water was added. The tube was vortexed for 1-2 min, heated to 50° C. and sonicated for 20 min. The data are given in Table 5.

TABLE 5
Preparation

| Weight of Compound 101 | 3.125 mg (for 5 animals) | |
|---|---|---|
| Purity of Compound 101 | 100% | |
| Concentration of Compound 101 | 2.5 | mg/mL |
| Vehicle | Solutol HS-15:DMSO:Water = | |
| | 10:5:85 (v/v/v) | |
| Volume of vehicle | 1.25 | mL |

2. Preparation of Dosing Solution (50 mg/kg):

125 μL of Solutol HS-15 was added into the tube containing 6.250 mg of Compound 101 and vortexed well to dissolve. 62.5 μL of DMSO was added and vortexed for 1 min. 1062.5 μL of water was added. The tube was vortexed for 1-2 min, heated to 50° C. and sonicated for 20 min. The data are given in Table 6.

TABLE 6
Preparation

| Weight of Compound 101 | 6.250 mg (for 5 animals) | |
|---|---|---|
| Purity of Compound 101 | 100% | |
| Concentration of Compound 101 | 5.0 | mg/mL |
| Vehicle | Solutol HS-15:DMSO:Water = | |
| | 10:5:85 (v/v/v) | |
| Volume of vehicle | 1.25 | mL |

HepG2 (Hepatocellular carcinoma cell line containing the C228T TERTp mutation) cells were implanted into the flanks of Athymic Nude-Foxn1nu mice. Once tumors reached 250-300 mm3 in size as measured by caliper, treatment of Compound 101 was initiated. Compound 101 was administered orally (P.O.) for 5 days BID at either 25 mg/kg, 50 mg/kg, or vehicle control (N=4 per group). Body weight and tumor size were measured on days 1, 3, and 5. Two hours after final dose administration, mice were sacrificed, and tumors extracted for RNA isolation and TERT mRNA expression analysis via RT-qPCR. Both TERT and GUSB were measured using SYBR green primers and relative TERT levels were analyzed using the delta delta CT method.

TERT expressions, tumor volume changes, and animal body weight changes are included in Tables 7-9 below. Oral administration of Compound 101 caused tumor regression and TERT mRNA reduction at both dose levels that were tested. The data are given in Tables 7, 8 and 9.

TABLE 7
TERT expression

| | Mean | Standard |
|---|---|---|
| TERT Expression (RT-qPCR) | Fold | Deviation |
| Vehicle | 1.01 | 0.18 |
| Compound 101 (25 mg/kg, p.o; BID for 5 days) | 0.21 | 0.12 |
| Compound 101 (50 mg/kg, p.o; BID for 5 days) | 0.19 | 0.08 |

TABLE 8
Mean Tumor Volume and SEM

| | Day 1 | Day 3 | Day 5 |
|---|---|---|---|
| Mean Tumor Volume (mm3) | | | |
| Group 1 - Vehicle Control | 251.15 | 269.68 | 290.12 |
| Group 2 - Compound 101 | 252.22 | 230.02 | 197.97 |
| (25 mg/kg, p.o; BID For 5 days) | | | |
| Group 3 - Compound 101 | 251.37 | 212.58 | 168.34 |
| (50 mg/kg, p.o; BID For 5 days) | | | |
| SEM | | | |
| Group 1 - Vehicle Control | 9.06 | 11.80 | 14.37 |
| Group 2 - Compound 101 | 8.52 | 10.03 | 9.08 |
| (25 mg/kg, p.o; BID For 5 days) | | | |
| Group 3 - Compound 101 | 7.56 | 9.83 | 10.75 |
| (50 mg/kg, p.o; BID For 5 days) | | | |

TABLE 9
Body Weight

| | DAY 1 | DAY 3 | DAY 5 |
|---|---|---|---|
| % Mean body weight change | | | |
| Group 1 - Vehicle Control | 0 | −1.56 | −5.15 |
| Group 2 - Compound 101 | 0 | −4.36 | −3.65 |
| (25 mg/kg, p.o; BID For 5 days) | | | |
| Group 3 - Compound 101 | 0 | −11.09 | −10.68 |
| (50 mg/kg, p.o; BID For 5 days) | | | |
| SEM | | | |
| Group 1 - Vehicle Control | 0 | 0.51 | 0.86 |
| Group 2 - Compound 101 | 0 | 3.14 | 2.54 |
| (25 mg/kg, p.o; BID For 5 days) | | | |
| Group 3 - Compound 101 | 0 | 2.36 | 3.13 |
| (50 mg/kg, p.o; BID For 5 days) | | | |

### Example 5. GBM Orthotopic Xenograft Survival Study

GBM39 cells, a human PDX model of GBM, were selected for this study. GBM39 cells were stably transduced (MOI=3) with Firefly Luciferase Lentifect Purified Lentiviral Particles (Genecopoiea, #LPP-FLUC-Lv105) and were verified to be expressing luciferase stably and at similar levels 48 h pre-xenograft. A total of 1-4×10E4 luciferase-expressing PDX cells in 4 uL volume were injected into the right frontal cortex of athymic nu/nu mice (6-7-wk.-old, female). Animals were randomized into treatment groups and dosing initiated once tumors reach an average BLI of 1×10⁸.

For this study, treatment began on day 7 post tumor cell implant and ceased on day 20 for a total of 14 days of dosing. Compound 101 was formulated with Solutol, DMSO and water and administered by oral gavage twice daily (BID). The dosage groups were vehicle control, 50 mg/kg BID, 25 mg/kg BID, and 10 mg/kg BID. The highest dosage group was changed to once daily dosing (qd) after the 3rd dosing day due to mice losing weight. Tumor size was monitored following injection of luciferin (150 mg/kg i.p.) 1-2 times weekly by bioluminescence imaging (Xenogen IVIS Spectrum Imaging System). Animals were weighed three times per week, and daily after initial weight loss is observed. The median survival of the animals is shown in the Table 10 below.

TABLE 10
Median Survival

| | | Compound 101 PoC, | Compound | Compound |
|---|---|---|---|---|
| | | 100 mg/kg/day, | 101 Poc, | 101 Poc, |
| | Vehicle | bid × 3 days. | 50 mg/ | 20 mg/ |
| | Only | then 50 mg/kg/day, | kg/day, | kg/day, |
| Group | (n = 10) | qd (n = 9) | bid (n = 10) | bid (n = 10) |
| Median | 20.5 | Undefined | 62.5 | 50 |
| Survival | | | | |

1. A compound having a structure of Formula (I): 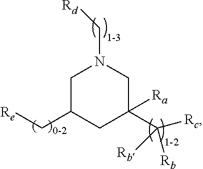
or a pharmaceutically acceptable salt thereof, wherein
Rₐ each individual R_{b} and each individual R_{b′} are independently H, halogen, or optionally substituted C₁-C₄ alkyl (e.g., methyl); wherein optionally Rₐ and R_{b} are joined to form a 5 or 6 membered ring;
R_{c} is carboxylic acid or its isostere;
R_{d} is an optionally substituted aryl or heteroaryl group; and
Rₑ is an optionally substituted aryl or heteroaryl group.

2. The compound of claim 1, wherein R_{d} or Rₑ is a phenyl group with at least one substituent in the ortho, para or meta position(s).

3. The compound of claim 2, wherein R_{d} or Rₑ is a phenyl group with 2 substituents, wherein the 2 substituents are in the (3, 5) or (3, 4) positions.

4. The compound of claim 2, wherein the substituent(s) for R_{d} or Rₑ is halogen, optionally substituted alkyl, optionally substituted alkenyl, optionally substituted alkynyl, optionally substituted alkoxyl, optionally substituted amine, optionally substituted amide, optionally substituted sulfone, optionally substituted heteroaryl, azide (N₃), nitrile (CN), Cl, F, or CF₃, and wherein the optional substituent includes hydroxyl, methoxy, ethoxy, dimethyl amino, diethyl amino, fluoro, chloro, bromo, CN, CONH₂, CON(CH₃)₂, SO₂NH₂, SO₂NHCH₃, or SO₂CH₃.

5. The compound of claim 1, wherein R_{b} is H, CH₃(Me) or F.

6. The compound of claim 1, wherein R_{b′} is H, Me or F.

7. The compound of claim 1, wherein R_{c} is —COOH.

8. The compound of claim 1, wherein R_{c} is hydroxamic acid, acylcyanamide, sulfonimide, phosphonate, sulfonate, sulfonamide, tetrazole, hydroxyisoxazole, or oxadiazolone.

9. The compound of claim 1, wherein the compound is Compound 100, 101, 102, 103, 104, 105, 106, 107, 108, 109, 110, 111, 112, 113, 114, 115, 116, 117, 118, 119, 120,121, 122, 123, 124, 125, 126, 127, 128, 129, 130, 131, 132, 133, 134, 135, 136, 137, 138, 139, 140, 141, 142, 143, 144, 145, 146, 147, 148 or 149.

10. A compound having a structure of Formula (II): 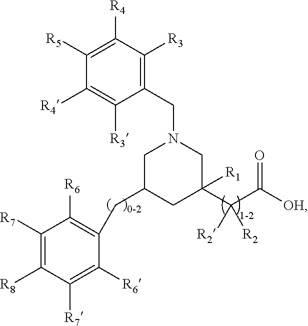
or a pharmaceutically acceptable salt thereof, wherein
R₁ each individual R₂ and each individual R_{2′} are independently H, halogen, or optionally substituted C₁-C₄ alkyl; wherein optionally R₁ and R₂ are joined to form a 5 or 6 membered ring;
R₃, R_{3′}, R₄, R_{4′} or R₅ is independently hydrogen, halogen, optionally substituted alkyl, optionally substituted alkenyl, optionally substituted alkynyl, optionally substituted alkoxyl, optionally substituted amine, optionally substituted amide, optionally substituted sulfone, optionally substituted heteroaryl, azide (N₃), nitrile (CN), or CF₃; wherein optionally R₄ and R₅ together with the carbon atoms they are attached to form an optionally substituted aromatic ring, wherein at least 3 of R₃, R_{3′}, R₄, R_{4′} and R₅ are H; and
R₆, R_{6′}, R₇, R_{7′} or R₈ is independently hydrogen, halogen, optionally substituted alkyl, optionally substituted alkenyl, optionally substituted alkynyl, optionally substituted alkoxyl, optionally substituted amine, optionally substituted amide, optionally substituted sulfone, optionally substituted heteroaryl, azide (N₃), nitrile (CN), or CF₃, wherein at least 3 of R₆, R_{6′}, R₇, R_{7′} and R₈ are H.

11. The compound of claim 10, wherein R₁ is H, —CH₃ or —CH₂OH.

12. The compound of claim 10, wherein R₂ is H, —CH₃, —CH₂CH₃ or —F.

13. The compound of claim 10, wherein both R₁ and R₂ are H.

14. The compound of claim 10, wherein R_{2′} is H, Me or F.

15. The compound of claim 10, wherein 3 of R₃, R_{3′}, R₄, R_{4′} or R₅ are H; the other two are independently —CF₃, —OMe, Cl, —CN, F, SO₂Me, N₃, CH₂N₃,

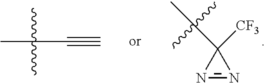

16. The compound of claim 10, wherein 4 of R₃, R_{3′}, R₄, R_{4′} or R₅ are H; the other one is —CF₃, —OMe, Cl, —CN, F, SO₂Me, N₃, CH₂N₃,

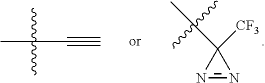

17. The compound of claim 10, wherein R₃ and R_{3′} are H; R₄, R_{4′} or R₅ is independently H, —CF₃, —OMe, —CN, F, SO₂Me, N₃, CH₂N₃, 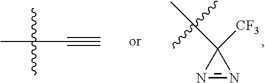
wherein at least one of R₄, R_{4′} or R₅ is H.

18. The compound of claim 10, wherein 3 of R₆, R_{6′}, R₇, R_{7′} or R₈ are H; the other two are independently —CF₃, —OMe, —CN, F, Cl, N₃ or

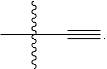 [SMILES: *C#C]

19. The compound of claim 10, wherein 4 of R₆, R_{6′}, R₇, R_{7′} or R₈ are H; the other one is —CF₃, —OMe, —CN, F, Cl, N₃ or

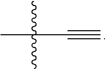 [SMILES: *C#C]

20. The compound of claim 10, wherein R₆ and R_{6′} are H; R₇, R_{7′} or R₈ is independently H, —CF₃, —OMe, —CN, F, Cl, N₃ or 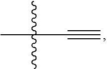 [SMILES: *C#C]
wherein at least one of R₇, R_{7′} or R₈ is H.

21. The compound of claim 10, wherein the compound is Compound 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148 or 149.

22. A compound having a structure of Formula (III): 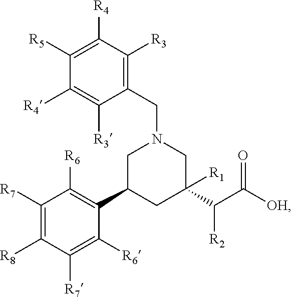
or a pharmaceutically acceptable salt thereof, wherein
R₁ and R₂ are independently H or optionally substituted C₁-C₄ alkyl;
wherein optionally R₁ and R₂ are joined to form a 5 or 6 membered ring;
R₃, R_{3′}, R₄, R_{4′} or R₅ is independently hydrogen, halogen, optionally substituted alkyl, optionally substituted alkenyl, optionally substituted alkynyl, optionally substituted alkoxyl, optionally substituted amine, optionally substituted amide, optionally substituted sulfone, optionally substituted heteroaryl, azide (N), nitrile (CN), or CF₃, wherein at least 3 of R₃, R_{3′}, R₄, R_{4′} and R₅ are H; and
R₆, R_{6′}, R₇, R_{7′} or R₈ is independently hydrogen, halogen, optionally substituted alkyl, optionally substituted alkenyl, optionally substituted alkynyl, optionally substituted alkoxyl, optionally substituted amine, optionally substituted amide, optionally substituted sulfone, optionally substituted heteroaryl, azide (N₃), nitrile (CN), or CF₃, wherein at least 3 of R₆, R_{6′}, R₇, R_{7′} and R₈ are H.

23. The compound of claim 22, wherein R₁ is H, —CH₃ or —CH₂OH.

24. The compound of claim 22, wherein R& is H, —CH₃ or —CH₂CH₃.

25. The compound of claim 22, wherein both R₁ and R₂ are H.

26. The compound of claim 22, wherein 3 of R₃, R_{3′}, R₄, R_{4′} or R₅ are H; the other two are independently —CF₃, —OMe, —CN, —Cl, —F, —SO₂Me, —N₃, —CH2N3,

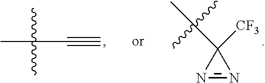

27. The compound of claim 22, wherein 4 of R₃, R_{3′}, R₄, R_{4′} or R₅ are H; the other one is —CF₃, —OMe, —CN, —Cl, —F, —SO₂Me, —N₃, —CH₂N₃,

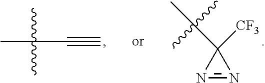

28. The compound of claim 22, wherein R₃ and R_{3′} are H; R₄, R_{4′} or R₅ is independently H, —CF₃, —OMe, —CN, —Cl, —F, —SO₂Me, —N₃, —CH2N3, 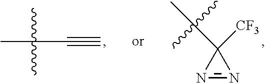
wherein at least one of R₄, R_{4′} or R₅ is H.

29. The compound of claim 22, wherein 3 of R₆, R_{6′}, R₇, R_{7′} or R₈ are H; the other two are independently —CF₃, —OMe, —CN, —Cl, —F, —SO₂Me, —N₃, —CH₂N₃,

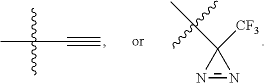

30. The compound of claim 22, wherein 4 of R₆, R_{6′}, R₇, R_{7′} or R₈ are H; the other one is —CF₃, —OMe, —CN, —Cl, —F, —SO₂Me, —N₃, —CH₂N₃,

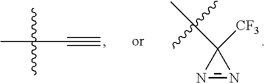

31. The compound of claim 22, wherein R₆ and R_{6′} are H; R₇, R_{7′} or R₈ is independently H, —CF₃, —OMe, —CN, —Cl, —F, —SO₂Me, —N₃, —CH₂N₃, 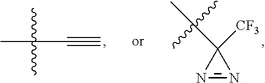
wherein at least one of R₇, R_{7′} or R₈ is H.

32. The compound of claim 22, wherein the compound is Compound 100, 101, 102, 103, 104, 105, 106, 107, 108, 109, 110, 111, 112, 113, 114, 115, 116, 117, 118, 119, 120, 121, 122, 123, 124, 125, 127, 128, 129, 130, 131, 132, 134, 135, 136, 137, 138, 139, 140, 142, 143, 144, 145, 146 or 148.

33. A compound selected from the group consisting of Compound 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, and 149, or a pharmaceutically acceptable salt thereof.

34. A pharmaceutical composition comprising the compound of claim 1, and a pharmaceutically acceptable carrier.

35. The pharmaceutical composition of claim 34, wherein the carrier comprises water.

36. The pharmaceutical composition of claim 35, wherein the carrier further comprises solutol.

37. The pharmaceutical composition of claim 35, wherein the carrier further comprises dimethyl sulfoxide (DMSO).

38. A method of inhibiting the expression of the telomerase reverse transcriptase (TERT) gene or reducing the amount of TERT mRNA or TERT protein in a cell, comprising administering an effective amount of the compound of claim 1.

39. The method of claim 38, wherein the TERT gene in the cell has a mutant promoter.

40. The method of claim 38, wherein the TERT gene in the cell does not have a mutant promoter.

41. The method of claim 38, wherein the cell is a cancer cell.

42. The method of claim 41, wherein the cancer cell is a glioblastoma cell, a cancer cell of anus, bladder, bile duct, bone, brain, breast, cervix, colon/rectum, endometrium, esophagus, eye, gallbladder, head and neck, liver, kidney, larynx, lung, mediastinum, mouth, ovaries, pancreas, penis, prostate, skin, small intestine, stomach, spinal marrow, tailbone, testicles, thyroid or uterus.

43. The method of claim 38, wherein the cell is a central nervous system (CNS) tumor cell.

44. The method of claim 38, wherein the cell is a hepatocellular carcinoma cell.

45. A method of treating cancer, reducing tumor volume, reducing tumor growth, or increasing survival of a subject in need thereof, comprising administering to the subject an effective amount of the compound of claim 1.

46. The method of claim 45, wherein the cancer is glioblastoma, a cancer in anus, bladder, bile duct, bone, brain, breast, cervix, colon/rectum, endometrium, esophagus, eye, gallbladder, head and neck, liver, kidney, larynx, lung, mediastinum, mouth, ovaries, pancreas, penis, prostate, skin, small intestine, stomach, spinal marrow, tailbone, testicles, thyroid or uterus.

47. The method of claim 45, wherein the cancer is a CNS cancer.

48. The method of claim 45, wherein the cancer is hepatocellular carcinoma.
